(19) United States
(12) Patent Application Publication
(10) Pub. No.: US 2014/0329858 A1
(43) Pub. Date: Nov. 6, 2014

(54) Cyclic Urea Derivatives As Androgen Receptor Antagonists

(71) Applicants: Mark Gary Bock, Boston, MA (US); Dinesh Chikkanna, Bangalore (IN); Marc Gerspacher, Hägendorf (CH); Vinayak Khairnar, Bangalore (IN); Bharat Lagu, Lexington, MA (US); Chetan Pandit, Bangalore (IN)

(72) Inventors: Mark Gary Bock, Boston, MA (US); Dinesh Chikkanna, Bangalore (IN); Marc Gerspacher, Hägendorf (CH); Vinayak Khairnar, Bangalore (IN); Bharat Lagu, Lexington, MA (US); Chetan Pandit, Bangalore (IN)

(73) Assignee: NOVARTIS AG, Basel (CH)

(21) Appl. No.: 14/361,728

(22) PCT Filed: Dec. 3, 2012
(86) PCT No.: PCT/IB2012/056927

§ 371 Date: May 30, 2014

(30) Foreign Application Priority Data
Dec. 5, 2011 (IN) 3525/DEL/2011

(51) Int. Cl. C07D 235/02; C07D 401/04; C07D 409/04; C07D 491/052; C07D 401/12; C07D 401/06; C07D 401/14; C07D 405/04; C07D 405/12

(52) U.S. Cl. CPC C07D 235/02; C07D 405/04; C07D 401/04; C07D 409/04; C07D 405/12; C07D 401/12; C07D 401/06; C07D 401/14; C07D 491/052
USPC 514/322; 548/302.7; 514/387; 546/273.7; 514/338; 546/199; 548/303.1

(57) ABSTRACT

The present invention is directed to compounds of formula (I) wherein R¹, R², R³, and A are defined herein. The present invention also provides for pharmaceutical compositions comprising a compound of formula (I) as well as to the use of such compounds as androgen receptor antagonists for the treatment of diseases and conditions mediated by the androgen receptor, such as prostate cancer.

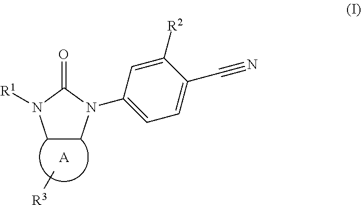

### FIELD OF THE INVENTION

The present invention relates to certain cyclic urea derivatives, compositions containing them, and the use of such compounds as androgen receptor antagonists for the treatment of diseases and conditions mediated by the androgen receptor, such as prostate cancer.

### BACKGROUND OF THE INVENTION

Androgen receptor (AR), a steroid hormone receptor, is a ligand-dependent transcription factor that mediates androgen action in cells. AR is found in the cytoplasm bound to heat shock proteins which stabilize the receptor and allow androgen binding. Once androgen binds to AR, the receptor dimerizes and moves to the nucleus where it induces transcription of target genes involved in cell cycle regulation and proliferation. AR is found in a variety of tissues throughout the human body, including muscles, skin, scalp, and prostate.

Androgen receptor is the primary therapeutic target in prostate cancer. The first course of treatment in primary prostate cancer is androgen abalation therapy (AAT). AAT consists of one or more combinations of GnRH agonists (to suppress pituitary signaling), aromatase inhibitors (to decrease androgen production), and competitive AR antagonists, such as hydroxy-flutamide or bicalutamide (to block AR directly). Initially AAT is effective in controlling the disease, but over time tumor cells evolve mechanisms for continued growth under conditions of androgen depletion and the cancer becomes what is known as recurrent or hormone-refractory prostate cancer (HRPC). However, the growth of most HRPC is dependent on AR-mediated signaling. Such AR signaling includes up-regulation of AR protein expression levels, acquisition of mutations within AR that increase its activity in response to alternative hormones (including antagonists), or up-regulation of co-activator proteins that augment AR activity. Thus, it is likely that new approaches to block AR activity, including the discovery of better competitive AR antagonists, could significantly extend or increase the effectiveness of AAT. This suggests that novel AR antagonists could have considerable utility in the treatment of both primary and recurrent prostate cancer.

Androgen receptor also plays an important role in many other male hormone related diseases including benign prostate hypertrophy, male hair loss, muscle loss and hirsutism. Thus, androgen receptor antagonists may be useful for the treatment of conditions and diseases including but not limited to male contraception, a variety of male hormone related conditions such as hypersexuality and sexual deviation; benign prostate hyperplasia, acne vugaris, androgenetic alopecia, and hirsutism. Androgen receptor antagonists could also be used in preventing the symptoms associated with reduced testosterone such as hot flashes after castration and for purposefully presenting or counteracting masculinisation in the case of transsexual women undergoing sex reassignment therapy.

Thus, there is a significant medical need for better androgen receptor antagonists.

### SUMMARY OF THE INVENTION

The present invention is directed to compounds of formula (I). The present invention also provides for pharmaceutical compositions comprising a compound of formula (I) as well as to the use of such compounds as androgen receptor antagonists for the treatment of diseases and conditions mediated by the androgen receptor such as, prostate cancer.

### DETAILED DESCRIPTION OF THE INVENTION

The present invention is directed to a compound of formula (I):

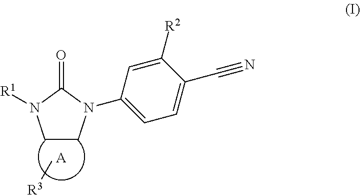

wherein:
R¹ is C₁₋₃alkyl or optionally substituted phenyl, optionally substituted benzyl, optionally substituted 2,3-dihydrobenzofuranyl, optionally substituted 5 or 6 membered heteroaryl, or optionally substituted 5 or 6 membered heteroaryl-CH₂—, wherein each ring is optionally substituted with one to three substituents each independently selected from the group consisting of: halo, cyano, hydroxy, C₁₋₃alkyl optionally substituted with one hydroxy group, C₁₋₃alkoxy, C₁₋₃haloalkyl, cyclopropyl, imidazolyl, pyrazolyl, triazolyl, tetrazolyl, morpholinyl, tetrahydrofuranyl, piperidinyl, piperazinyl, oxetanyl, C(O)R^{a}, NR^{a}R^{a}, COOR^{a}, C(O)NR^{a}R^{b}, C(O)NR^{a}OR^{c}, C(S)NR^{a}R^{b}, NR^{a}C(O)R^{a}, NHSO²R^{a}, and SO²NR^{a}R^{a};
R² is halo, C₁₋₃alkyl or C₁₋₃haloalkyl;
ring A is cyclohexane, cycloheptane, cyclohexene, cycloheptene, or a 6 or 7 membered saturated monocyclic heterocyclic ring having one heteroatom selected from the group consisting of O and S;
R³ is H, hydroxy, oxo, C₁₋₃alkyl, C₁₋₃alkoxy, optionally substituted phenyl, or optionally substituted 5 or 6 membered heteroaryl, wherein each ring is optionally substituted with one to three substituents each independently selected from the group consisting of: halo, cyano, hydroxy, C₁₋₃alkyl optionally substituted with one hydroxy group, C₁₋₃alkoxy, C₁₋₃haloalkyl, cyclopropyl, imidazolyl, pyrazolyl, triazolyl, tetrazolyl, morpholinyl, tetrahydrofuranyl, piperidinyl, piperazinyl, oxetanyl, C(O)R^{a}, NR^{a}R^{a}, COOR^{a}, C(O)NR^{a}R^{b}, C(O)NR^{a}OR^{b}, C(S)NR^{a}R^{b}, NR^{a}C(O)R^{a}, NHSO₂R^{a}, and SO₂NR^{a}R^{a};
R^{a} is H or C₁₋₃alkyl;
R^{b} is H, tetrahydrofuranyl, piperidinyl, piperazinyl, or oxetanyl or R^{b} is C₁₋₃alkyl optionally substituted with one or two substituents each independently selected from the group consisting of: hydroxy and C₁₋₃alkoxy;
R^{c} is C₁₋₄alkyl optionally substituted with one substituent selected from the group consisting of: hydroxy, N(CH₃)₂, N(CH₂CH₃)₂, tetrahydrofuranyl, C₁₋₄alkoxy, and C₃₋₅cycloalkyl, or R^{c} is tetrahydrofuranyl or piperidinyl, said piperidinyl being optionally substituted with one C₁₋₃alkyl group.

“Alkyl” refers to a monovalent saturated hydrocarbon chain having the specified number of carbon atoms. For example, C₁₋₃alkyl refers to an alkyl group having from 1 to 3 carbon atoms. Alkyl groups may be optionally substituted with one or more substituents as defined in formula (I). Alkyl groups may be straight or branched. Representative alkyl groups have one or two branches. Alkyl includes methyl, ethyl, and propyl (n-propyl and iso-propyl), butyl (n-butyl, i-butyl, sec-butyl, and t-butyl).

“Alkoxy” refers to an alkyl moiety attached through an oxygen bridge (i.e. a —O—C₁₋₃alkyl wherein C₁₋₃alkyl is defined herein). Examples of alkoxy groups include methoxy, ethoxy, and propoxy.

“Cycloalkyl” refers to a saturated hydrocarbon ring system having the specified number of carbon atoms. For example, C₃₋₅ cycloalkyl refers to a cycloalkyl group having from 3 to 5 carbon atoms. Cycloalkyl groups may be optionally substituted with one or more substituents as defined in formula (I). Examples of cycloalkyl groups include, but are not limited to, cyclopropyl, cyclobutyl, cyclopentyl, cyclohexyl, and cycloheptyl.

“Halo” refers to the halogen radicals fluoro, chloro, bromo, and iodo.

“Haloalkyl” refers to an alkyl group wherein at least one hydrogen atom attached to a carbon atom within the alkyl group is replaced with a halo. The number of halo substituents includes but are not limited to 1, 2, 3, 4, 5, or 6 substituents. Haloalkyl includes monofluoromethyl, difluoroethyl, and trifluoromethyl.

“Heteroaryl” refers to an aromatic ring containing from 1 to 4, suitably 1 or 2 heteroatoms as member atoms in the ring. Heteroaryl groups containing more than one heteroatom may contain different heteroatoms. Heteroaryl groups may be optionally substituted with one or more substituents as defined in formula (I). Five or six membered heteroaryl rings are monocyclic. Examples of 5 and 6 membered heteroaryl groups include, but are not limited to, pyrrolyl, pyrazolyl, imidazolyl, oxazolyl, isoxazolyl, oxadiazolyl, furanyl, thienyl, triazolyl, pyridinyl, pyrimidinyl, pyridazinyl, pyrazinyl, triazinyl, tetrazinyl, and tetrazolyl.

“Heteroatom” refers to a nitrogen, sulfur, or oxygen atom.

“Heterocyclic” refers to a saturated or unsaturated ring system containing from 1 to 4 heteroatoms. Heterocyclic ring systems are not aromatic. Heterocyclic groups containing more than one heteroatom may contain different heteroatoms. Heterocyclic includes ring systems wherein a sulfur atom is oxidized to form SO or SO₂. Heterocyclic groups may be optionally substituted with one or more substituents as defined in formula (I). Heterocyclic groups are monocyclic ring systems, spirocycle, or bridged bicyclic ring systems. Monocyclic heterocyclic rings have from 4 to 7 member atoms. Bicyclic heterocyclic rings have 6 or 7 member atoms. Heterocyclic includes, among others, pyrrolidinyl, tetrahydrofuranyl, dihydrofuranyl, pyranyl, tetrahydropyranyl, dihydropyranyl, tetraydrothienyl, pyrazolidinyl, oxazolidinyl, thiazolidinyl, piperidinyl, piperizinyl, morpholinyl, thiamorpholinyl, and azepinyl.

“Optionally substituted” indicates that a group such as alkyl, phenyl, heteroaryl, and heterocyclic may be unsubstituted, or the group may be substituted with one or more substituents as defined.

“Substituted” in reference to a group such as alkyl, phenyl, heteroaryl, and heterocyclic, indicates that one or more hydrogen atoms attached to an atom within the group is replaced with a substituent selected from the group of defined substituents. It should be understood that the term “substituted” includes the implicit provision that such substitution be in accordance with permitted valence of the substituted atom and the substituent, and that the substitution results in a stable compound (i.e. one that does not spontaneously undergo transformation, for example, by hydrolysis, rearrangement, cyclization, or elimination and that is sufficiently robust to survive isolation from a reaction mixture). When it is stated that a group may contain one or more substituents, one or more (as appropriate) atoms within the group may be substituted. In addition, a single atom within the group may be substituted with more than one substituent as long as such substitution is accordance with the permitted valence of the atom. Suitable substituents are defined for each substituted or optionally substituted group.

The skilled artisan will appreciate that salts, including pharmaceutically acceptable salts, of the compounds according to formula (I) may be prepared. These salts may be prepared in situ during the final isolation and purification of the compound, or by separately reacting the purified compound in its free acid or free base form with a suitable base or acid, respectively.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Solvates, including pharmaceutically acceptable solvates, of the compounds of formula (I) may also be prepared. “Solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

As used herein, the term “pharmaceutically acceptable” means a compound which is suitable for pharmaceutical use. Salts and solvates (e.g. hydrates and hydrates of salts) of compounds of the invention which are suitable for use in medicine are those where in the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

The compounds of formula (I), including salts and solvates thereof, may exist in crystalline forms, non-crystalline forms, or mixtures thereof. The compound or salt or solvate thereof may also exhibit polymorphism, i.e. the capacity of occurring in different crystalline forms. These different crystalline forms are typically known as “polymorphs”. Polymorphs have the same chemical composition but differ in packing, geometrical arrangement, and other descriptive properties of crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, all of which may be used for identification. One of ordinary skill in the art will appreciate that different polymorphs may be produced, for example, by changing or adjusting the conditions used in crystallizing/recrystallizing a compound of formula (I).

The invention also includes various isomers of the compounds of formula (I). “Isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of formula (I) may have one or more asymmetric carbon atom and may occur as racemates, racemic mixtures and as individual enantiomers or diastereomers. All such isomeric forms are included within the present invention, including mixtures thereof. If the compound contains a double bond, the substituent may be in the E or Z configuration. If the compound contains a disubstituted cycloalkyl, the cycloalkyl substituent may have a cis- or trans-configuration. All tautomeric forms are also intended to be included.

Any asymmetric atom (e.g., carbon or the like) of a compound of formula (I) can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of formula (I) can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

The invention includes unlabeled forms as well as isotopically labeled forms of compounds of formula (I). Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Furthermore, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### REPRESENTATIVE EMBODIMENTS

Various embodiments of the invention are described herein. It will be recognized that features specified in each embodiment may be combined with other specified features to provide for further embodiments.

One embodiment of the present invention is a compound according to formula (Ia) wherein the stereocenters marked by a * are in the trans configuration

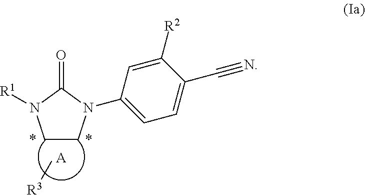

Another embodiment of the present invention is a compound according to formula (Ib):

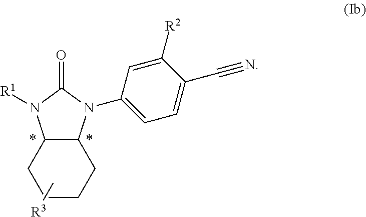

Another embodiment is a compound according to formula (Ic):

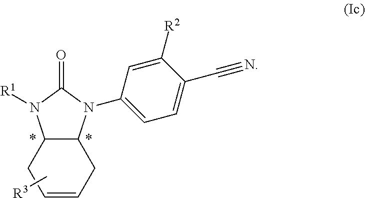

Another embodiment is a compound according to formula (Id):

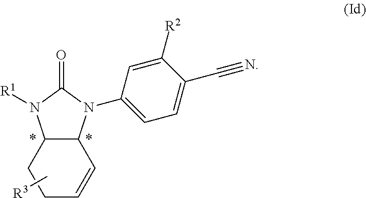

Another embodiment is a compound according to formula (Ie):

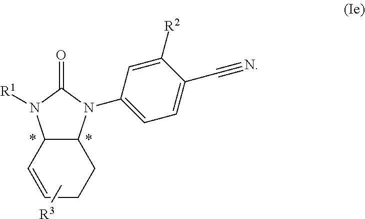

Another embodiment is a compound of formula (If):

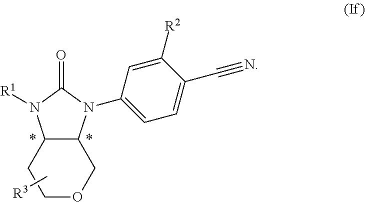

Another embodiment is a compound of formula (Ig):

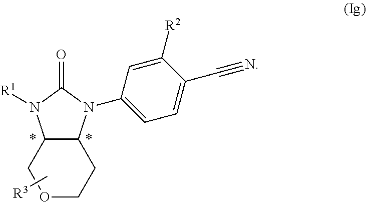

Another embodiment is a compound of formula (Ih):

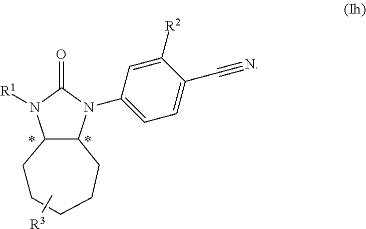

In another embodiment of the present invention, R¹ is optionally substituted phenyl, optionally substituted 2,3-dihydrobenzofuranyl, or optionally substituted 5-6 membered heteroaryl. Suitably, R¹ is optionally substituted phenyl, optionally substituted 2,3-dihydrobenzofuranyl, optionally substituted furanyl, optionally substituted imidazolyl, optionally substituted thienyl, or optionally substituted pyridinyl. More suitably R¹ is optionally substituted phenyl, optionally substituted furan-3-yl, optionally substituted imidazol-1-yl, optionally substituted thien-3-yl, optionally substituted pyridin-2-yl, optionally substituted pyridin-3-yl, or optionally substituted pyridinyl-4-yl. More suitably R¹ is phenyl, furan-3-yl, imidazol-1-yl, thien-3-yl, pyridin-2-yl, pyridin-3-yl, or pyridinyl-4-yl each of which is optionally substituted with one to three, suitably one or two, substituents each independently selected from the group consisting of: fluoro, chloro, cyano, methyl, trifluoromethyl, cyclopropyl, imidazolyl, pyrazolyl, C(O)NHOR^{c}, NH₂, NHCH₃, COOH, C(O)CH₃, CH₂OH, COOCH₂CH₃, C(O)NR^{a}R^{b}, SO₂NH₂, NHC(O)CH₃, N(CH₃)C(O)CH₃, and NHSO₂CH₃, C(S)NHCH₃.

In another embodiment R¹ is optionally substituted benzyl, optionally substituted pyridinyl-CH₂, or optionally substituted imidazolyl-CH₂—. In another embodiment R¹ is unsubstituted benzyl, unsubstituted pyridinyl-CH₂, or unsubstituted imidazolyl-CH₂—.

In another embodiment R¹ is:

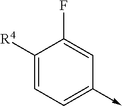

wherein the arrow indicates the point of attachment to formula (I) and R⁴ is halo, cyano, hydroxy, C₁₋₃alkyl optionally substituted with one hydroxy group, C₁₋₃alkoxy, C₁₋₃haloalkyl, cyclopropyl, imidazolyl, C(O)R^{a}, NR^{a}R^{a}, COOR^{a}, C(O)NR^{a}R^{b}, C(O)NR^{a}OR^{c}, C(S)NR^{a}R^{b}, NR^{a}C(O)R^{a}, NHSO₂R^{a}, and SO₂NR^{a}R^{a}. Suitably R⁴ is C(O)H, CH2OH, C(O)NHOR^{c}, NH₂, COOH, NHCH₃, C(O)NH₂, C(O)NHCH₃, C(O)NHCH₂CH₂OH, NHC(O)CH₃, N(CH₃)C(O)CH₃, NCOOCH2CH3, or NHSO₂CH₃C(S)NHCH₃. More suitably R⁴ is C(O)NH₂, C(O)NHCH₃, or C(O)NHCH₂CH₂OH.

In another embodiment R¹ is C₁₋₃alkyl. Suitably R¹ is methyl.

In another embodiment R² is C₁₋₃haloalkyl. Suitably R² is CF₃.

In another embodiment R³ is oxo, hydroxy, methoxy, or unsubstituted phenyl.

In another embodiment R³ is H.

In another embodiment R^{a} is H, methyl, or ethyl.

In another embodiment R^{b} is H, methyl, CH₂CH₂OH, tetrahydrofuranyl, or CH(CH₂CH₂OH)₂.

In another embodiment R^{c} is methyl optionally substituted with one cyclopropyl, cyclobutyl, methoxy, or tetrahydrofuranyl group; ethyl substituted with one methoxy group; propyl substituted with one butoxy, hydroxy, dimethylamino, or diethylamino group; butyl; or 1-methyl-piperidinyl.

Specific compounds of the present invention include:
Trans-4-{3-[4-cyano-3-(trifluoromethyl)phenyl]-2-oxo-octahydro-1H-1,3-benzodiazol-1-yl}2-fluoro-N-Methylbenzamide (±);
Trans-4-{3-[4-cyano-3-(trifluoromethyl)phenyl]-2-oxo-octahydro-1H-1,3-benzodiazol-1-yl}-2-fluoro-N-Methylbenzamide (+);
Trans-4-{3-[4-cyano-3-(trifluoromethyl)phenyl]-2-oxo-octahydro-1H-1,3-benzodiazol-1-yl}-2-fluoro-N-Methylbenzamide (−);
Trans-4,4′-(2-oxohexahydro-1H-benzo[d]imidazole-1,3(2H)-diyl)bis(2-(trifluoromethyl)benzonitrile) (±);
Trans-4,4′-(2-oxohexahydro-1H-benzo[d]imidazole-1,3(2H)-diyl)bis(2-(trifluoromethyl)benzonitrile) (−);
Trans-4,4′-(2-oxohexahydro-1H-benzo[d]imidazole-1,3(2H)-diyl)bis(2-(trifluoromethyl)benzonitrile) (+);
Trans-4-(3-(furan-3-yl)-2-oxoocta-hydro-1H-benzo[d]imidazol-1-yl)-2-trifluoromethyl)benzonitrile (±);
Trans-4-(2-oxo-3-(pyridin-4-yl)octahydro-1H-benzo[d]imidazol-1-yl)-2-(trifluoromethyl)benzonitrile (±);
Trans-4-(2-oxo-3-phenyloctahydro-1H-benzo[d]imidazol-1-yl)-2-(trifluoromethyl)benzonitrile (±);
Trans-4-(2-oxo-3-phenyloctahydro-1H-benzo[d]imidazol-1-yl)-2-(trifluoromethyl)benzonitrile (+);
Trans-4-(2-oxo-3-phenyloctahydro-1H-benzo[d]imidazol-1-yl)-2-(trifluoromethyl)benzonitrile (−);
Trans-ethyl-4-(3-(4-cyano-3-trifluoromethyl)phenyl)-2-oxooctahydro-1H-benzo[d]imidazol-1-yl)-2-fluorobenzoate (±);
Trans-ethyl-4-{3-[4-cyano-3-(trifluoromethyl)phenyl]-2-oxo-octahydro-1H-1,3-benzodiazol-1-yl}-2-methylbenzoate (±);
Trans-ethyl-5-(3-(4-cyano-3-(trifluoromethyl)phenyl)-2-oxooctahydro-1H-benzo[d]imidazol-1-yl)thiophene-2-carboxylate (±);
Trans-6-(3-(4-cyano-3-(trifluoromethyl)phenyl)-2-oxo octahydro-1H-benzo[d]imidazol-1-yl) pyridine-2-sulfonamide (±);
Trans-4-(3-(2-fluoropyridin-4-yl)-2-oxooctahydro-1H-benzo[d]imidazol-1-yl)-2-(trifluoromethyl)benzonitrile (±);
Trans-4-(3-(2-fluoropyridin-4-yl)-2-oxooctahydro-1H-benzo[d]imidazol-1-yl)-2-(trifluoromethyl)benzonitrile (+);
Trans-4-(3-(2-fluoropyridin-4-yl)-2-oxooctahydro-1H-benzo[d]imidazol-1-yl)-2-(trifluoromethyl)benzonitrile (−);
Trans-4-(2-oxo-3-(2-(trifluoromethyl)pyridin-4-yl)octahydro-1H-benzo[d]imidazol-1-yl)-2-(trifluoromethyl)benzonitrile (±);
Trans-N-(4-(3-(4-cyanophenyl)-2-oxooctahydro-1H-benzo[d]imidazol-1-yl)-2-fluorophenyl)acetamide (±);
Trans-N-(4-(3-(4-cyano-3-(trifluoromethyl)phenyl)-2-oxooctahydro-1H-benzo[d]imidazol-1-yl)-2-fluorophenyl)-N-methylacetamide (±);
Trans-4-(3-(3-fluoro-4-(methylamino)phenyl)-2-oxooctahydro-1H-benzo[d]im-idazol-1-yl)-2-(trifluoromethyl)benzonitrile (±);
Trans-(4-(-3-(4-amino-3-fluorophenyl)-2-oxooctahydro-1H-benzo[d]imidazol-1-yl)-2-(trifluoromethyl)benzonitrile (±);
Trans-N-(4-(3-(4-cyano-3-(trifluoromethyl)phenyl)-2-oxooctahydro-1H-benzo[d]imidazol-1-yl)-2-fluorophenyl)methanesulfonamide (±);
Trans-4-(3-(4-cyano-3-(trifluoromethyl)phenyl)-2-oxooctahydro-1H-benzo[d]im-idazol-1-yl)-2-fluorobenzoic acid (±);
Trans-4-(3-(4-cyano-3-(trifluoromethyl)phenyl)-2-oxooctahydro-1H-benzo[d]im-idazol-1-yl)-2-fluoro-N-methoxybenzamide (±);
Trans-4-(3-(4-cyano-3-(trifluoromethyl)phenyl)-2-oxooctahydro-1H-benzo[d]im-idazol-1-yl)-N-(cyclopropylmethoxy)-2-fluorobenzamide (±);
Trans-4-(3-(4-cyano-3-(trifluoromethyl)phenyl)-2-oxooctahydro-1H-benzo[d]im-idazol-1-yl)-2-fluoro-N-(2-methoxyethoxy)benzamide (±);
Trans-4-(3-(4-cyano-3-(trifluoromethyl)phenyl)-2-oxooctahydro-1H-benzo[d]im-idazol-1-yl)-N-(cyclobutylmethoxy)-2-fluorobenzamide (±);
Trans-4-(3-(4-cyano-3-(trifluoromethyl)phenyl)-2-oxooctahydro-1H-benzo[d]im-idazol-1-yl)-2-fluoro-N-isobutoxybenzamide (±);
Trans-N-((1-(tert-butoxy)propan-2-yl)oxy)-4-(3-(4-cyano-3-(trifluoromethyl)phenyl)-2-oxooctahydro-1H-benzo[d]imidazol-1-yl)-2-fluorobenzamide (±);
Trans-4-(3-(4-cyano-3-(trifluoromethyl)phenyl)-2-oxooctahydro-1H-benzo[d]im-idazol-1-yl)-2-fluoro-N-((1-hydroxypropan-2-yl)oxy)benzamide (±);
Trans 4-(3-(4-cyano-3-(trifluoromethyl)phenyl)-2-oxooctahydro-1H-benzo[d]im-idazol-1-yl)-N-(3-(dimethylamino)propoxy)-2-fluorobenzamide (±);
Trans-4-(3-(4-cyano-3-(trifluoromethyl)phenyl)-2-oxooctahydro-1H-benzo[d]im-idazol-1-yl)-N-(2-(diethylamino)ethoxy)-2-fluorobenzamide (±);
Trans 4-(3-(4-cyano-3-(trifluoromethyl)phenyl)-2-oxooctahydro-1H-benzo[d]im-idazol-1-yl)-2-fluoro-N-((tetrahydrofuran-2-yl)methoxy)benzamide (±);
Trans 4-(3-(4-cyano-3-(trifluoromethyl)phenyl)-2-oxooctahydro-1H-benzo[d]im-idazol-1-yl)-2-fluoro-N-((1-methylpiperidin-4-yl)oxy)benzamide (±);
4-((3aS,7aS)-3-(4-Cyano-3-(trifluoromethyl)phenyl)-2-oxooctahydro-1H-benzo[d]imidazol-1-yl)-2-fluoro-N-(2-methoxyethoxy)benzamide;
Trans-4-(3-(4-cyano-3-(trifluoromethyl)phenyl)-2-oxooctahydro-1H-benzo[d]im-idazol-1-yl)-2-fluorobenzamide (±);
4-((3aS,7aS)-3-(4-cyano-3-(trifluoromethyl)phenyl)-2-oxooctahydro-1H-benzo[d]imidazol-1-yl)-2-fluorobenzamide;
Trans 4-(3-(4-cyano-3-(trifluoromethyl)phenyl)-2-oxooctahydro-1H-benzo[d]im-idazol-1-yl)-2-fluoro-N-(2-hydroxyethyl)benzamide (±);
4-((3aS,7aS)-3-(4-cyano-3-(trifluoromethyl)phenyl)-2-oxooctahydro-1H-benzo[d]imidazol-1-yl)-2-fluoro-N-(2-hydroxyethyl)benzamide;
4-((3aR,7aR)-3-(4-cyano-3-(trifluoromethyl)phenyl)-2-oxooctahydro-1H-benzo[d]imidazol-1-yl)-2-fluoro-N—((R)-tetrahydrofuran-3-yl)benzamide and 4-((3aS,7aS)-3-(4-cyano-3-(trifluoromethyl)phenyl)-2-oxooctahydro-1H-benzo[d]imidazol-1-yl)-2-fluoro-N—((R)-tetrahydrofuran-3-yl)benzamide (mixture);
4-((3aS,7aS)-3-(4-cyano-3-(trifluoromethyl)phenyl)-2-oxooctahydro-1H-benzo[d]imidazol-1-yl)-2-fluoro-N—((S)-tetrahydrofuran-3-yl)benzamide and 4-((3aS,7aS)-3-(4-cyano-3-(trifluoromethyl)phenyl)-2-oxooctahydro-1H-benzo[d]imidazol-1-yl)-2-fluoro-N—((R)-tetrahydrofuran-3-yl)benzamide (mixture);
4-((3aS,7aS)-3-(4-cyano-3-(trifluoromethyl)phenyl)-2-oxooctahydro-1H-benzo[d]imidazol-1-yl)-2-fluoro-N—((R)-tetrahydrofuran-3-yl)benzamide;
4-((3aS,7aS)-3-(4-cyano-3-(trifluoromethyl)phenyl)-2-oxooctahydro-1H-benzo[d]imidazol-1-yl)-N-(1,3-dihydroxypropan-2-yl)-2-fluorobenzamide;
Trans-4-{3-[4-cyano-3-(trifluoromethyl)phenyl]-2-oxo-octahydro-1H-1,3-benzodiazol-1-yl}-2-methylbenzoic acid (±);
Trans-4-(3-(4-cyano-3-(trifluoromethyl)phenyl)-2-oxooctahydro-1H-benzo[d]im-idazol-1-yl)-N,2-dimethylbenzamide (±);
Trans-5-(3-(4-cyano-3-(trifluoromethyl)phenyl)-2-oxooctahydro-1H-benzo[d]imidazol-1-yl)-N-methylthiophene-2-carboxamide (±);
4-((3aS,7aS)-3-(4-cyano-3-(trifluoromethyl)phenyl)-2-oxooctahydro-1H-benzo[d]imidazol-1-yl)-2-fluoro-N-methylbenzothioamide;
Trans-2-chloro-4-(3-(4-cyano-3-(trifluoromethyl)phenyl)-2-oxooctahydro-1H-benzo[d]imidazol-1-yl)-3-methylbenzonitrile (±);
Trans-4-(3-(3,5-dichlorophenyl)-2-oxooctahydro-1H-benzo[d]imidazol-1-yl)-2-(trifluoro-methyl)benzonitrile (±);
4-((3aS,7aS)-3-(3,5-dichlorophenyl)-2-oxooctahydro-1H-benzo[d]imidazol-1-yl)-2-(trifluoromethyl)benzonitrile;
4-((3aS,7aS)-3-(4-acetyl-3-fluorophenyl)-2-oxooctahydro-1H-benzo[d]imidazol-1-yl)-2-(trifluoromethyl)benzonitrile;
4-((3aS,7aS)-3-(3-fluoro-4-(hydroxymethyl)phenyl)-2-oxooctahydro-1H-benzo[d]imidazol-1-yl)-2-(trifluoromethyl)benzonitrile;
Trans-4-(3-((6-chloropyridin-3-yl)methyl)-2-oxooctahydro-1H-benzo[d]imidazol-1-yl)-2-(trifluoromethyl)benzonitrile (±);
Trans-5-((-(4-cyano-3-(trifluoromethyl)phenyl)-2-oxooctahydro-1H-benzo[d]im-idazol-1-yl)methyl)picolinonitrile (±);
Trans-4-(3-((6-(1H-imidazol-1-yl)pyridin-3-yl)methyl)-2-oxooctahydro-1H-benzo[d]imidazol-1-yl)-2-(trifluoromethyl)benzonitrile (±);
Trans-4-(3-((6-(1H-pyrazol-1-yl)pyridin-3-yl)methyl)-2-oxooctahydro-1H-benzo[d]imidazol-1-yl)-2-(trifluoromethyl)benzonitrile (±);
Trans-4-(3-((1H-pyrazol-3-yl)methyl)-2-oxooctahydro-1H-benzo[d]imidazol-1-yl)-2-(trifluoromethyl)benzonitrile (±);
trans-4-(3-(3-methyl-1H-pyrazol-4-yl)-2-oxooctahydro-1H-benzo[d]imidazol-1-yl)-2-(trifluoromethyl)benzonitrile (±);
Trans-5-(3-(4-cyano-3-(trifluoromethyl)phenyl)-2-oxooctahydro-1H-benzo[d]imidazol-1-yl)-N-methylpicolinamide (±);
5-((3aS,7aS)-3-(4-cyano-3-(trifluoromethyl)phenyl)-2-oxooctahydro-1H-benzo[d]imidazol-1-yl)-N-methylpicolinamide;
5-((3aS,7aS)-3-(4-cyano-3-(trifluoromethyl)phenyl)-2-oxooctahydro-1H-benzo[d]imidazol-1-yl)picolinamide;
4-((3aS,7aS)-3-(4-cyano-3-(trifluoromethyl)phenyl)-2-oxooctahydro-1H-benzo[d]imidazol-1-yl)-N-methylpicolinamide;
4-((3aS,7aS)-3-(4-cyano-3-(trifluoromethyl)phenyl)-2-oxooctahydro-1H-benzo[d]imidazol-1-yl)picolinamide;
4-((3aS,7aS)-3-(2,3-dihydrobenzofuran-5-yl)-2-oxooctahydro-1H-benzo[d]imidazol-1-yl)-2-(trifluoromethyl)benzonitrile;
Trans-4-(3-(2-fluoropyridin-3-yl)-2-oxooctahydro-1H-benzo[d]imidazol-1-yl)-2-(trifluoromethyl)benzonitrile (±);
Trans-4-(3-(4-chlorophenyl)-2-oxooctahydro-1H-benzo[d]imidazol-1-yl)-2-(trifluoromethyl)benzonitrile (±);
4-((3aS,7aS)-3-(4-chlorophenyl)-2-oxooctahydro-1H-benzo[d]imidazol-1-yl)-2-(trifluoromethyl)benzonitrile;
4-((3aS,7aS)-3-(4-cyclopropylpyridin-3-yl)-2-oxooctahydro-1H-benzo[d]imidazol-1-yl)-2-(trifluoromethyl)benzonitrile;
4-((3aS,7aS)-3-(4-methylpyridin-3-yl)-2-oxooctahydro-1H-benzo[d]imidazol-1-yl)-2-(trifluoromethyl)benzonitrile;
Ethyl 4-((3aS,7aS)-3-(4-cyano-3-(trifluoromethyl)phenyl)-2-oxooctahydro-1H-benzo[d]imidazol-1-yl)benzoate;
4-((3aS,7aS)-3-(4-cyano-3-(trifluoromethyl)phenyl)-2-oxooctahydro-1H-benzo[d]imidazol-1-yl)benzoic acid;
4-((3aS,7aS)-3-(4-cyano-3-(trifluoromethyl)phenyl)-2-oxooctahydro-1H-benzo[d]imidazol-1-yl)benzamide;
Trans-4-(3-(4-cyano-3-(trifluoromethyl)phenyl)-2-oxooctahydro-1H-benzo[d]imidazol-1-yl)-2-fluorobenzenesulfonamide (±);
Ethyl 4-((3aS,7aS)-3-(4-cyano-3-(trifluoromethyl)phenyl)-2-oxooctahydro-1H-benzo[d]imidazol-1-yl)-2-methylbenzoate
4-((3aS,7aS)-3-(4-cyano-3-(trifluoromethyl)phenyl)-2-oxooctahydro-1H-benzo[d]imidazol-1-yl)-2-methylbenzoic acid;
4-((3aS,7aS)-3-(4-cyano-3-(trifluoromethyl)phenyl)-2-oxooctahydro-1H-benzo[d]imidazol-1-yl)-2-methylbenzamide;
Trans-4-{3-[4-Cyano-3-(trifluoromethyl)phenyl]-2-oxo-octahydropyrano[3,4-d]imidazolidin-1-yl}-2-fluoro-N-methylbenzamide (±);
Trans-4-{3-[4-Cyano-3-(trifluoromethyl)phenyl]-2-oxo-octahydropyrano[3,4-d]imidazolidin-1-yl}-2-fluoro-N-methylbenzamide (+);
Trans-4-{3-[4-Cyano-3-(trifluoromethyl)phenyl]-2-oxo-octahydropyrano[3,4-d]imidazolidin-1-yl}-2-fluoro-N-methylbenzamide (−);
Trans-4-(1-(2-methylpyridin-4-yl)-2-oxohexahydropyrano[3,4-d]imidazol-3(2H)-yl)-2-(trifluoromethyl)benzonitrile (±);
Trans-4-(1-(4-Cyano-3-(trifluoromethyl)phenyl)-2-oxohexahydropyrano[3,4-d]imidazol-3(2H)-yl)-2-fluoro-N-methylbenzamide (±);
Trans-ethyl 4-(3-(4-cyano-3-(trifluoromethyl)phenyl)-2-oxohexahydropyrano[3,4-d]imidazol-1(6H)-yl)-2-fluorobenzoate (±);
Trans-4-(3-(4-cyano-3-(trifluoromethyl)phenyl)-2-oxohexahydropyrano[3,4-d]imidazol-1(6H)-yl)-2-fluorobenzoic acid (±);
Trans-4-(3-(4-cyano-3-(trifluoromethyl)phenyl)-2-oxohexahydropyrano[3,4-d]imidazol-1(6H)-yl)-2-fluorobenzamide (±);
Trans-ethyl-4-(-3-(4-cyano-3-(trifluoromethyl)phenyl)-2-oxohexahydropyrano[3,4-d]imidazol-1(6H)-yl)-2-methylbenzoate (±);
Trans-4-(3-(4-cyano-3-(trifluoromethyl)phenyl)-2-oxohexahydropyrano[3,4-d]imidazol-1(6H)-yl)-2-methylbenzoic acid (±);
Trans-4-(3-(4-cyano-3-(trifluoromethyl)phenyl)-2-oxohexahydropyrano[3,4-d]imidazol-1(6H)-yl)-2-methylbenzamide (±);
Trans-4-(3-(4-cyano-3-(trifluoromethyl)phenyl)-2-oxooctahydrocyclohepta[d]imidazol-1(2H)-yl)-2-fluoro-N-methylbenzamide (±);
Trans-4-(3-(4-cyano-3-(trifluoromethyl)phenyl)-2-oxooctahydrocyclohepta[d]imidazol-1(2H)-yl)-2-fluoro-N-methylbenzamide (−);
Trans-4-(3-(4-cyano-3-(trifluoromethyl)phenyl)-2-oxooctahydrocyclohepta[d]imidazol-1(2H)-yl)-2-fluoro-N-methylbenzamide (+);
Trans-4-(3-(4-cyano-3-(trifluoromethyl)phenyl)-2,6-dioxooctahydro-1H-benzo[d]imidazol-1-yl)-2-fluoro-N-methylbenzamide (±);
Trans-4-(3-(4-cyano-3-(trifluoromethyl)phenyl)-6-hydroxy-2-oxooctahydro-1H-benzo[d]imidazol-1-yl)-2-fluoro-N-methylbenzamide (±);
Trans-4-(3-(4-cyano-3-(trifluoromethyl)phenyl)-6-hydroxy-2-oxooctahydro-1H-benzo[d]imidazol-1-yl)-2-fluoro-N-methylbenzamide (+);
Trans-4-(3-(4-cyano-3-(trifluoromethyl)phenyl)-6-hydroxy-2-oxooctahydro-1H-benzo[d]imidazol-1-yl)-2-fluoro-N-methylbenzamide (−);
Trans-4-(3-(4-cyano-3-(trifluoromethyl)phenyl)-2-oxo-2,3,3a,4,7,7a-hexahydro-1H-benzo[d]imidazol-1-yl)-2-fluoro-N-methylbenzamide (±);
trans-4-(3-(4-cyano-3-(trifluoromethyl)phenyl)-2-oxo-2,3,3a,4,5,7a-hexahydro-1H-benzo[d]imidazol-1-yl)-2-fluoro-N-methylbenzamide (±);
trans-4-3-(4-cyano-3-(trifluoromethyl)phenyl)-2-oxo-2,3,3a,4,7,7a-hexahydro-1H-benzo[d]imidazol-1-yl)-2-fluoro-N-methylbenzamide (+);
trans-4-3-(4-cyano-3-(trifluoromethyl)phenyl)-2-oxo-2,3,3a,4,7,7a-hexahydro-1H-benzo[d]imidazol-1-yl)-2-fluoro-N-methylbenzamide (−);
Trans-4-(3-(4-cyano-3-(trifluoromethyl)phenyl)-5-methoxy-2-oxooctahydro-1H-benzo[d]imidazol-1-yl)-2-fluoro-N-methylbenzamide (±);
trans-4-(3-(4-cyano-3-(trifluoromethyl)phenyl)-2-oxo-2,3,3a,6,7,7a-hexahydro-1H-benzo[d]imidazol-1-yl)-2-fluoro-N-methylbenzamide (±);
Cis- and trans-4-(3-methyl-2,5-dioxooctahydro-1H-benzo[d]imidazol-1-yl)-2-(trifluoromethyl)benzonitrile;
Trans-4-(1-(4-cyano-3-(trifluoromethyl)phenyl)-3-methyl-2-oxo-2,3,3a,6,7,7a-hexahydro-1H-benzo[d]imidazol-5-yl)-2-fluoro-N-methylbenzamide (±);
Cis-4-(1-(4-cyano-3-(trifluoromethyl)phenyl)-3-methyl-2-oxo-2,3,3a,6,7,7a-hexahydro-1H-benzo[d]imidazol-5-yl)-2-fluoro-N-methylbenzamide (±);
Trans-4-(3-methyl-2-oxo-5-phenyloctahydro-1H-benzo[d]imidazol-1-yl)-2-(trifluoromethyl)benzonitrile; and
Trans-4-3-methyl-2-oxo-5-phenyl-2,3,3a,6,7,7a-hexahydro-1H-benzo[d]imidazol-1-yl)-2-(trifluoromethyl)benzonitrile (±).

Preferred compounds of the invention include:
Trans-4-{3-[4-cyano-3-(trifluoromethyl)phenyl]-2-oxo-octahydro-1H-1,3-benzodiazol-1-yl}-2-fluoro-N-Methylbenzamide (±);
4-((3aS,7aS)-3-(4-cyano-3-(trifluoromethyl)phenyl)-2-oxooctahydro-1H-benzo[d]imidazol-1-yl)-2-fluorobenzamide; and
4-((3aS,7aS)-3-(4-cyano-3-(trifluoromethyl)phenyl)-2-oxooctahydro-1H-benzo[d]imidazol-1-yl)-2-fluoro-N-(2-hydroxyethyl)benzamide.

Another preferred compound is: Trans-4-(3-(4-cyano-3-(trifluoromethyl)phenyl)-2-oxooctahydro-1H-benzo[d]im-idazol-1-yl)-N,2-dimethylbenzamide (±).

Another preferred compound is: Trans-4-(3-(4-cyano-3-(trifluoromethyl)phenyl)-2-oxohexahydropyrano[3,4-d]imidazol-1(6H)-yl)-2-fluorobenzamide (±).

### General Synthetic Procedures

The compounds of the present invention may be made by a variety of methods, including standard chemistry. Illustrative general synthetic methods are set out below and specific compounds of the invention as prepared are given in the Examples.

The compounds of formula (I) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of formula (I).

Those skilled in the art will recognize if a stereocenter exists in the compounds of formula (I). Accordingly, the present invention includes all possible stereoisomers and includes not only racemic compounds but the individual enantiomers and diastereomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, “Stereochemistry of Organic Compounds” by E. L. Eliel, S. H. Wilen, and L. N. Mander (Wiley-interscience, 1994).

The compounds described herein may be made from commercially available starting materials or synthesized using known organic, inorganic, and/or enzymatic processes.

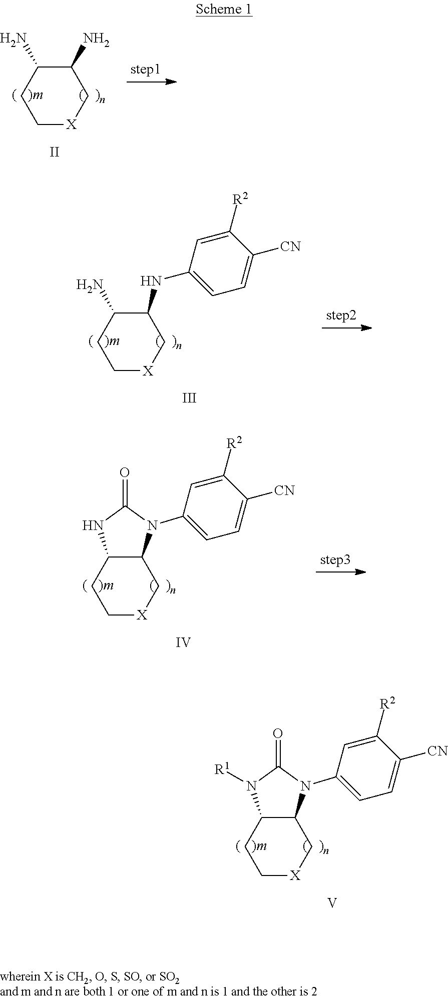

Vicinal diamines of formula II can be prepared by the methods known in the literature. For example, direct reaction of olefins with azide anion gives rise to vicinal diazides under transition metal oxidation with Mn(III), Fe(III), or Pb(IV). Alternatively, vicinal diazides can be prepared from epoxides via hydroxyazide intermediates or from vicinal dihalides via bimolecular nucleophilic substitution (referred to as “Sn2”) reactions. The vicinal diazides can be reduced to amines of formula II.

There are several indirect methods of preparing vicinal diamines from olefins. One such method converts olefins to iodocarbamates in a rather cumbersome manner involving iodoisocyanation and methanolysis of the isocyanate. Treatment of the iodocarbamate with hydroxide results in the formation of an aziridine which can be opened with ammonia to give vicinal diamines stereospecifically. Another method involves cycloaddition of chlorosulfonyl isocyanate to the olefin followed by a Curtius rearrangement and hydrolysis of the resulting cyclic urea. A third method involves the preparation of vicinal diamines from olefins and cyanamide/N-bromosuccinimide. A fourth method using olefins involves preparation from dienes via a Diels-Alder adduct of sulfur dioxide bis-imides.

Step1: Compounds of formula II can be converted in to compounds for formula III by reacting with the appropriate alkyl or aryl halides preferably chloro bromo alkyl or aryl derivatives using conditions well known in the literature for e.g., the Buchwald-Hartwig C—N coupling conditions or NaH/DMF, and the like. Preferred conditions are those known as the ‘Buclhvald-Hartwig” reaction, e.g., in the presence of (a) a catalyst, such as copper iodide, (b) a base, such as potassium phosphate or cesium carbonate: and (c) a ligand such as trans-1,2-diaminocyclohexane, 2-diamino cyclohexane in the presence of suitable solvents (e.g., 1,4-dioxane) at temperatures ranging from about room temperature to the refluxing temperature of the solvent. When a protection group is used, then the protecting group is removed using the conditions appropriate to the particular protecting group used to produce compounds of formula III.

Step2: Treatment of compounds for formula III with a reagent like CDI, phosgene, or triphosgene in the presence of a base like TEA produces the cyclized and N-substituted ureas of formula IV

Step3: Compounds of formula V can be synthesized from compounds of formula IV by following the methods described in Step 1.

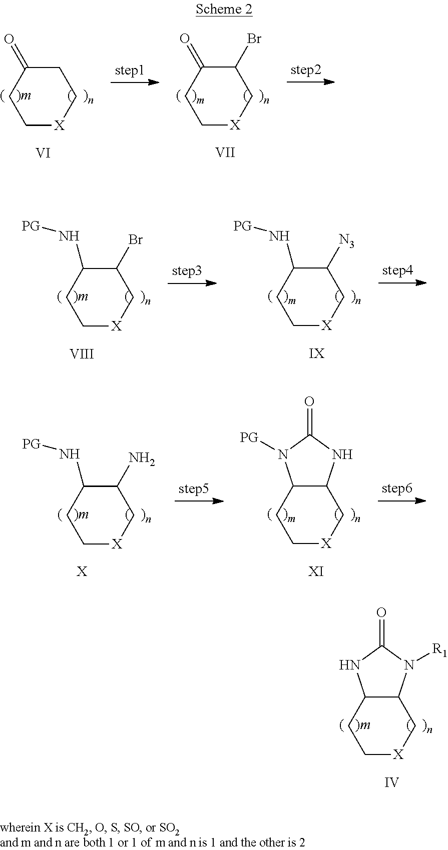

Another method of preparing N-substituted ureas of formula IV include reacting the compounds of formula XI with the appropriate alkyl or aryl halides as described in the steal of scheme-1 followed by standard deprotection of the protecting group. Compounds of formula XI could be obtained from diamines of formula X using the reagents like CDI, phosgene, or triphosgene in the presence of a base like TEA. Another method of preparing vicinal diamines of formula X apart from the methods mentioned in scheme 1 is reductive amination of an α-halo ketone of formula VII, followed by halo displacement by azide and subsequent reduction of azide to amine functionality. α-halo ketone of formula VII which in turn can be prepared from corresponding the ketones via standard halogenations methods.

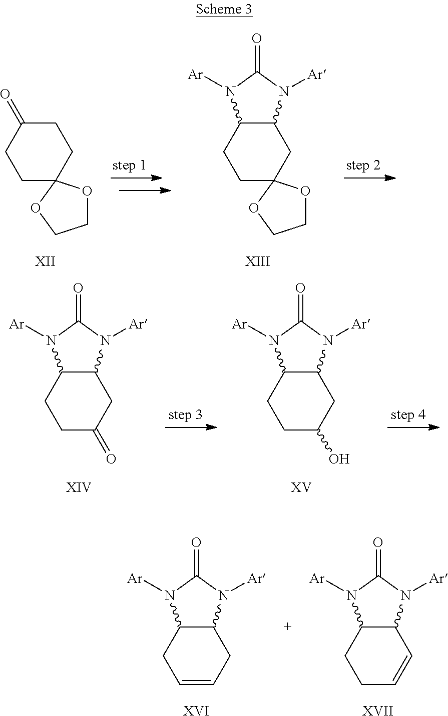

Ketal protected Compounds of formula XIII could be synthesized from corresponding starting materials of formula XII as described previously in schemes 1 and 2. Ketal deprotection of compounds of formula XIII could be achieved using the standard conditions known in the literature for example, Conc. HCl to give the ketones of formula XIV. Ketones of formula XIV could be converted to alcohols of formula XV using reducing agents such as NaBH₄. Olefins of formula XVI and XVII could be obtained from alcohols of formula XV using standard elimination reactions such as treatment with concentrated acids or via the reactive intermediates such as mesylates and tosylates. Optically pure compounds can be synthesized from the corresponding enantiopure starting materials or the racemic products can be resolved by standard techniques such as Chiral HPLC, crystallization, chromatography, and enzymatic separations.

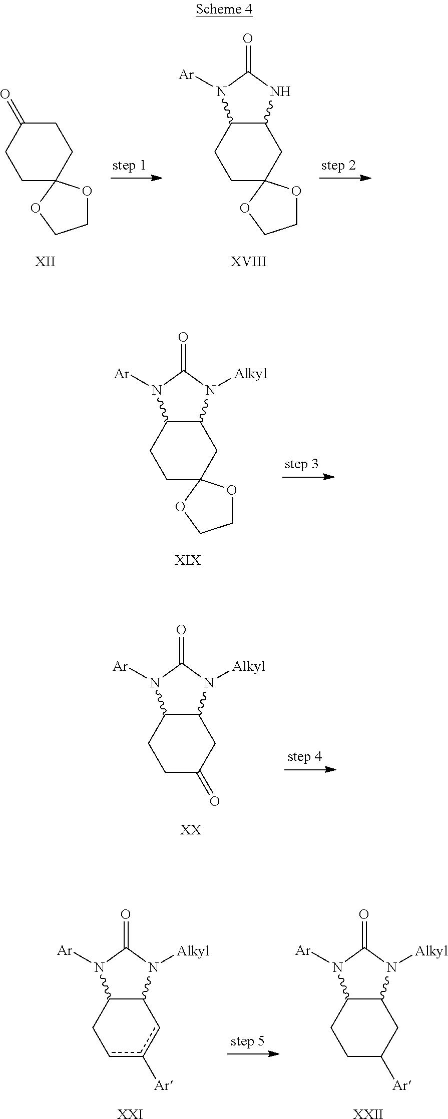

Alkylated ureas of formula XIX could be obtained corresponding ureas of formula XVIII by treating with suitable alkylating agent such as alkyl bromide, alkyl mesilate or alkyl tosylate in presence of base such as NaH, KOtBu, in suitable solvent such as DMF. Compounds of formula XVIII could be synthesized from compounds of formula XII as mentioned previously in schemes 1 and 2. Compounds of formula XX could be obtained as mentioned in scheme 3 using standard deprotecting conditions. Compounds of formula) XXI could be synthesized from compounds of formula XX by using Grignard reaction with suitable arylmagnesium halide followed by elimination reaction of the tertiary alcohol under acidic conditions. Compounds of formula XXI could also be synthesized via the corresponding enol-triflates. Treating ketones of formula XX with triflic anhydride in presence of base such as triethyl amine can give corresponding enol trifleates which in turn can be converted into compounds of formula XXI by standard Suzuki coupling reaction. Compounds of formula XXII can be easily obtained by reduction of the double bond by known standard reactions.

### Methods of Use

The compounds of formula (I) are androgen receptor (AR) antagonists and are therefore useful in the treatment of diseases associated with AR. Such diseases include prostate cancer, including primary, recurrent and hormone-refractory prostate cancer. Moreover, the compounds of formula (I) may also be useful in the treatment of conditions and diseases such as: male contraception, a variety of male hormone related conditions such as hypersexuality and sexual deviation; benign prostate hyperplasia, acne vugaris, androgenetic alopecia, and hirsutism. The compounds of formula (I) may also be useful in preventing the symptoms associated with reduced testosterone such as hot flashes after castration and in purposefully presenting or counteracting masculinisation in the case of transsexual women undergoing sex reassignment therapy.

The term “a therapeutically effective amount” of a compound of the present invention refers to an amount of a compound of formula (I) that will elicit the biological or medical response of a subject, for example, reduction or inhibition of receptor activity, or ameliorate symptoms, alleviate conditions, slow or delay disease progression, or prevent a disease, etc. In one non-limiting embodiment, the term “a therapeutically effective amount” refers to the amount of a compound of formula (I) when administered to a subject, is effective to (1) at least partially alleviating, inhibiting, preventing and/or ameliorating a condition, or a disorder or a disease (i) mediated by AR or (ii) associated with AR activity, or (iii) characterized by activity (normal or abnormal) of AR; or (2) reducing or inhibiting the AR or (3) reducing or inhibiting the expression of AR. In another non-limiting embodiment, the term “a therapeutically effective amount” refers to the amount of a compound of formula (I) when administered to a cell, or a tissue, or a non-cellular biological material, or a medium, is effective to at least partially reducing or inhibiting the activity of AR; or at least partially reducing or inhibiting the expression of AR.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

The activity of a compound according to the present invention can be assessed by the biological assay given herein.

Thus, as a further embodiment, the present invention provides the use of a compound of formula (I) in therapy. In a further embodiment, the therapy is selected from a disease or condition which is treated by an androgen receptor antagonist. In another embodiment, the disease is prostate cancer, suitably primary prostate cancer or hormone-refractory prostate cancer. In another embodiment the disease or condition is benign prostate hypertrophy. In another embodiment the condition is a male hormone related condition such as hypersexuality and sexual deviation. In another embodiment the disease or condition is acne vugaris, androgenetic alopecia, or hirsutism.

In another embodiment, the invention provides a use of a compound of formula (I) in that manufacture of a medicament for the treatment of a disease or condition mediated by AR inhibition. In a further embodiment, the disease or condition is one which is treated by an androgen receptor antagonist. In another embodiment, the disease is prostate cancer, suitably primary prostate cancer or hormone-refractory prostate cancer. In another embodiment the disease or condition is benign prostate hypertrophy. In another embodiment the condition is a male hormone related condition such as hypersexuality and sexual deviation. In another embodiment the disease or condition is acne vugaris, androgenetic alopecia, or hirsutism.

In another embodiment, the invention provides a method for the treatment of a disease or condition mediated by AR inhibition comprising administration of a therapeutically effective amount of a compound of formula (I) to a subject in need thereof. In a further embodiment, the disease or condition is one which is treated by an androgen receptor antagonist. In another embodiment, the disease is prostate cancer, suitably primary prostate cancer or hormone-refractory prostate cancer. In another embodiment the disease or condition is benign prostate hypertrophy. In another embodiment the condition is a male hormone related condition such as hypersexuality and sexual deviation. In another embodiment the disease or condition is acne vugaris, androgenetic alopecia, or hirsutism.

### Compositions

In another aspect, the present invention provides a pharmaceutical composition comprising a compound of formula (I), or a pharmaceutically acceptable salt thereof, and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.
Tablets may be either film coated or enteric coated according to methods known in the art.

Suitable compositions for oral administration include an effective amount of a compound of formula (I), or a pharmaceutically acceptable salt thereof, in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurized container, pump, spray, atomizer or nebuliser, with or without the use of a suitable propellant.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The pharmaceutical composition or combination of the present invention can be in a unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

### Combinations

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound of formula (I) and at least one other therapeutic agent as a combined preparation for simultaneous, separate or sequential use in therapy. In one embodiment, the therapy is the treatment of a disease or condition mediated by androgen receptor. Products provided as a combined preparation include a composition comprising the compound of formula (I) and the other therapeutic agent(s) together in the same pharmaceutical composition, or the compound of formula (I) and the other therapeutic agent(s) in separate form, e.g. in the form of a kit.

In one embodiment, the invention provides a pharmaceutical composition comprising a compound of formula (I) and another therapeutic agent(s). Optionally, the pharmaceutical composition may comprise a pharmaceutically acceptable excipient, as described above.

In one embodiment, the invention provides a kit comprising two or more separate pharmaceutical compositions, at least one of which contains a compound of formula (I).

In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of a compound of formula (I) for treating a disease or condition mediated by androgen receptor wherein the medicament is prepared for administration with another therapeutic agent. The invention also provides the use of another therapeutic agent for treating a disease or condition mediated by androgen receptor, wherein the medicament is administered with a compound of formula (I).

The invention also provides a compound of formula (I) for use in a method of treating a disease or condition mediated by androgen receptor wherein the compound of formula (I) is prepared for administration with another therapeutic agent. The invention also provides another therapeutic agent for use in a method of treating a disease or condition mediated by androgen receptor, wherein the other therapeutic agent is prepared for administration with a compound of formula (I). The invention also provides a compound of formula (I) for use in a method of treating a disease or condition mediated by androgen receptor, wherein the compound of formula (I) is administered with another therapeutic agent. The invention also provides another therapeutic agent for use in a method of treating a disease or condition mediated by androgen receptor, wherein the other therapeutic agent is administered with a compound of formula (I).

The invention also provides the use of a compound of formula (I) for treating a disease or condition mediated by androgen receptor wherein the patient has previously (e.g. within 24 hours) been treated with another therapeutic agent. The invention also provides the use of another therapeutic agent for treating a disease or condition mediated by androgen receptor, wherein the patient has previously (e.g. within 24 hours) been treated with a compound of formula (I).

In one embodiment the other therapeutic agent is selected from the group consisting of: hormone therapy agents such as GnRH agonists; androgen receptor antagonists: inhibitors of oncogenic kinases, e.g. VEGF, mTOR, EGFR, CYP17 and PI3K; cancer chemotherapy agents such as taxanes, topoisomerase II inhibitors, and anti-tumor antibiotics; HSP90 inhibitors, agents or natural extracts known to promote hair growth; agents or natural extracts known to treat acne; and agents or natural extracts know to treat hirsutism.

Examples of gonadotropin-releasing hormone (GnRH) receptor agonists include, but are not limited to, leuprolide and leuprolide acetate (sold under the tradenames Viadure® by Bayer AG, Eligard® by Sanofi-Aventis and Lupron® by Abbott Lab).

Examples of androgen receptor antagonists include m but are not limited to, Nilutamide (sold under the tradenames Nilandron® and Anandron®), bicalutamide (sold under tradename Casodex®), flutamide (sold under the tradename Fulexin™), and MDV3100 also known as 4-(3-(4-cyano-3-(trifluoromethyl)phenyl)-5,5-dimethyl-4-oxo-2-thioxoimidazolidin-1-yl)-2-fluoro-N-methylbenzamide.

Examples of Vascular Endothelial Growth Factor (VEGF) receptor inhibitors include, but are not limited to, bevacizumab (sold under the trademark Avastin® by Genentech/Roche), axitinib, (N-methyl-2-[[3-[(E)-2-pyridin-2-ylethenyl]-1H-indazol-6-yl]sulfanyl]benzamide, also known as AG013736, and described in PCT Publication No. WO 01002369), Brivanib Alaninate ((S)—((R)-1-(4-(4-Fluoro-2-methyl-1H-indol-5-yloxy)-5-methylpyrrolo[2,1-f][1,2,4]triazin-6-yloxy)propan-2-yl)-2-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide, and described in PCT Publication No. WO 02066470), pasireotide (also known as SOM230, and described in PCT Publication No. WO 02010192), and sorafenib (sold under the tradename Nexavar®).

Examples of mTOR inhibitors include, but are not limited to, temsirolimus (sold under the tradename Torisel® by Pfizer), ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383), and everolimus (sold under the tradename Afinitor® by Novartis).

Examples of epidermal growth factor receptor (EGFR) inhibitors include, but are not limited to, gefitnib (sold under the tradename Iressa®), N-[4-[(3-Chloro-4-fluorophenyl)amino]-7-[[(3″S″)-tetrahydro-3-furanyl]oxy]-6-quinazolinyl]-4(dimethylamino)-2-butenamide, sold under the tradename Tovok® by Boehringer Ingelheim), cetuximab (sold under the tradename Erbitux® by Bristol-Myers Squibb), and panitumumab (sold under the tradename Vectibix® by Amgen).

Examples of PI3K inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730), and 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/22806).

Examples of cytochrome P450 17A1 (CYP17) inhibitors include, but are not limited to, abiraterone (tradename Zytiga®), galeterone, and orteronel.

Examples of topoisomerase II inhibitors include, but are not limited to, etoposide (also known as VP-16 and Etoposide phosphate, sold under the tradenames Toposar®, VePesid® and Etopophos®), and teniposide (also known as VM-26, sold under the tradename Vumon®).

Examples of taxane anti-neoplastic agents include, but are not limited to, cabazitaxel (1-hydroxy-7β,10β-dimethoxy-9-oxo-5β,20-epoxytax-11-ene-2α,4,13α-triyl-4-acetate-2-benzoate-13-[(2R,3S)-3-{[(tert-butoxy)carbonyl]amino}-2-hydroxy-3-phenylpropanoate), and larotaxel ((2α,3ξ,4α,5β,7α,10β,13α)-4,10-bis(acetyloxy)-13-({(2R,3S)-3-[(tert-butoxycarbonyl)amino]-2-hydroxy-3-phenylpropanoyl}oxy)-1-hydroxy-9-oxo-5,20-epoxy-7,19-cyclotax-11-en-2-yl benzoate).

Examples of anti-tumor antibiotics include, but are not limited to, doxorubicin (sold under the tradenames Adriamycin® and Rubex®), bleomycin (sold under the tradename Lenoxane®), daunorubicin (also known as dauorubicin hydrochloride, daunomycin, and rubidomycin hydrochloride, sold under the tradename Cerubidine®), daunorubicin liposomal (daunorubicin citrate liposome, sold under the tradename DaunoXome®), mitoxantrone (also known as DHAD, sold under the tradename Novantrone®), epirubicin (sold under the tradename Ellence™), idarubicin (sold under the tradenames Idamycin®, Idamycin PFS®), and mitomycin C (sold under the tradename Mutamycin®).

### EXAMPLES

Abbreviations used are those conventional in the art or the following:
C Celsius
CDI 1,1′-carbonyldiimidazole
d doublet
dd doublet of doublets
DCM dichloromethane
DHT dihydrotestosterone
DIPEA N,N-diisopropylethylamine
DMEM Dulbecco's modified eagle's medium
DMF dimethylformamide
DMSO dimethylsulfoxide
EDCI 1-ethyl-3-(3-dimethylaminopropyl)carbodiimide
EtOAc ethyl acetate
FSB fetal bovine serum
g gram
h hour(s)
HATU 2-(1H-7-Azabenzotriazol-1-yl)-1,1,3,3-tetramethyluronium hexafluorophosphate
HPLC high pressure liquid chromatography
IR infrared spectroscopy
kg kilogram
L liter
LCMS liquid chromatography and mass spectrometry
MS mass spectrometry
MW microwave
m multiplet
min minutes
mL milliliter(s)
μM micromolar
m/z mass to charge ratio
nm nanometer
nM nanomolar
N normal
NBS N-bromosuccinimide
NMR nuclear magnetic resonance
Pd(dba)₃ tris(dibenzylideneacetone)dipalladium(0)
RT room temperature
s singlet
triplet
TFA trifluoroacetic acid
THF tetrahydrofuran
TLC thin layer chromatography
   The following examples are intended to be illustrative only and not limiting in any way.

### Intermediate 1: Trans-4-[(2-aminocyclohexyl)amino]-2-(trifluoromethyl)benzonitrile (±)

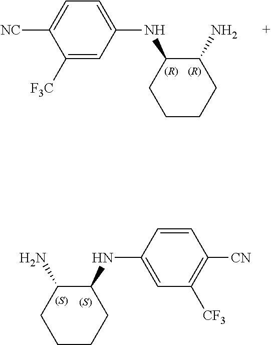

To a solution of trans-1,2-diaminocyclohexane [racemic(±)] (0.50 g, 4.39 mmol) in DMSO (10 mL) under Ar atmosphere was added 4-fluoro-2-(trifluoromethyl)benzonitrile (0.83 g, 4.39 mmol) at RT and the resulting reaction mixture was heated to 45° C. After stirring for 2 h, the reaction mixture was cooled to RT, poured onto ice-water (20 mL), and extracted with ethyl acetate (50 mL×2). The combined organic layer was washed with water (50 mL) followed by brine (50 mL), dried over Na₂SO₄, and concentrated under reduced pressure to give a residue. The crude residue was triturated with ether (5 mL×2) to give the title compound (0.300 g, 24.2%) as a white solid. LCMS: m/z 284.3 [M+H]⁺.

### Intermediate 2: Trans-4-(2-oxooctahydro-1H-benzo[c]imidazol-1-yl)-2-(trifluoromethyl)benzonitrile (±)

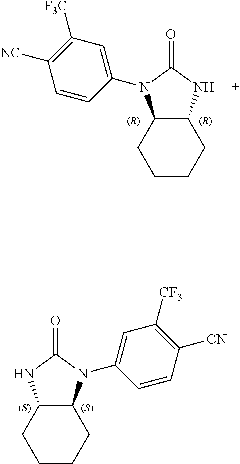

To a solution of trans-4-[(2-aminocyclohexyl)amino]-2-(trifluoromethyl)benzonitrile [racemic(±)] (0.500 g, 1.76 mmol) in THF (10 mL) was added triethylamine (0.74 mL, 5.29 mmol) followed by 1,1′-carbonyldiimidazole (0.572 g, 3.53 mmol) at RT under N₂ atmosphere. After stirring for 12 h, the reaction mixture was quenched by adding water (10 mL), and concentrated under reduced pressure. The aqueous layer was further diluted with water (50 mL) and extracted with ethyl acetate (50 mL×2). The combined organic layer was washed with water (50 mL) followed by brine (50 mL), dried over Na₂SO₄, and concentrated under reduced pressure to give a residue. The crude residue was purified by column chromatography on silica gel (dichloromethane/methanol=991) to give the title compound (0.240 g, 45.0%) as a white solid. LCMS: m/z 310 [M+H]⁺; ¹H NMR (400 MHz, CDCl₃) δ 7.81 (d, 1H), 7.70 (d, 1H), 7.55 (dd, 1H), 4.98 (s, 1H), 4.65 (ddd, 1H), 3.33 (ddd, 1H), 2.29-2.26 (m, 1H), 2.17-2.10 (m, 1H), 1.94 (d, 2H), 1.64-1.39 (m, 4H).

### Intermediate 3: 2-Fluoro-4-iodo-N-methylbenzamide

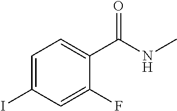

To a suspension of 4-amino-2-fluoro-N-methylbenzamide (20.0 g, 118.9 mmol) in 5N HCl (200 mL) was added a solution of NaNO₂ (12.3 g, 178.4 mmol) in water (80 mL) at 0° C. The reaction mixture was stirred for 30 min at the same temperature. A solution of Kl (43.4 g, 261.5 mmol) in water (80 mL) was added slowly to the above reaction mixture over a period of 20 min at 0° C. The resulting reaction mixture was allowed to warm to RT and stirred for 1 h. The reaction mixture was neutralized with 5N NaOH and extracted with ethyl acetate (150 mL×3). The combined organic layer was washed with water (100 mL×2), dried over Na₂SO₄, and concentrated under reduced pressure to give a residue.

The residue was purified by column chromatography on silica gel (hexanes/ethyl acetate=31) to give the title compound (27.0 g, 81.8%) as a white solid. ¹H NMR (400 MHz, CDCl₃) δ 7.82 (t, 1H), 7.62 (d, 1H), 7.51 (d, 1H), 6.74-6.62 (bs, 1H), 3.02 (d, 3H).

### Example 1

### Trans-4-{3-[4-cyano-3-(trifluoromethyl)phenyl]-2-oxo-octahydro-1H-1,3-benzodiazol-1-yl}-2-fluoro-N-Methylbenzamide (±)

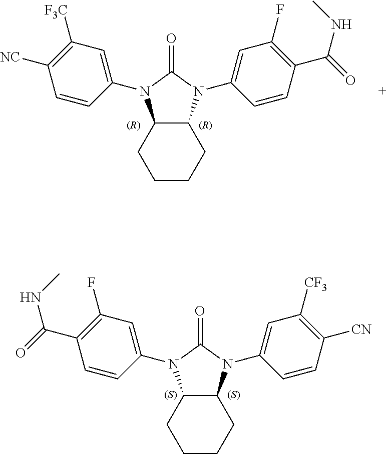

A suspension of trans-4-(2-oxooctahydro-1H-benzo[d]imidazol-1-yl)-2-(trifluoromethyl)-benzonitrile [racemic(±)] (0.20 g, 0.65 mmol), 2-fluoro-4-iodo-N-methylbenzamide (0.18 g, 0.65 mmol), trans-1,2-diaminocyclohexane (±) (0.022 g, 0.03 mmol) and tripotassium phosphate (0.866 g, 1.94 mmol) in toluene (10 mL) was degassed for 30 min in a microwave vial. CuI (0.006 g, 0.03 mmol) was added and the vial was sealed with an aluminum cap. The sealed vial was kept in a preheated oil bath at 110° C. and stirred for 12 h. The reaction mixture was cooled to RT, filtered through a pad of celite, and the filtrate was concentrated under reduced pressure to give a residue. The residue was purified by column chromatography on silica gel (dichloromethane/methanol=982) to give the title compound (0.090 g, 30.3%) as a white solid. HPLC: 95.2%; LCMS: m/z 461 [M+H]⁺; ¹H NMR (400 MHz, CDCl₃) δ 8.19 (t, 1H), 7.86 (d, 1H), 7.76 (d, 1H), 7.56 (dd, 1H), 7.18 (dd, 1H), 7.09 (dd, 1H) 6.74-6.68 (m, 1H), 3.76-3.73 (m, 2H), 3.06 (d, 3H), 2.44-2.39 (m, 2H), 2.06 (d, 2H), 1.66-1.52 (m, 4H). The enantiomeric mixture was separated by preparative Chiral HPLC to give example 1a Trans-4-{3-[4-cyano-3-(trifluoromethyl)phenyl]-2-oxo-octahydro-1H-1,3-benzodiazol-1-yl}-2-fluoro-N-Methylbenzamide (+) [0.037 g, retention time: 4.183 min, [α_{D}²⁵=+86 (c=0.105, MeOH), HPLC: 95.47%] and example 1 b Trans-4-{3-[4-cyano-3-(trifluoromethyl)phenyl]-2-oxo-octahydro-1H-1,3-benzodiazol-1-yl}-2-fluoro-N-Methylbenzamide (−) [0.045 g, retention time: 5.536 min, [α]_{D}²⁵=86 (c=0.106, MeOH), HPLC: 99.32%] as white solids.

Method: Column: LUXAMYLOSE; Mobile phase: Heptane (A)/Ethanol (B); Isocratic: 50:50: A: B; Flow: 20 mL/min.

### Example 2

### Trans-4,4′-(2-oxohexahydro-1H-benzo[d]imidazole-1,3(2H)-diyl)bis(2-(trifluoromethyl)benzonitrile) (±)

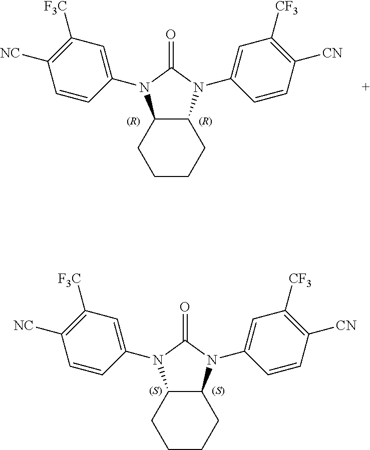

Trans-4-(2-oxooctahydro-1H-benzo[d]imidazol-1-yl)-2-(trifluoromethyl)benzonitrile [racemic(±)] (0.300 g, 0.97 mmol) was reacted with 4-iodo-2-(trifluoromethyl)benzonitrile (0.29 g, 0.97 mmol) as described for the synthesis of example 1 to give a residue. The residue was purified by column chromatography on silica gel (dichloromethane/methanol=1000 to 973) to afford the title compound (0.20 g, 43.1%) as a white solid. HPLC: 94.74%; ¹H NMR (400 MHz, CDCl₃) δ 7.89 (d, 2H), 7.72 (s, 2H), 7.55 (d, 2H), 3.81 (m, 2H), 2.42 (d, 2H), 2.11 (d, 2H), 1.65-1.50 (m, 4H). The enantiomeric mixture was separated by preparative Chiral HPLC to give example 2a Trans-4,4′-(2-oxohexahydro-1H-benzo[d]imidazole-1,3(2H)-diyl)bis(2-(trifluoromethyl)benzonitrile) (−) [0.080 g, retention time: 9.749 min, [α]_{D}²⁵=114 (c=0.116, MeOH), HPLC: 96.58%] and example 2b Trans-4,4′-(2-oxohexahydro-1H-benzo[d]imidazole-1,3(2H)-diyl)bis(2-(trifluoromethyl)benzonitrile) (+) [0.095 g, retention time: 20.238 min, [α]_{D}²⁵=+117 (c=0.10, MeOH), HPLC: 98.99%] as white solids.

Method: Column: CHIRALPAK AD-H (20 mm×250 mm×5 u); Mobile phase: n-hexane:ethanol::70:30 (isocratic); Flow: 20 mL/min.

### Example 3

### Trans-4-(3-(furan-3-yl)-2-oxoocta-hydro-1H-benzo[d]imidazol-1-yl)-2-trifluoromethyl)benzonitrile (±)

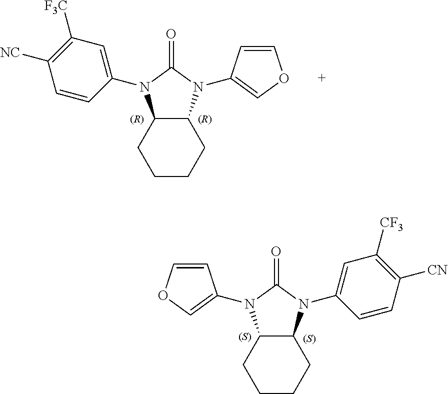

Trans-4-(2-oxooctahydro-1H-benzo[d]imidazol-1-yl)-2-(trifluoromethyl)benzonitrile [racemic(±)] (0.150 g, 0.49 mmol) was reacted with 3-bromofuran (0.071 g, 0.49 mmol) as described for the synthesis of example 1 to give a residue. The residue was purified by column chromatography on silica gel (dichloromethane/methanol=1000 to 973) to give the title compound (0.03 g, 16.5%) as a white solid. HPLC: 95.67%; LCMS: m/z 376 [M+H]⁺; ¹H NMR (400 MHz, CDCl₃) δ 7.83 (d, 1H), 7.76 (d, 1H), 7.56-7.52 (m, 2H), 7.40 (t, 1H), 6.59 (d, 1H), 3.68 (ddd, 1H), 3.45 (ddd, 1H), 2.34 (d, 2H), 2.04-2.02 (m, 2H), 1.60-1.42 (m, 4H).

### Example 4

### Trans-4-(2-oxo-3-(pyridin-4-yl)octahydro-1H-benzo[d]imidazol-1-yl)-2-(trifluoromethyl)benzonitrile (±)

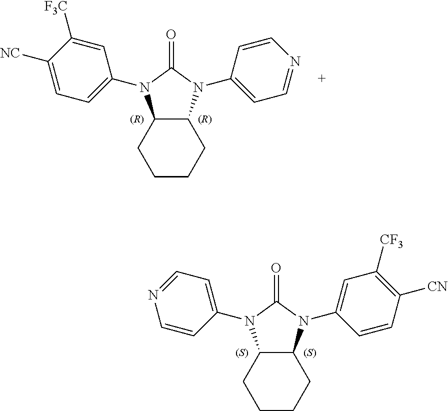

Trans-4-(2-oxooctahydro-1H-benzo[d]imidazol-1-yl)-2-(trifluoromethyl)benzonitrile [racemic(±)] (0.100 g, 0.32 mmol) was reacted with 4-bromopyridine (0.05 g, 0.32 mmol) as described for the synthesis of example 1 to give a residue. The residue was purified by column chromatography on silica gel (dichloromethane/methanol/Et₃N=1000 to 9730.2 mL) to give the title compound (0.020 g, 16.0%) as a white solid. HPLC: 95.03%; LCMS: m/z 387.3 [M+H]⁺; ¹H NMR (400 MHz, CDCl₃) δ 8.61-8.60 (m, 2H), 7.78 (d, 1H), 7.74 (d, 1H), 7.55 (dd, 1H), 7.22 (m, 2H), 3.75 (m, 2H), 2.46 (dd, 2H), 2.08 (d, 2H), 1.64-1.52 (m, 4H).

### Example 5

### Trans-4-(2-oxo-3-phenyloctahydro-1H-benzo[d]imidazol-1-yl)-2-(trifluoromethyl)benzonitrile (±)

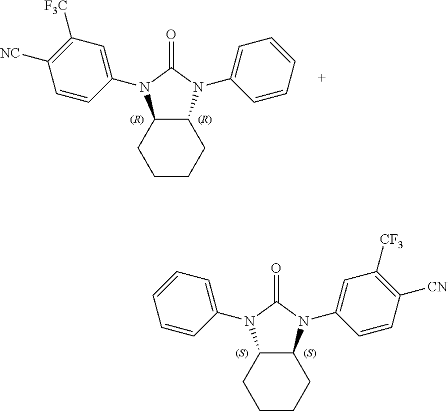

Trans-4-(2-oxooctahydro-1H-benzo[d]imidazol-1-yl)-2-(trifluoromethyl)benzonitrile [racemic(±)] (0.100 g, 0.32 mmol) was reacted with 4-iodobenzene (0.066 g, 0.32 mmol) as described for the synthesis of example 1 to give a residue. The residue was purified by column chromatography on silica gel (dichloromethane/methanol=1000 to 973) to give the title compound (0.035 g, 28.1%) as a white solid. HPLC: 97.64%; LCMS: m/z 386.1 [M+H]⁺; ¹H NMR (400 MHz, CDCl₃) δ 8.85 (d, 2H), 7.55 (dd, 1H), 7.42 (t, 2H), 7.26-7.23 (m, 3H), 3.75-3.66 (m, 2H), 2.40 (d, 1H), 2.31 (d, 1H), 2.04-1.99 (m, 2H), 1.64-1.49 (m, 4H). The enantiomeric mixture was separated by preparative Chiral HPLC to give example 5a Trans-4-(2-oxo-3-phenyloctahydro-1H-benzo[d]imidazol-1-yl)-2-(trifluoromethyl)benzonitrile (+) [0.005 g, retention time: 12.643 min, [α]_{D}²⁵=+49 (c=0.05, MeOH), HPLC: 96.36%] and example 5b Trans-4-(2-oxo-3-phenyloctahydro-1H-benzo[d]imidazol-1-yl)-2-(trifluoromethyl)benzonitrile (−) [0.010 g, retention time: 20.028 min., [α]_{D}²⁵=23 (c=0.04, MeOH), HPLC: 96.68%] as white solids. Column: LUXAMYLOSE-2; Mobile phase: n-hexane:ethanol:: 80:20 (isocratic); Flow: 20 mL/min.

### Example 6

### Trans-ethyl-4-(3-(4-cyano-3-trifluoromethyl)phenyl)-2-oxooctahydro-1H-benzo[d]imidazol-1-yl)-2-fluorobenzoate (±)

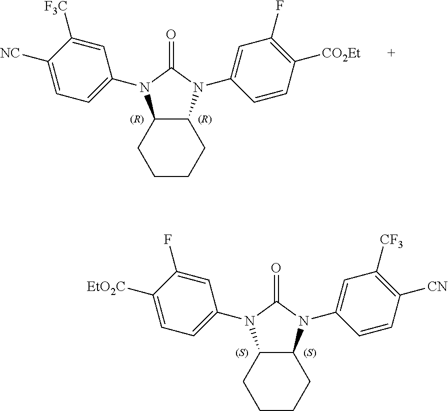

Trans-4-(2-oxooctahydro-1H-benzo[d]imidazol-1-yl)-2-(trifluoromethyl)benzonitrile [racemic(±)] (0.700 g, 2.27 mmol) was reacted with ethyl 2-fluoro-4-iodobenzoate (0.66 g, 2.27 mmol) as described for the synthesis of example 1 to give a residue. The residue was purified by column chromatography on silica gel (dichloromethane/methanol=1000 to 973) to give the title compound (0.62 g, 57.6%) as a white solid. HPLC: 98.2%; LCMS: m/z 476.1 [M+H]⁺; ¹H NMR (400 MHz, CDCl₃) δ 7.99 (t, 1H), 7.84 (d, 1H), 7.74 (d, 1H), 7.55 (dd, 1H), 7.10 (ddd, 2H), 4.40 (q, 2H), 3.74 (ddd, 2H), 2.40 (ddd, 2H), 2.07 (d, 2H), 1.63-1.51 (m, 4H), 1.38 (t, 3H).

### Example 7

### Trans-ethyl-4-{3-[4-cyano-3-(trifluoromethyl)phenyl]-2-oxo-octahydro-1H-1,3-benzodiazol-1-yl}-2-methylbenzoate (±)

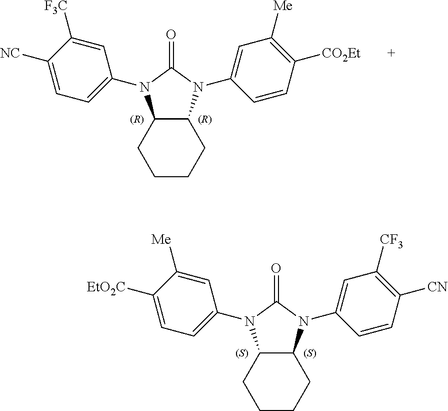

Trans-4-(2-oxooctahydro-1H-benzo[d]imidazol-1-yl)-2-(trifluoromethyl)benzonitrile [racemic(±)] (0.30 g, 0.97 mmol) was reacted with ethyl 4-bromo-2-methylbenzoate (0.236 g, 0.97 mmol) as described for the synthesis of example 1 to give a residue. The residue was purified by column chromatography on silica gel (dichloromethane/methanol=1000 to 982) to give the title compound (0.200 g, 43.7%) as a white solid. LCMS: m/z 472.1 [M+H]⁺; ¹H NMR (400 MHz, CDCl₃) δ 7.99 (d, 1H), 7.84 (d, 1H), 7.76 (d, 1H), 7.55 (dd, 1H), 7.16 (d, 1H), 7.13 (dd, 1H), 4.30 (q, 3H), 3.77-3.72 (m, 2H), 2.63 (s, 2H), 2.38 (t, 2H), 2.04 (d, 2H), 1.65-1.60 (m, 2H), 1.57-1.49 (m, 2H), 1.39 (t, 3H).

### Example 8

### Trans-ethyl-5-(3-(4-cyano-3-(trifluoromethyl)phenyl)-2-oxooctahydro-1H-benzo[d]imidazol-1-yl)thiophene-2-carboxylate (±)

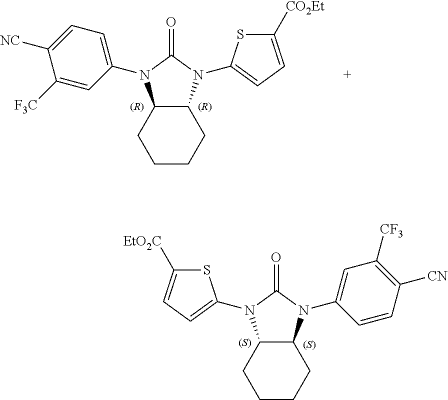

Trans-4-(2-oxooctahydro-1H-benzo[d]imidazol-1-yl)-2-(trifluoromethyl)benzonitrile [racemic(±)] (0.300 g, 0.97 mmol) was reacted with ethyl 5-bromothiophene-2-carboxylate (0.228 g, 0.97 mmol) as described for the synthesis of example 1 to give a residue. The residue was purified by column chromatography on silica gel (dichloromethane/methanol=1000 to 982) to give the title compound (0.17 g, 37.8%) as a white solid. HPLC: 95%; LCMS: m/z 464 [M+H]⁺; ¹H NMR (400 MHz, CDCl₃) δ 7.86 (d, 1H), 7.75 (d, 1H), 7.67 (d, 1H), 7.56 (dd, 1H), 6.78 (d, 1H), 4.36 (q, 2H), 3.76 (ddd, 1H), 3.63 (ddd, 1H), 2.63 (d, 1H), 2.38 (d, 1H), 2.10-2.04 (m, 2H), 1.69-1.49 (m, 4H), 1.36 (t, 3H).

### Example 9

### Trans-6-(3-(4-cyano-3-(trifluoromethyl)phenyl)-2-oxo octahydro-1H-benzo[c]imidazol-1-yl) pyridine-2-sulfonamide (±)

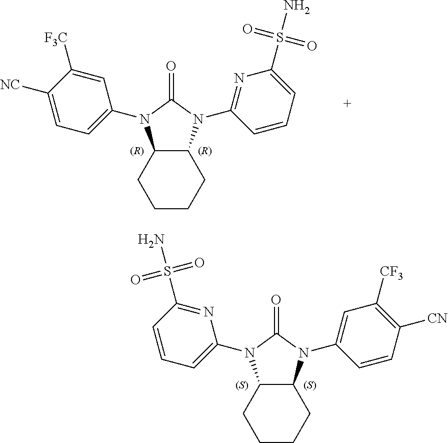

Trans-4-(2-oxooctahydro-1H-benzo[d]imidazol-1-yl)-2-(trifluoromethyl)benzonitrile [racemic(±)] (0.200 g, 0.65 mmol) was reacted with N,N-dibenzyl-6-bromopyridine-2-sulfonamide (0.270 g, 0.65 mmol) as described for the synthesis of example 1 to give a residue. The residue was purified by column chromatography on silica gel (dichloromethane/methanol=1000 to 982) to give trans-N,N-dibenzyl-6-{3-[4-cyano-3-(trifluoromethyl)phenyl]-2-oxo-octahydro-1H-1,3-benzodiazol-1-yl}pyridine-2-sulfonamide (±) (0.30 g, 71.8%) as a white solid. LCMS: m/z 646.1 [M+H]⁺; ¹H NMR (400 MHz, CDCl₃) δ 7.94-7.79 (m, 4H), 7.76 (d, 1H), 7.56 (dd, 1H), 7.26-7.21 (m, 6H), 7.11-7.08 (m, 4H), 4.57 (q, 4H), 3.68 (ddd, 1H), 3.50 (ddd, 1H), 2.70 (d, 1H), 2.33 (d, 1H), 2.00-1.90 (m, 2H), 1.45-1.30 (m, 4H). To a solution of trans-N,N-dibenzyl-6-{3-[4-cyano-3-(trifluoromethyl)phenyl]-2-oxo-octahydro-1H-1,3-benzodiazol-1-yl}pyridine-2-sulfonamide [racemic(±)] (0.300 g, 0.46 mmol) in dichloromethane (5 mL) was added conc. H₂SO₄ (1 mL) at 0° C. and the resulting reaction mixture was allowed to warm to RT. After stirring for 30 min, ice-water (10 mL) was added and extracted with dichloromethane (25 mL×3). The combined organic layer was washed with saturated NaHCO₃ solution (20 mL×3) followed by water (20 mL), dried over Na₂SO₄, and concentrated under reduced pressure to give a residue. The residue was purified by column chromatography on silica gel (dichloromethane/methanol=1000 to 982) to give the title compound (0.100 g, 46.2%) as a white solid. HPLC: 93.88%; LCMS: m/z 466.1 [M+H]⁺; ¹H NMR (400 MHz, CDCl₃) δ 7.95-7.90 (m, 2H), 7.87 (d, 1H), 7.81-7.78 (m, 1H), 7.75 (d, 1H), 7.56-7.53 (m, 1H), 4.95 (s, 2H), 3.98 (ddd, 1H), 3.80-3.74 (m, 1H), 2.97 (dd, 1H), 2.37 (dd, 1H), 2.17-2.00 (m, 2H), 1.70-1.45 (m, 4H).

### Example 10

### Trans-4-(3-(2-fluoropyridin-4-yl)-2-oxooctahydro-1H-benzo[d]imidazol-1-yl)-2-(trifluoromethyl)benzonitrile (±)

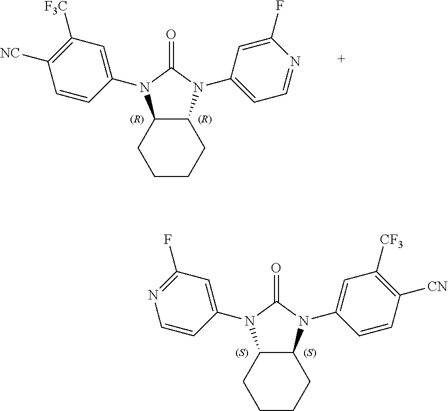

Trans-4-(2-oxooctahydro-1H-benzo[d]imidazol-1-yl)-2-(trifluoromethyl)benzonitrile [racemic(±)] (0.100 g, 0.32 mmol) was reacted with 2-fluoro-4-iodopyridine (0.072 g, 0.32 mmol) as described for the synthesis of example 1 to give a residue. The residue was purified by column chromatography on silica gel (dichloromethane/methanol=1000 to 982) to give the title compound (0.035 g, 26.8%) as a white solid. HPLC: 94.59%; LCMS: m/z 405.1 [M+H]⁺; ¹H NMR (400 MHz, CDCl₃) δ 8.21 (d, 1H), 7.87 (d, 1H), 7.70 (d, 1H), 7.55 (dd, 1H), 7.17-7.15 (m, 1H), 6.85 (d, 1H), 3.79-3.74 (m, 2H), 2.52 (d, 1H), 2.40 (d, 1H), 2.10-2.07 (m, 2H), 1.67-1.56 (m, 4H). The enantiomeric mixture was separated by preparative chiral HPLC to give example 10a Trans-4-(3-(2-fluoropyridin-4-yl)-2-oxooctahydro-1H-benzo[d]imidazol-1-yl)-2-(trifluoromethyl)benzonitrile (+) [0.005 g, retention time: 6.927 min., [α]_{D}²⁵=+78 (c=0.056, MeOH), HPLC: 94.93%] and example 10b Trans-4-(3-(2-fluoropyridin-4-yl)-2-oxooctahydro-1H-benzo[d]imidazol-1-yl)-2-(trifluoromethyl)benzonitrile (−) [0.012 g, retention time: 10.64 min., [α]_{D}²⁵=50 (c=0.044, MeOH), HPLC: 94.64%] as white solid. Column: LUXAMYLOSE-2 AXIA PACKED; Mobile phase=heptane:ethanol::60:40: (isocratic); Flow: 20 mL/min.

### Example 11

### Trans-4-(2-oxo-3-(2-(trifluoromethyl)pyridin-4-yl)octahydro-1H-benzo[d]imidazol-1-yl)-2-(trifluoromethyl)benzonitrile (±)

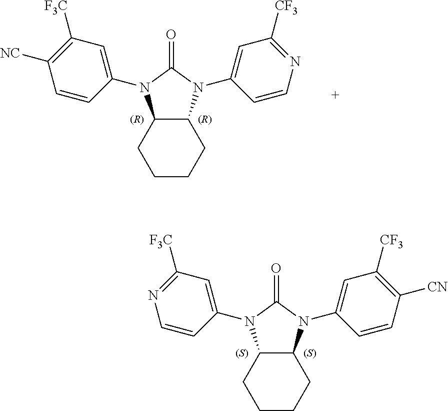

Trans-4-(2-oxooctahydro-1H-benzo[d]imidazol-1-yl)-2-(trifluoromethyl)benzonitrile [racemic(±)] (0.200 g, 0.65 mmol) was reacted with 4-iodo-2-(trifluoromethyl)pyridine (0.146 g, 0.65 mmol) as described for the synthesis of example 1 to give a residue. The residue was purified by column chromatography on silica gel (dichloromethane/methanol=1000 to 982) to give the title compound (0.025 g, 17.0%) as a white solid. HPLC: 94.43%; LCMS: m/z 455.1 [M+H]⁺; ¹H NMR (400 MHz, CDCl₃) δ 8.71 (d, 1H), 7.88 (d, 1H), 7.73 (d, 1H), 7.63 (d, 1H), 7.57-7.54 (m, 1H), 7.41-7.39 (m, 1H), 3.82-3.79 (m, 2H), 2.50 (d, 1H), 2.40 (d, 1H), 2.11-2.09 (m, 2H), 1.69-1.52 (m, 4H).

### Example 12

### Trans-N-(4-(3-(4-cyanophenyl)-2-oxooctahydro-1H-benzo[d]imidazol-1-yl)-2-fluorophenyl)acetamide (±)

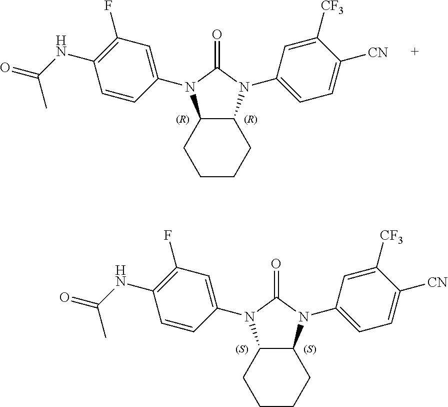

Trans-4-(2-oxooctahydro-1H-benzo[d]imidazol-1-yl)-2-(trifluoromethyl)benzonitrile [racemic(±)] (0.140 g, 0.45 mmol) was reacted with N-(2-fluoro-4-iodophenyl)acetamide (0.130 g, 0.45 mmol) as described for the synthesis of example 1 to give a residue. The residue was purified by column chromatography on silica gel (hexanes/ethyl acetate=11) to give the title compound (0.07 g, 34%) as a pale yellow solid. LCMS: m/z 461 [M+H]⁺; ¹H NMR (300 MHz, CDCl₃) δ 8.34 (t, 1H), 7.83 (d, 1H), 7.75 (d, 1H), 7.45 (dd, 1H), 7.34 (bs, 1H), 7.11 (dd, 1H), 6.95 (dd, 1H), 3.60-3.76 (m, 2H), 2.25-2.42 (m, 2H), 2.23 (s, 3H), 2.10-1.98 (m, 2H), 1.66-1.44 (m, 4H).

### Example 13

### Trans-N-(4-(3-(4-cyano-3-(trifluoromethyl)phenyl)-2-oxooctahydro-1H-benzo[d]imidazol-1-yl)-2-fluorophenyl)-N-methylacetamide (±)

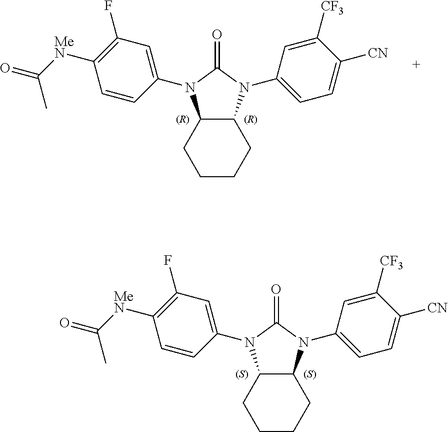

Trans-4-(2-oxooctahydro-1H-benzo[d]imidazol-1-yl)-2-(trifluoromethyl)benzonitrile [racemic(±)] (0.093 g, 0.3 mmol) was reacted with N-(2-fluoro-4-iodophenyl)-N-methylacetamide (0.088 g, 0.3 mmol) as described for the synthesis of example 1 to give a residue. The residue was purified by column chromatography on silica gel (hexanes/ethyl acetate=64) to give the title compound (0.060 g, 42%) as a white solid. LCMS: m/z 475 [M+H]⁺; ¹H NMR (300 MHz, CDCl₃) δ 7.85 (d, 1H), 7.77 (s, 1H), 7.54 (dd, 1H), 7.31-7.25 (m, 1H), 7.12 (t, 2H), 3.80-3.60 (m, 2H), 3.22 (s, 3H), 2.38-2.41 (m, 2H), 2.05-2.08 (m, 2H), 1.90 (s, 3H), 1.50-1.70 (m, 4H).

### Example 14

### Trans-4-(3-(3-fluoro-4-(methylamino)phenyl)-2-oxooctahydro-1H-benzo[d]im-idazol-1-yl)-2-(trifluoromethyl)benzonitrile (±)

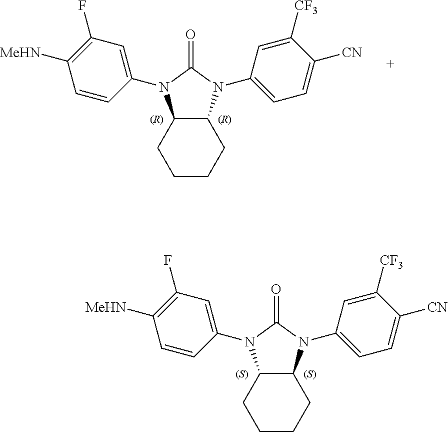

Trans-N-(4-(-3-(4-cyano-3-(trifluoromethyl)phenyl)-2-oxooctahydro-1H-benzo[d]imidazol-1-yl)-2-fluorophenyl)-N-methylacetamide [racemic(±)] (0.03 g, 0.06 mmol) was added in conc. HCl (4.0 mL) and the reaction mixture was refluxed for 24 h. The reaction mixture was cooled to RT, neutralized with saturated NaHCO₃, extracted with CHCl₃ (15 mL×3), dried over Na₂SO₄, and concentrated under reduced pressure to give a residue. The residue was purified by column chromatography on silica gel (hexanes/ethyl acetate=11) to give the title compound (0.080 g, 32%) as a white solid. LCMS: m/z 433 [M+H]⁺; ¹H NMR (400 MHz, CDCl₃) δ 7.75-7.90 (m, 2H), 7.57 (s, 1H), 6.92 (d, 2H), 6.69 (s, 1H), 3.99 (bs, 1H), 3.67 (bs, 1H), 3.52 (bs, 1H), 2.90 (s, 3H), 2.25-2.0 (m, 4H), 1.60-1.40 (m, 4H).

### Example 15

### Trans-(4-(-3-(4-amino-3-fluorophenyl)-2-oxooctahydro-1H-benzo[d]imidazol-1-yl)-2-(trifluoromethyl)benzonitrile (±)

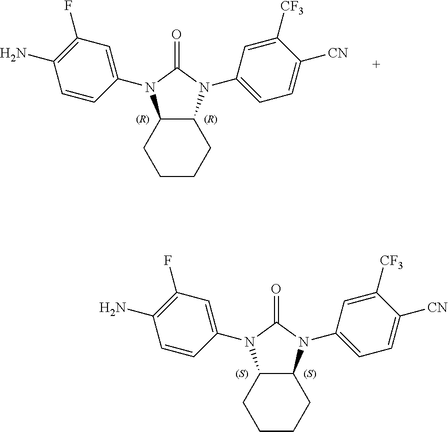

Trans-4-(2-oxooctahydro-1H-benzo[d]imidazol-1-yl)-2-(trifluoromethyl)benzonitrile [racemic(±)] (0.600 g, 1.93 mmol) was reacted with 2-fluoro-4-iodoaniline (0.459 g, 1.93 mmol) as described for the synthesis of example 1 to give a residue. The residue was purified by column chromatography on silica gel (hexanes/ethyl acetate=11) to give the title compound (0.400 g, 50%) as a white solid. LCMS: m/z 419 [M+H]⁺; ¹H NMR (400 MHz, CDCl₃) δ 7.8\tilde{3}7.77 (m, 2H), 7.55 (d, 1H), 6.92 (d, 1H), 6.82-6.77 (m, 2H), 3.76 (bs, 2H), 3.67 (t, 1H), 3.52 (t, 1H), 2.37 (d, 1H), 2.17 (d, 1H), 2.10-1.95 (m, 2H), 1.60-1.45 (m, 4H).

### Example 16

### Trans-N-(4-(3-(4-cyano-3-(trifluoromethyl)phenyl)-2-oxooctahydro-1H-benzo[d]imidazol-1-yl)-2-fluorophenyl)methanesulfonamide (±)

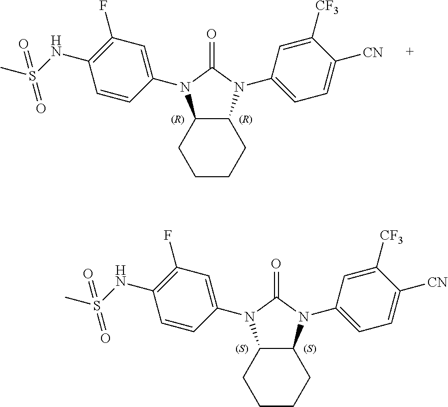

To a solution of trans-4-(3-(4-amino-3-fluorophenyl)-2-oxooctahydro-1H-benzo[d]imidazol-1-yl)-2-(trifluoromethyl)benzonitrile [racemic(±)] (0.100 g, 0.239 mmol) in CH₂Cl₂ (20 mL) was added pyridine (0.028 g, 0.359 mmol) followed by MsCl (0.030 g, 0.262 mmol) at 0° C. After stirring for 16 h at RT, the reaction mixture was diluted with CH₂Cl₂ (10 mL), and washed with saturated NaHCO₃ (20 mL×2) followed by brine (20 mL×2). The organic layer was separated, dried over Na₂SO₄, and concentrated under reduced pressure to give a residue. The residue was purified by column chromatography on silica gel (hexanes/ethyl acetate=5545) to give the title compound (0.030 g, 25.3%) as a white solid. HPLC=97.08%; LCMS: m/z 496.8 [M+H]⁺; ¹H NMR (400 MHz, CDCl₃) δ 7.85 (d, 1H), 7.77 (s, 1H), 7.62 (t, 1H), 7.55 (dd, 1H), 7.18 (dd, 1H), 7.02 (d, 1H), 6.47 (s, 1H), 3.74 (dd, 1H), 3.66 (t, 1H), 3.05 (s, 3H), 2.40 (d, 1H), 2.33 (d, 1H), 2.05 (bs, 2H), 1.65-1.51 (m, 4H).

### Example 17

### Trans-4-(3-(4-cyano-3-(trifluoromethyl)phenyl)-2-oxooctahydro-1H-benzo[d]im-idazol-1-yl)-2-fluorobenzoic acid (±)

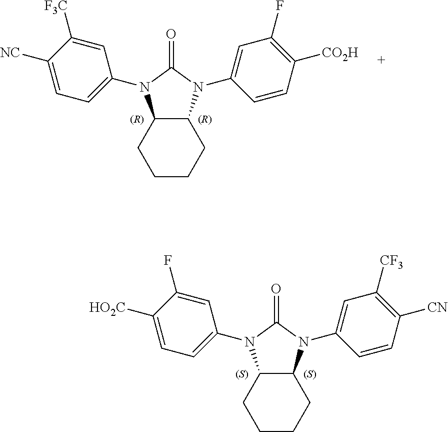

To a solution of trans-ethyl 4-(3-(4-cyano-3-trifluoromethyl)phenyl)-2-oxooctahydro-1H-benzo[d]imidazol-1-yl)-2-fluorobenzoate [racemic(±)] (0.300 g, 0.63 mmol) in MeOH (10 mL) was added aqueous solution of sodium hydroxide (0.028 g, 0.69 mmol, 2 mL) at RT and the resulting reaction mixture was stirred for 12 h. The reaction mixture was concentrated under reduced pressure to give a residue. The residue was dissolved in water (25 mL) and extracted with diethyl ether (25 mL×2). The pH of aqueous layer was adjusted to 2 using conc. HCl and extracted with ethyl acetate (50 mL×2). The combined organic layer was washed with water (50 mL) followed by brine (50 mL), dried over Na₂SO₄, and concentrated under reduced pressure to give a residue. The residue was triturated with ether (5 mL×2) to give the title compound (0.210 g, 74.5%) as a white solid. HPLC: 95.45%; LCMS: m/z 448.2 [M+H]⁺; ¹H NMR (400 MHz, DMSO-d₆) δ 13.2 (bs, 1H), 8.20 (d, 1H), 7.96 (d, 1H), 7.91 (d, 1H), 7.77 (dd, 1H), 7.33-7.25 (m, 2H), 4.03-3.87 (m, 2H), 2.33-2.27 (m, 2H), 1.91-1.85 (m, 2H), 1.64-1.59 (m, 2H), 1.44-1.39 (m, 2H).

### Example 18

### Trans-4-(3-(4-cyano-3-(trifluoromethyl)phenyl)-2-oxooctahydro-1H-benzo[d]im-idazol-1-yl)-2-fluoro-N-methoxybenzamide (±)

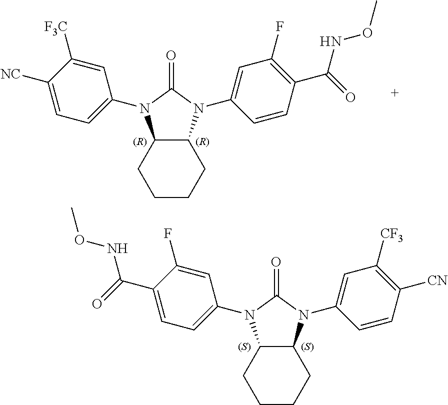

To a solution of trans-4-(3-(4-cyano-3-(trifluoromethyl)phenyl)-2-oxooctahydro-1H-benzo[d]imidazol-1-yl)-2-fluorobenzoic acid [racemic(±)] (0.05 g, 0.11 mmol) and O-methyl hydroxylamine hydrochloride (0.028 g, 0.34 mmol) in DMF (10 mL) was added triethylamine (0.062 mL, 0.45 mmol) followed by HATU (0.127 g, 0.34 mmol) at RT under N₂ atmosphere. After stirring for 16 h, the reaction mixture was diluted with cold water (30 mL) and extracted with ethyl acetate (50 mL×2). The combined organic layer was washed with 2N HCl (50 mL×3) followed by 10% NaHCO₃ (50 mL×2) and brine (50 mL). The organic layer was dried over Na₂SO₄ and concentrated under reduced pressure to give a residue. The residue was purified by preparative TLC (hexanes/ethyl acetate=6040) to afford the title compound (0.025 g, 46.9%) as a white solid. HPLC: 96.04%; LCMS: m/z 477.0 [M+H]⁺; ¹H NMR (400 MHz, CDCl₃) δ 9.26 (d, 1H), 8.17 (t, 1H), 7.86 (d, 1H), 7.74 (d, 1H), 7.54 (dd, 1H), 7.21-7.17 (m, 1H), 7.13-7.10 (m, 1H), 3.91 (s, 3H), 3.78-3.72 (m, 2H), 2.46-2.36 (m, 2H), 2.10-2.00 (m, 2H), 1.66-1.55 (m, 4H).

### Example 19

### Trans-4-(3-(4-cyano-3-(trifluoromethyl)phenyl)-2-oxooctahydro-1H-benzo[d]im-idazol-1-yl)-N-(cyclopropylmethoxy)-2-fluorobenzamide (±)

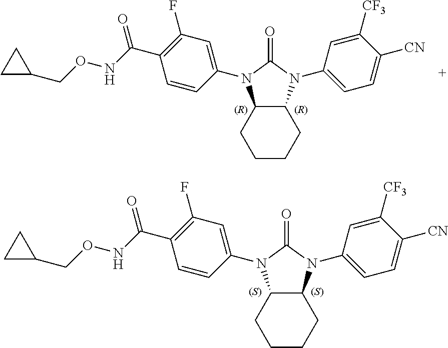

To a solution of trans-4-(3-(4-cyano-3-(trifluoromethyl)phenyl)-2-oxooctahydro-1H-benzo[d]imidazol-1-yl)-2-fluorobenzoic acid [racemic(±)] (0.100 g, 0.223 mmol) and 0-(cyclopropyl/methyl) hydroxylamine hydrochloride (0.030 g, 0.246 mmol) in DMF (3 mL) was added DIPEA (0.11 mL, 0.669 mmol) followed by EDCI (0.047 g, 0.246 mmol) and HOBT (0.036 g, 0.267 mmol) at 0° C. under N₂ atmosphere. The reaction mixture was allowed to warm to RT and stirred for 16 h. The reaction mixture was diluted with DCM (50 mL), washed with 1N HCl (25 mL×2) followed by satd NaHCO₃ (25 mL×2) and brine (25 mL×1). The organic layer was dried over Na₂SO₄ and concentrated to give a residue. The residue was purified by column chromatography on silica gel (dichloromethane/methanol=991) to give the title compound (0.030 g, 26%) as a white solid. HPLC: 93.43%; LCMS: m/z 517 [M+H]⁺; ¹H NMR (400 MHz, DMSO-d₆) δ 11.44 (s, 1H), 8.24 (d, 1H), 7.99 (d, 1H), 7.80 (dd, 1H), 7.65 (t, 1H), 7.34 (dd, 1H), 7.27 (dd, 1H), 4.02 (ddd, 1H), 3.91 (ddd, 1H), 3.75 (d, 2H), 2.33 (m, 2H), 1.89 (d, 2H), 1.64 (t, 2H), 1.44 (m, 2H), 1.29 (m, 1H), 0.57 (q, 2H), 0.32 (q, 2H).

### Example 20

### Trans-4-(3-(4-cyano-3-(trifluoromethyl)phenyl)-2-oxooctahydro-1H-benzo[d]im-idazol-1-yl)-2-fluoro-N-(2-methoxy ethoxy)benzamide (±)

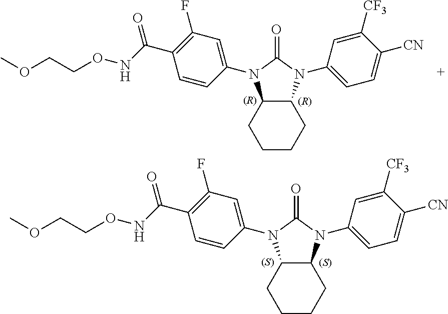

Trans-4-(3-(4-cyano-3-(trifluoromethyl)phenyl)-2-oxooctahydro-1H-benzo[d]imidazol-1-yl)-2-fluorobenzoic acid [racemic(±)] (0.100 g, 0.223 mmol) was treated with 0-(2-methoxyethyl)hydroxylamine (0.022 g, 0.246 mmol) as described for the synthesis of example 19 to give a residue. The residue was purified by column chromatography on silica gel (dichloromethane/methanol=991) to give the title compound (0.028 g, 23%) as an off white solid. HPLC: 96.95%; LCMS: m/z 521 [M+H]⁺; ¹H NMR (400 MHz, DMSO-d₆) δ 11.6 (s, 1H), 8.24 (d, 1H), 7.99 (d, 1H), 7.80 (dd, 1H), 7.66 (t, 1H), 7.35 (dd, 1H), 7.27 (dd, 1H), 4.05 (t, 2H), 4.02 (ddd, 1H), 3.91 (ddd, 1H), 3.61 (t, 2H), 3.33 (s, 3H), 2.33 (m, 2H), 1.89 (d, 2H), 1.64 (t, 2H), 1.44 (m, 2H).

### Example 21

### Trans-4-(3-(4-cyano-3-(trifluoromethyl)phenyl)-2-oxooctahydro-1H-benzo[c]im-idazol-1-yl)-N-(cyclobutylmethoxy)-2-fluorobenzamide (±)

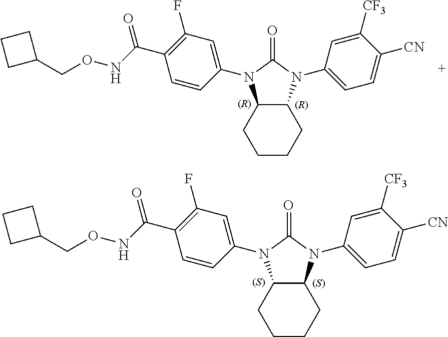

Trans-4-(3-(4-cyano-3-(trifluoromethyl)phenyl)-2-oxooctahydro-1H-benzo[d]imidazol-1-yl)-2-fluorobenzoic acid [racemic(±)] (0.080 g, 0.178 mmol) was treated with 0-(cyclobutylmethyl)hydroxylamine (0.019 g, 0.196 mmol) as described for the synthesis of example 19 to give a residue. The residue was purified by column chromatography on silica gel (dichloromethane/methanol=991) to give the title compound (0.035 g, 37%) as a white solid. HPLC: 97.26%; LCMS: m/z 531 [M+H]⁺; ¹H NMR (400 MHz, CDCl₃) δ 9.11 (d, 1H), 8.15 (t, 1H), 7.85 (d, 1H), 7.73 (s, 1H), 7.53 (dd, 1H), 7.16 (dd, 1H), 7.10 (dd, 1H), 4.04 (d, 2H), 3.74 (ddd, 2H), 2.75 (m, 1H), 2.40 (t, 2H), 2.09 (dd, 4H), 1.87 (m, 4H), 1.60 (m, 4H).

### Example 22

### Trans-4-(3-(4-cyano-3-(trifluoromethyl)phenyl)-2-oxooctahydro-1H-benzo[d]im-idazol-1-yl)-2-fluoro-N-isobutoxybenzamide (±)

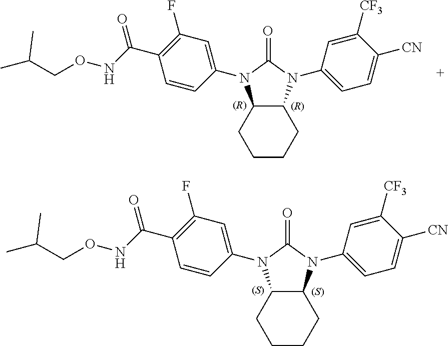

Trans-4-(3-(4-cyano-3-(trifluoromethyl)phenyl)-2-oxooctahydro-1H-benzo[d]imidazol-1-yl)-2-fluorobenzoic acid [racemic(±)] (0.080 g, 0.178 mmol) was treated with 0-isobutylhydroxylamine (0.017 g, 0.196 mmol) as described for the synthesis of example 19 to give a residue. The residue was purified by column chromatography on silica gel (dichloromethane/methanol=99.5/0.5) to give the title compound (0.030 g, 39%) as a white solid. HPLC: 97.16%; LCMS: m/z 519 [M+H]⁺; ¹H NMR (400 MHz, CDCl₃) δ 9.13 (d, 1H), 8.15 (t, 1H), 7.85 (d, 1H), 7.73 (s, 1H), 7.54 (dd, 1H), 7.16 (dd, 1H), 7.10 (dd, 1H), 3.83 (d, 2H), 3.74 (ddd, 2H), 2.40 (t, 2H), 2.06 (m, 1H), 2.04 (d, 2H), 1.62 (m, 2H), 1.53 (m, 2H), 1.00 (d, 6H).

### Example 23

### Trans-N-((1-(tert-butoxy)propan-2-yl)oxy)-4-(3-(4-cyano-3-(trifluoromethyl)phenyl)-2-oxooctahydro-1H-benzo[d]imidazol-1-yl)-2-fluorobenzamide (±)

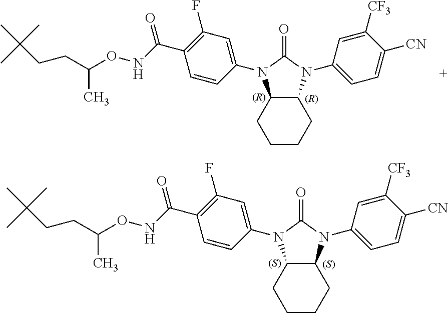

Trans-4-(3-(4-cyano-3-(trifluoromethyl)phenyl)-2-oxooctahydro-1H-benzo[d]imidazol-1-yl)-2-fluorobenzoic acid [racemic(±)] (0.080 g, 0.178 mmol) was treated with O-(1-(tert-butoxy)propan-2-yl)hydroxylamine (0.029 g, 0.196 mmol) as described for the synthesis of example 19 to give a residue. The residue was purified by column chromatography on silica gel (dichloromethane/methanol=99.5/0.5) to give the title compound (0.012 g, 11.6%) as a white solid. HPLC: 96.84%; LCMS: m/z 578 [M+H]⁺; ¹H NMR (400 MHz, CDCl₃) δ 10.08 (d, 1H), 8.17 (t, 1H), 7.85 (d, 1H), 7.73 (d, 1H), 7.54 (dd, 1H), 7.16 (dd, 1H), 7.10 (dd, 1H), 4.19 (m, 1H), 3.73 (ddd, 2H), 3.54 (d, 2H), 2.40 (t, 2H), 2.05 (d, 2H), 1.61 (m, 2H), 1.52 (m, 2H), 1.32 (d, 3H), 1.24 (s, 9H).

### Example 24

### Trans-4-(3-(4-cyano-3-(trifluoromethyl)phenyl)-2-oxooctahydro-1H-benzo[c]im-idazol-1-yl)-2-fluoro-N-((1-hydroxypropan-2-yl)oxy)benzamide (±)

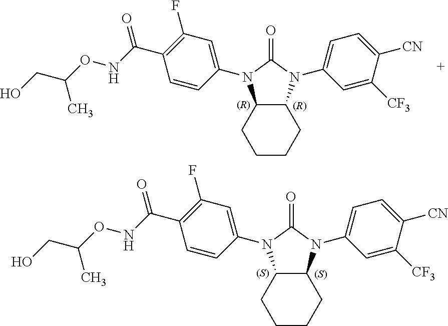

To a solution of trans-N-((1-(tert-butoxy)propan-2-yl)oxy)-4-(3-(4-cyano-3-(trifluoromethyl)phenyl)-2-oxooctahydro-1H-benzo[d]imidazol-1-yl)-2-fluorobenzamide (0.100 g, 0.173 mmol) in DCM (30 mL) was added TFA (0.13 mL, 1.73 mmol) at RT. After stirring for 16 h, excess TFA (0.07 mL, 0.800 mmol) was added at RT, and stirring continued for another 7 h. The reaction mixture was diluted with DCM (10 mL) and washed with satd NaHCO₃ (25 mL×3) followed by brine (25 mL×3). The organic layer was dried over Na₂SO₄ and concentrated under reduced pressure to give a residue. The residue was purified by preparative TLC (hexanes/ethyl acetate=37) to give the title compound (0.030 g, 33.2%) as a white solid. HPLC=98.77%; LCMS: m/z 520.8 [M+H]⁺; ¹H NMR (400 MHz, CDCl₃) δ 9.14 (dd, 1H), 8.16 (t, 1H), 7.86 (d, 1H), 7.74 (d, 1H), 7.55 (dd, 1H), 7.23 (dt, 1H), 7.11 (dt, 1H), 4.43 (bs, 1H), 4.18-4.10 (m, 1H), 3.79-3.68 (m, 3H), 3.55-3.48 (m, 1H), 2.42 (t, 2H), 2.07 (d, 2H), 1.48-1.68 (m, 4H), 1.33 (d, 3H).

### Example 25

### Trans 4-(3-(4-cyano-3-(trifluoromethyl)phenyl)-2-oxooctahydro-1H-benzo[d]im-idazol-1-yl)-N-(3-(dimethylamino)propoxy)-2-fluorobenzamide (±)

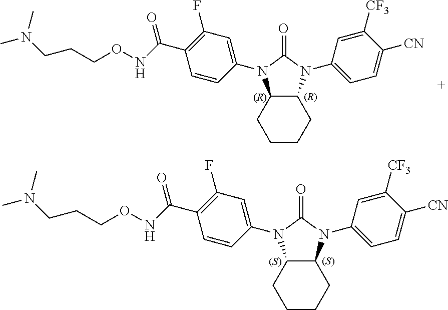

Trans-4-(3-(4-cyano-3-(trifluoromethyl)phenyl)-2-oxooctahydro-1H-benzo[d]imidazol-1-yl)-2-fluorobenzoic acid [racemic(±)] (0.080 g, 0.178 mmol) was treated with 3-(aminooxy)-N,N-dimethylpropan-1-amine (0.023 g, 0.196 mmol) as described for the synthesis of example 19 to give a residue. The residue was purified by column chromatography on silica gel (dichloromethane/methanol=98.5/1.5) to give the title compound (0.018 g, 18%) as an off white solid. HPLC: 97.85%; LCMS: m/z 548 [M+H]⁺; ¹H NMR (400 MHz, CDCl₃) δ 8.06 (t, 1H), 7.85 (d, 1H), 7.74 (d, 1H), 7.54 (dd, 1H), 7.13 (dd, 1H), 7.08 (dd, 1H), 4.12 (t, 2H), 3.73 (ddd, 2H), 2.52 (bs, 2H), 2.40 (d, 2H), 2.31 (s, 6H), 2.04 (m, 4H), 1.52 (m, 4H)

### Example 26

### Trans-4-(3-(4-cyano-3-(trifluoromethyl)phenyl)-2-oxooctahydro-1H-benzo[d]im-idazol-1-yl)-N-(2-(diethylamino)ethoxy)-2-fluorobenzamide (±)

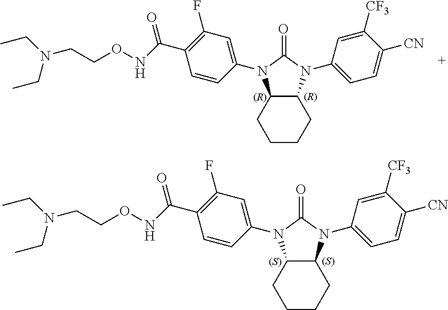

Trans-4-(3-(4-cyano-3-(trifluoromethyl)phenyl)-2-oxooctahydro-1H-benzo[d]imidazol-1-yl)-2-fluorobenzoic acid [racemic(±)] (0.080 g, 0.178 mmol) was treated with 2-(aminooxy)-N,N-diethylethanamine (0.026 g, 0.196 mmol) as described for the synthesis of example 19 to give a residue. The residue was purified by column chromatography on silica gel (dichloromethane/methanol=98.5/1.5) to give the title compound (0.022 g, 22.6%) as an off white solid. HPLC: 98.81%; LCMS: m/z 562 [M+H]⁺; ¹H NMR (400 MHz, CDCl₃) δ 8.13 (t, 1H), 7.85 (d, 1H), 7.74 (d, 1H), 7.54 (dd, 1H), 7.16 (dd, 1H), 7.08 (dd, 1H), 4.12 (t, 2H), 3.73 (t, 2H), 2.84 (t, 2H), 2.66 (q, 4H), 2.40 (d, 2H), 2.06 (d, 2H), 1.61 (m, 2H), 1.52 (m, 2H), 1.07 (t, 6H)

### Example 27

### Trans 4-(3-(4-cyano-3-(trifluoromethyl)phenyl)-2-oxooctahydro-1H-benzo[c]im-idazol-1-yl)-2-fluoro-N-((tetrahydrofuran-2-yl)methoxy)benzamide (±)

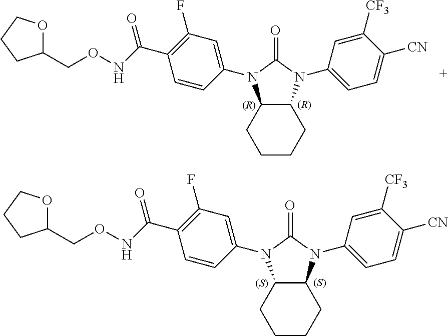

Trans-4-(3-(4-cyano-3-(trifluoromethyl)phenyl)-2-oxooctahydro-1H-benzo[d]imidazol-1-yl)-2-fluorobenzoic acid [racemic(±)] (0.100 g, 0.223 mmol) was treated with 0-((tetrahydrofuran-2-yl)methyl)hydroxylamine (0.054 g, 0.268 mmol) as described for the synthesis of example 19 to give a residue. The residue was purified by column chromatography on silica gel (dichloromethane/methanol=991) to give the title compound (0.028 g, 23%) as a white solid. HPLC: 94.14%; LCMS: m/z 547 [M+H]⁺; ¹H NMR (400 MHz, CDCl₃) δ 9.91 (t, 1H), 8.16 (t, 1H), 7.86 (d, 1H), 7.75 (s, 1H), 7.55 (dd, 1H), 7.16 (dd, 1H), 7.10 (dd, 1H), 4.26 (q, 1H), 4.19 (dd, 1H), 3.95 (q, 2H), 3.86 (q, 1H), 3.76 (dd, 2H), 2.41 (t, 2H), 2.06 (d, 2H), 1.98 (m, 4H), 1.68 (m, 2H), 1.52 (m, 2H).

### Example 28

### Trans 4-(3-(4-cyano-3-(trifluoromethyl)phenyl)-2-oxooctahydro-1H-benzo[c]im-idazol-1-yl)-2-fluoro-N-((1-methylpiperidin-4-yl)oxy)benzamide (±)

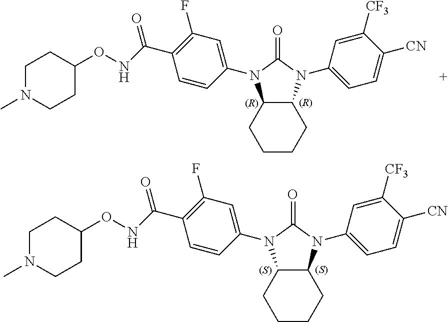

Trans-4-(3-(4-cyano-3-(trifluoromethyl)phenyl)-2-oxooctahydro-1H-benzo[d]imidazol-1-yl)-2-fluorobenzoic acid [racemic(±)] (0.100 g, 0.223 mmol) was treated with 0-((tetrahydrofuran-2-yl)methyl)hydroxylamine (0.035 g, 0.268 mmol) as described for the synthesis of example 19 to give a residue. The residue was purified by preparative HPLC to give the title compound (0.007 g, 7%) as a brown solid. HPLC: 96.3%; LCMS: m/z 560 [M+H]⁺; ¹H NMR (400 MHz, CDCl₃) δ 8.12 (t, 1H), 7.85 (d, 1H), 7.73 (s, 1H), 7.55 (dd, 1H), 7.18 (dd, 1H), 7.12 (dd, 1H), 4.15 (bs, 1H), 3.75 (ddd, 2H), 2.97 (t, 2H), 2.62 (s, 2H), 2.44 (s, 3H), 2.40 (d, 2H), 2.14 (m, 4H), 2.06 (d, 2H), 1.62 (m, 2H), 1.52 (m, 2H).

Column: X Bridge, C18, 19×150 mm, 5 μm

Mobile phase A: 10 mM Ammonium Acetate in water; B: Acetonitrile; Flow: 15.0 mL/min.

| TIME | % B | FLOW (mL/min) |
|---|---|---|
| 0.0 | 30.0 | 15.0 |
| 2.0 | 40.0 | 15.0 |
| 10.0 | 80.0 | 15.0 |

### Example 29

### 4-((3aS,7aS)-3-(4-Cyano-3-(trifluoromethyl)phenyl)-2-oxooctahydro-1H-benzo[d]imidazol-1-yl)-2-fluoro-N-(2-methoxyethoxy)benzamide

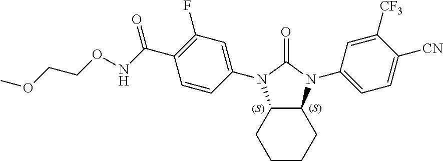

4-((3aS,7aS)-(3-(4-cyano-3-(trifluoromethyl)phenyl)-2-oxooctahydro-1H-benzo[d]imidazol-1-yl)-2-fluorobenzoic acid (Enantiopure) (0.100 g, 0.223 mmol) was reacted with O-(2-methoxyethyl)hydroxylamine (0.025 g, 0.278 mmol) as described for the synthesis of example 19 to give a residue. The residue was purified by column chromatography on silica gel (hexane/ethyl acetate=11) to give the title compound (0.040 g, 34.4%) as a white solid. HPLC=95.27%; LCMS: m/z 520.8 [M+H]⁺; ¹H NMR (400 MHz, CDCl₃) δ 9.65 (d, 1H), 8.16 (t, 1H), 7.86 (d, 1H), 7.74 (d, 1H), 7.55 (dd, 1H), 7.16 (d, 1H), 7.11 (dd, 1H), 4.24-4.20 (m, 2H), 3.78-3.70 (m, 4H), 3.45 (s, 3H), 2.46-2.37 (m, 2H), 2.31-2.23 (m, 2H), 1.68-1.49 (m, 4H).

### Example 30

### Trans-4-(3-(4-cyano-3-(trifluoromethyl)phenyl)-2-oxooctahydro-1H-benzo[d]im-idazol-1-yl)-2-fluorobenzamide (±)

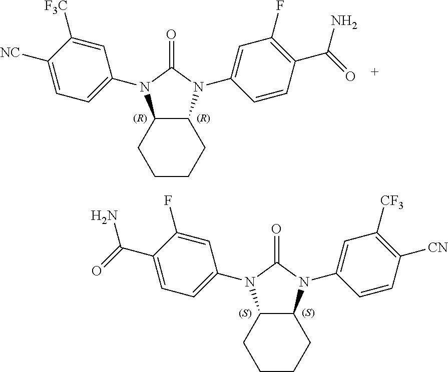

Trans-4-(3-(4-cyano-3-(trifluoromethyl)phenyl)-2-oxooctahydro-1H-benzo[d]imidazol-1-yl)-2-fluorobenzoic acid [racemic(±)] (0.05 g, 0.11 mmol) was reacted with ammonium chloride (0.018 g, 0.34 mmol) as described for the synthesis of example 18 to give a residue. The residue was purified by preparative TLC (hexanes/ethyl acetate=64) to give the title compound (0.012 g, 24.1%) as a white solid. HPLC: 97.34%; LCMS: m/z 447.1 [M+H]⁺; ¹H NMR (400 MHz, CDCl₃) δ 8.18 (t, 1H), 7.86 (d, 1H), 7.75 (d, 1H), 7.55 (dd, 1H), 7.20 (dd, 1H), 7.11-7.09 (m, 1H), 6.68-6.65 (m, 1H), 5.78 (bs, 1H), 3.77-3.74 (m, 2H), 2.45-2.39 (m, 2H), 2.08-2.06 (m, 2H), 1.66-1.52 (m, 4H).

### Example 31

### 4-((3aS,7aS)-3-(4-cyano-3-(trifluoromethyl)phenyl)-2-oxooctahydro-1H-benzo[d]imidazol-1-yl)-2-fluorobenzamide

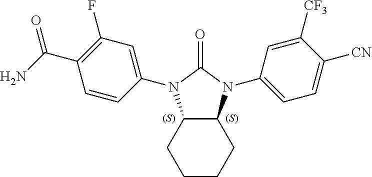

4-((3aS,7aS)-(3-(4-cyano-3-(trifluoromethyl)phenyl)-2-oxooctahydro-1H-benzo[d]imidazol-1-yl)-2-fluorobenzoic acid (Enantiopure) (0.100 g, 0.223 mmol) was reacted with ammonium chloride (0.024 g, 0.448 mmol) as described for the synthesis of example 18 to give a residue. The residue was purified by column chromatography on silica gel (hexanes/ethyl acetate=64) to give the title compound (0.040 g, 40.0%) as a white solid. HPLC=99.58%; LCMS: m/z 446.8 [M+H]⁺; ¹H NMR (400 MHz, CDCl₃) δ 8.18 (t, 1H), 7.86 (d, 1H), 7.75 (d, 1H), 7.55 (dd, 1H), 7.21 (dd, 1H), 7.11 (dd, 1H), 6.66 (d, 1H), 5.78 (s, 1H), 3.80-3.70 (m, 2H), 2.42 (t, 2H), 2.06 (t, 2H), 1.70-1.50 (m, 4H).

### Example 32

### Trans 4-(3-(4-cyano-3-(trifluoromethyl)phenyl)-2-oxooctahydro-1H-benzo[d]im-idazol-1-yl)-2-fluoro-N-(2-hydroxyethyl)benzamide (±)

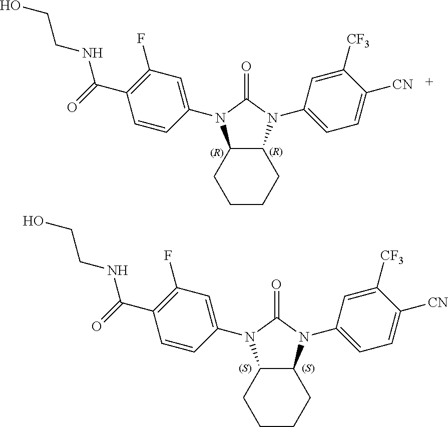

Trans-4-(3-(4-cyano-3-(trifluoromethyl)phenyl)-2-oxooctahydro-1H-benzo[d]imidazol-1-yl)-2-fluorobenzoic acid [racemic (±)] (0.100 g, 0.223 mmol) was treated with 2-aminoethanol (0.016 g, 0.268 mmol) as described for the synthesis of example 18 to give a residue. The residue was purified by column chromatography on silica gel (hexanes/ethyl acetate=64) to give the title compound (0.045 g, 41%) as a white solid. HPLC: 97.4%; LCMS: m/z 491 [M+H]⁺; ¹H NMR (400 MHz, CDCl₃) δ 8.16 (t, 1H), 7.86 (d, 1H), 7.74 (s, 1H), 7.55 (dd, 1H), 7.18 (d, 1H), 7.12 (dd, 1H), 3.85 (d, 2H), 3.75 (dd, 2H), 3.68 (t, 2H), 2.45 (m, 1H), 2.41 (d, 2H), 2.06 (d, 2H), 1.62 (m, 2H), 1.52 (m, 2H).

### Example 33

### 4-((3aS,7aS)-3-(4-cyano-3-(trifluoromethyl)phenyl)-2-oxooctahydro-1H-benzo[d]imidazol-1-yl)-2-fluoro-N-(2-hydroxyethyl)benzamide

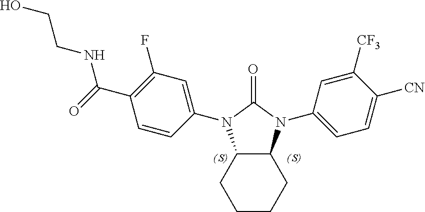

4-((3aS,7aS)-3-(4-cyano-3-(trifluoromethyl)phenyl)-2-oxooctahydro-1H-benzo[d]imidazol-1-yl)-2-fluorobenzoic acid (Enantiopure) (0.220 g, 0.492 mmol) was treated with 2-aminoethanol (0.036 g, 0.590 mmol) as described for the synthesis of example 18 to give a residue. The residue was purified by column chromatography on silica gel (hexanes/ethyl acetate=64) to give the title compound (0.090 g, 37%) as a white solid. HPLC: 97.96%; LCMS: m/z 491 [M+H]⁺; ¹H NMR (400 MHz, DMSO-d₆) δ 8.20 (d, 2H), 7.95 (s, 1H), 7.77 (d, 1H), 7.69 (t, 1H), 7.29 (d, 1H), 7.23 (d, 1H), 4.75 (t, 1H), 3.98 (t, 1H), 3.90 (t, 1H), 3.49 (m, 2H), 3.35 (m, 2H), 2.28 (dd, 2H), 1.85 (d, 2H), 1.61 (t, 2H), 1.39 (d, 2H).

### Example 34

### 4-((3aR,7aR)-3-(4-cyano-3-(trifluoromethyl)phenyl)-2-oxooctahydro-1H-benzo[d]imidazol-1-yl)-2-fluoro-N—((R)-tetrahydrofuran-3-yl)benzamide and 4-((3aS,7aS)-3-(4-cyano-3-(trifluoromethyl)phenyl)-2-oxooctahydro-1H-benzo[d]imidazol-1-yl)-2-fluoro-N—((R)-tetrahydrofuran-3-yl)benzamide

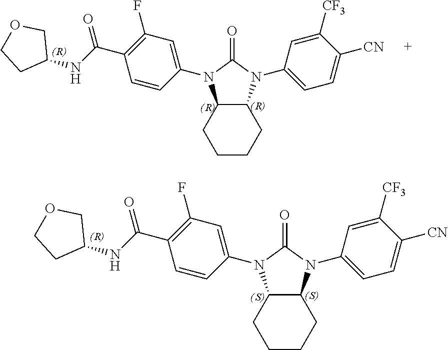

Trans-4-(3-(4-cyano-3-(trifluoromethyl)phenyl)-2-oxooctahydro-1H-benzo[d]imidazol-1-yl)-2-fluorobenzoic acid [racemic (±)] (0.100 g, 0.223 mmol) was treated with (R)-tetrahydrofuran-3-amine (Enantiopure) (0.194 g, 2.23 mmol) as described for the synthesis of example 18 to give a residue. The residue was purified by preparative HPLC to give the title compound (0.021 g, 18%) as a white solid. HPLC: 98.8%; LCMS: m/z 517 [M+H]⁺; ¹H NMR (400 MHz, CDCl₃) δ 8.14 (t, 1H), 7.86 (d, 1H), 7.74 (s, 1H), 7.55 (dd, 1H), 7.18 (d, 1H), 7.10 (dd, 1H), 4.74 (s, 1H), 3.97 (m, 2H), 3.87 (m, 1H), 3.80 (m, 3H), 2.38 (m, 3H), 2.06 (d, 2H), 1.93 (bs, 1H), 1.62 (m, 2H), 1.52 (m, 2H).

Column: TRAIL-250*25 MM

Mobile phase A: 10 MM Ammonium acetate in H₂O; B:1:1 MeOH:ACN; Flow: 30 mL/min.

| TIME | % B | FLOW (mL/MIN) |
|---|---|---|
| 0.0 | 70.0 | 20.0 |
| 2.0 | 70.0 | 20.0 |
| 8.0 | 90.0 | 20.0 |

### Example 35

### 4-((3aS,7aS)-3-(4-cyano-3-(trifluoromethyl)phenyl)-2-oxooctahydro-1H-benzo[d]imidazol-1-yl)-2-fluoro-N—((S)-tetrahydrofuran-3-yl)benzamide and 4-((3aS,7aS)-3-(4-cyano-3-(trifluoromethyl)phenyl)-2-oxooctahydro-1H-benzo[c]imidazol-1-yl)-2-fluoro-N—((R)-tetrahydrofuran-3-yl)benzamide

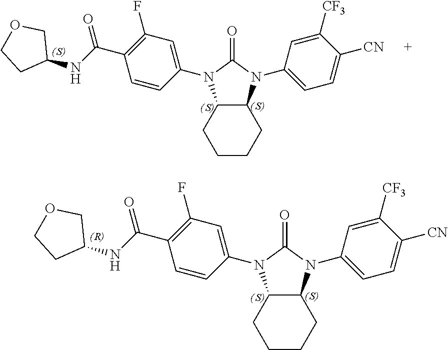

4-((3aS,7aS)-3-(4-cyano-3-(trifluoromethyl)phenyl)-2-oxooctahydro-1H-benzo[d]imidazol-1-yl)-2-fluorobenzoic acid (Enantiopure) (0.500 g, 1.118 mmol) was treated with tetrahydrofuran-3-amine [racemic (±)] (1.02 g, 11.18 mmol) as described for the synthesis of example 18 to give a residue. The residue was purified by preparative HPLC to give the title compound (0.102 g, 16%) as a white solid. HPLC: 98.43%; LCMS: m/z 517 [M+H]⁺; ¹H NMR (400 MHz, CDCl₃) δ 8.14 (t, 1H), 7.86 (d, 1H), 7.74 (s, 1H), 7.55 (dd, 1H), 7.18 (d, 1H), 7.10 (dd, 1H), 6.86 (m, 1H), 4.73 (s, 1H), 3.97 (m, 2H), 3.85 (m, 1H), 3.76 (m, 3H), 2.39 (m, 3H), 2.06 (d, 2H), 1.93 (bs, 1H), 1.62 (m, 2H), 1.52 (m, 2H).

COLUMN: Zorbax XDB, C-18

METHOD: FLOW: 20.0 mL/min; A: 10 mm NH₄OAc in H₂O; B: ACETONITRILE: MeOH

GRADIENT:

| | Time | % B |
|---|---|---|
| 1 | 0.00 | 50.0 |
| 2 | 2.00 | 60.0 |
| 3 | 8.00 | 80.0 |

### Example 36

### 4-((3aS,7aS)-3-(4-cyano-3-(trifluoromethyl)phenyl)-2-oxooctahydro-1H-benzo[d]imidazol-1-yl)-2-fluoro-N—(R)-tetrahydrofuran-3-yl)benzamide

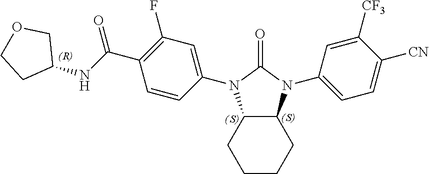

4-((3aS,7aS)-3-(4-cyano-3-(trifluoromethyl)phenyl)-2-oxooctahydro-1H-benzo[d]imidazol-1-yl)-2-fluorobenzoic acid (Enantiopure) (0.200 g, 0.447 mmol) was treated with (R)-tetrahydrofuran-3-amine (Enantiopure) (0.392 g, 4.47 mmol) as described for the synthesis of example 18 to give a residue. The residue was purified by preparative HPLC to give the title compound (0.028 g, 12%) as a white solid. HPLC: 98.67%; LCMS: m/z 517 [M+H]⁺; ¹H NMR (400 MHz, CDCl₃) δ 8.14 (t, 1H), 7.86 (d, 1H), 7.74 (s, 1H), 7.55 (dd, 1H), 7.18 (d, 1H), 7.09 (dd, 1H), 6.86 (m, 1H), 4.73 (s, 1H), 3.97 (m, 2H), 3.85 (m, 1H), 3.76 (m, 3H), 2.39 (m, 3H), 2.06 (d, 2H), 1.93 (bs, 1H), 1.62 (m, 2H), 1.52 (m, 2H).

Column: Zorbax, C18, 21.2×250 mm, 7 μm; Mobile phase A: 10 mM NH₄OAc in H₂O; B:

Acetonitrile; Flow: 20.0 mL/min.

| TIME | % B | FLOW (mL/min) |
|---|---|---|
| 0.0 | 50.0 | 20.0 |
| 2.0 | 60.0 | 20.0 |
| 8.0 | 90.0 | 20.0 |

### Example 37

### 4-((3aS,7aS)-3-(4-cyano-3-(trifluoromethyl)phenyl)-2-oxooctahydro-1H-benzo[d]imidazol-1-yl)-N-(1,3-dihydroxypropan-2-yl)-2-fluorobenzamide

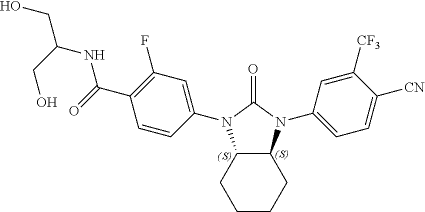

4-((3aS,7aS)-3-(4-cyano-3-(trifluoromethyl)phenyl)-2-oxooctahydro-1H-benzo[d]imidazol-1-yl)-2-fluorobenzoic acid (Enantiopure) (0.100 g, 0.223 mmol) was treated with 2-aminopropane-1,3-diol (0.020 g, 0.246 mmol) as described for the synthesis of example 18 to give a residue. The residue was purified by column chromatography on silica gel (hexanes/ethyl acetate=64) to give the title compound (0.016 g, 13%) as a white solid. HPLC: 98.51%; LCMS: m/z 521 [M+H]⁺; ¹H NMR (400 MHz, DMSO-d₆) δ 8.21 (d, 1H), 7.97 (d, 1H), 7.79 (d, 2H), 7.74 (t, 1H), 7.29 (d, 1H), 7.24 (d, 1H), 4.75 (t, 2H), 3.98 (m, 3H), 3.52 (m, 4H), 2.29 (dd, 2H), 1.85 (d, 2H), 1.61 (t, 2H), 1.39 (d, 2H).

### Example 38

### Trans-4-{3-[4-cyano-3-(trifluoromethyl)phenyl]-2-oxo-octahydro-1H-1,3-benzodiazol-1-yl}-2-methylbenzoic acid (±)

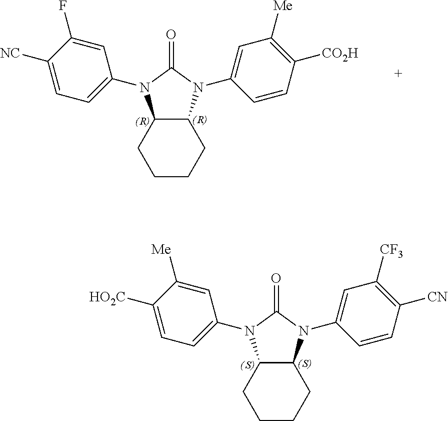

Trans-ethyl-4-{3-[4-cyano-3-(trifluoromethyl)phenyl]-2-oxo-octahydro-1H-1,3-benzodiazol-1-yl}-2-methylbenzoate [racemic(±)] (0.200 g, 0.42 mmol) was treated with sodium hydroxide (0.018 g, 0.47 mmol) as described for the synthesis of example 17 to give a residue. The residue was triturated with ether (5 mL×2) to give the title compound (0.065 g, 34.6%) as a white solid. LCMS: m/z 443.0 [M+H]⁺.

### Example 39

### Trans-4-(3-(4-cyano-3-(trifluoromethyl)phenyl)-2-oxooctahydro-1H-benzo[d]im-idazol-1-yl)-N,2-dimethylbenzamide (±)

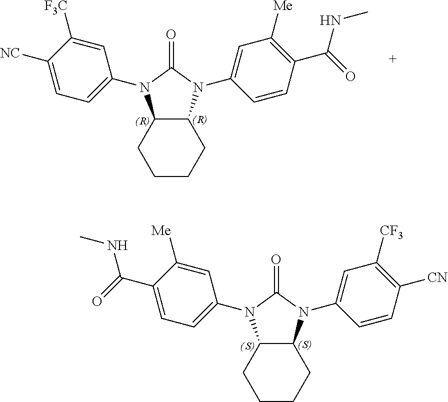

Trans-4-{3-[4-cyano-3-(trifluoromethyl)phenyl]-2-oxo-octahydro-1H-1,3-benzodiazol-1-yl}-2 methylbenzoic acid [racemic(±)] (0.06 g, 0.14 mmol) was reacted with methylamine hydrochloride (0.022 g, 0.41 mmol) as described for the synthesis of example 18 to give a residue. The residue was purified by preparative TLC (hexanes/ethyl acetate=64) to give the title compound (0.030 g, 48.6%) as a white solid. HPLC: 94%; LCMS: m/z 457.1 [M+H]⁺; ¹H NMR (400 MHz, CDCl₃) δ 7.84 (d, 1H), 7.77 (d, 1H), 7.54 (dd, 1H), 7.40 (d, 1H), 7.10-7.06 (m, 2H), 3.73-3.69 (m, 2H), 3.01 (d, 3H), 2.47 (s, 3H), 2.41 (d, 1H), 2.29 (d, 1H), 2.03 (m, 2H), 1.61-1.47 (m, 4H).

### Example 40

### Trans-5-(3-(4-cyano-3-(trifluoromethyl)phenyl)-2-oxooctahydro-1H-benzo[d]imidazol-1-yl)-N-methylthiophene-2-carboxamide (±)

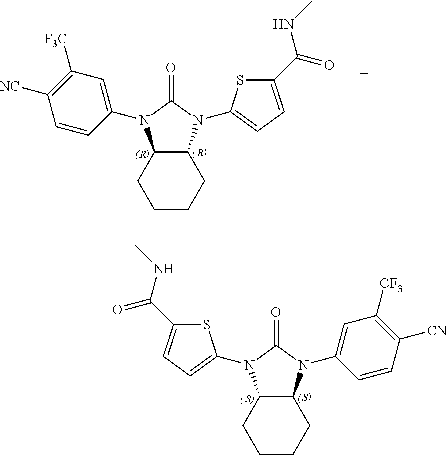

Trans-ethyl-5-(3-(4-cyano-3-(trifluoromethyl)phenyl)-2-oxooctahydro-1H-benzo[d]imidazol-1-yl)thiophene-2-carboxylate [racemic (±)] (0.110 g, 0.24 mmol) was treated with NaOH (0.0104 g, 0.26 mmol) as described for the synthesis of example 17 to give a residue. The residue was triturated with ether (5 mL×2) to give trans-5-{3-[4-cyano-3-(trifluoromethyl)phenyl]-2-oxo-octahydro-1H-1,3-benzodiazol-1-yl}thiophene-2-carboxylic acid [racemic (±)] (0.050 g, 0.11 mmol) as a white solid. The acid was treated with methylamine hydrochloride (0.018 g, 0.34 mmol) as described for the synthesis of example 18 to give a residue. The residue was purified by preparative TLC (hexanes/ethyl acetate=64) to give the title compound (0.020 g, 38.8%) as a white solid. HPLC: 98.53%; LCMS: m/z 449.1 [M+H]⁺; ¹H NMR (400 MHz, CDCl₃) δ 7.85 (d, 1H), 7.75 (d, 1H), 7.55 (dd, 1H), 7.36 (d, 1H), 6.79 (d, 1H), 5.89 (d, 1H), 3.78-3.73 (m, 1H), 3.62-3.56 (m, 1H), 2.99 (d, 3H), 2.59-2.56 (m, 1H), 2.39-2.35 (m, 1H), 2.08-2.03 (m, 2H), 1.70-1.50 (m, 4H).

### Example 41

### 4-((3aS,7 aS)-3-(4-cyano-3-(trifluoromethyl)phenyl)-2-oxooctahydro-1H-benzo[d]imidazol-1-yl)-2-fluoro-N-methylbenzothioamide

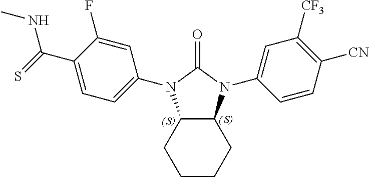

To a solution of example 1 b (Enantiopure) (0.08 g, 0.17 mmol) in xylene (5 mL) in a vial was added Lawesson's reagent (0.077 g, 0.19 mmol) and the vial was capped. After stirring for 2 h at 140° C., the reaction mixture was cooled to RT, transferred in a round bottom flask, and evaporated under reduced pressure to give a residue. The residue was purified by column chromatography on silica gel (dichloromethane/methanol=99:1) to give the title compound (0.040 g, 48.1%) as a yellow solid. HPLC: 95.94%; [α]_{D}²⁵=146 (c=0.03, MeOH); LCMS: m/z 477.1 [M+H]⁺; ¹H NMR (400 MHz, CDCl₃) δ 8.30 (t, 1H), 8.14 (bs, 1H), 7.85 (d, 1H), 7.75 (dd, 1H), 7.55 (dd, 1H), 7.15-7.11 (m, 1H), 7.05-7.02 (m, 1H), 3.75-3.50 (m, 2H), 3.39 (d, 3H), 2.42-2.37 (m, 2H) 2.07-2.05 (m, 2H), 1.59 (d, 2H), 1.51 (d, 2H).

### Example 42

### Trans-2-chloro-4-(3-(4-cyano-3-(trifluoromethyl)phenyl)-2-oxooctahydro-1H-benzo[d]imidazol-1-yl)-3-methylbenzonitrile (±)

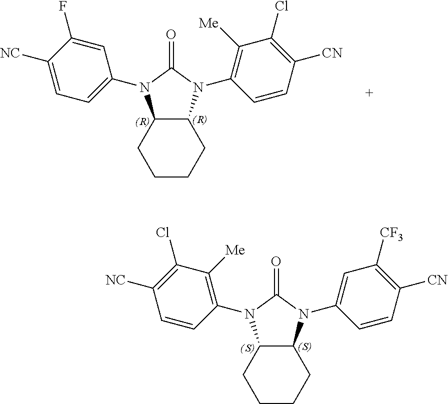

To a solution of trans-4-(2-oxooctahydro-1H-benzo[d]imidazol-1-yl)-2-(trifluoromethyl)benzonitrile [racemic (±)] (0.100 g, 0.32 mmol) in DMF (5 mL) was added sodium hydride (60% in mineral oil) (0.014 g, 0.36 mmol) at 0° C. and stirred for 30 min at the same temperature. 2-Chloro-4-fluoro-3-methylbenzonitrile (0.064 g, 0.36 mmol) in DMF (2 mL) was added dropwise at 0° C. and the reaction mixture was allowed to warm to RT. After stirring for 2 h, the reaction mixture was quenched with ammonium chloride solution (2 mL) and extracted with ethyl acetate (25 mL×2). The combined organic layer was washed with water (25 mL) followed by brine (25 mL), dried over Na₂SO₄, and concentrated under reduced pressure to give a residue. The residue was purified by column chromatography on silica gel (dichloromethane/methanol=1000 to 973) to give the title compound (0.040 g) as a white solid. The product obtained was repurified by preparative HPLC to give the title compound (0.020 g, 13.5%) as a white solid. HPLC: 95.52%; LCMS: m/z 459.1 [M+H]⁺; ¹H NMR (400 MHz, CDCl₃) δ 7.85 (d, 1H), 7.74 (d, 1H), 7.67 (d, 1H), 7.56 (dd, 1H), 7.17 (d, 1H), 3.86-3.81 (m, 1H), 3.75-3.70 (m, 1H), 2.44-2.38 (m, 2H), 2.39-2.34 (m, 3H), 2.07-1.99 (m, 2H), 1.64-1.47 (m, 4H).

COLUMN: Zorbax, Eclipse, C-18;

METHOD: Flow: 20.0 ml/min.; A:10 mM Ammonium acetate in Water; B: Acetonitrile.

| GRADIENT: | 1 | 2 | 3 |
|---|---|---|---|
| Time | 0.00 | 2.00 | 10.0 |
| % B | 50.0 | 60.0 | 90.0 |

### Example 43

### Trans-4-(3-(3,5-dichlorophenyl)-2-oxooctahydro-1H-benzo[d]imidazol-1-yl)-2-(trifluoromethyl)benzonitrile (±)

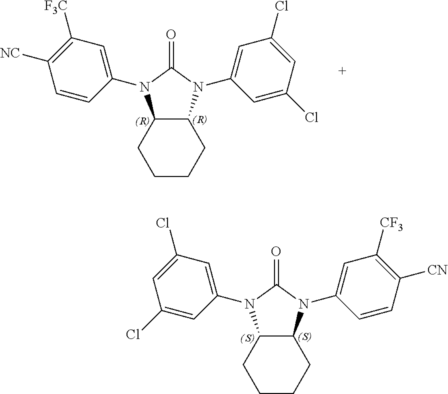

Trans-4-(2-oxooctahydro-1H-benzo[d]imidazol-1-yl)-2-(trifluoromethyl)benzonitrile [racemic (±)] (0.100 g, 0.32 mmol) was reacted with 1,3-dichloro-5-iodobenzene (0.088 g, 0.32 mmol) as described for the synthesis of example 1 to give a residue. The residue was purified by column chromatography using silica gel (dichloromethane/methanol=982) to give the title compound (0.040 g, 27.0%) as a white solid. HPLC: 95%; LCMS: m/z 454.4.1 [M+H]⁺; ¹H NMR (400 MHz, CDCl₃) δ 7.84 (d, 1H), 7.73 (s, 1H), 7.53 (dd, 1H), 7.23 (s, 1H), 7.16 (d, 2H), 3.75-3.62 (m, 2H), 2.35 (q, 2H), 2.05 (d, 2H), 1.57-1.48 (m, 4H).

### Example 44

### 4-((3aS,7aS)-3-(3,5-dichlorophenyl)-2-oxooctahydro-1H-benzo[d]imidazol-1-yl)-2-(trifluoromethyl)benzonitrile

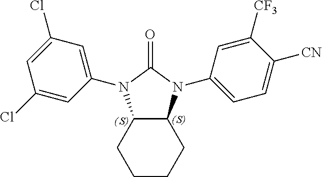

4-((3aS,7aS)-2-oxooctahydro-1H-benzo[d]imidazol-1-yl)-2-(trifluoromethyl)benzonitrile (Enantiopure) (0.309 g, 1.0 mmol) was reacted with 1,3-dichloro-5-iodobenzene (0.273 g, 1.0 mmol) as described for the synthesis of example 1 to give a residue. The residue was purified by column chromatography on silica gel (hexanes/ethyl acetate=46) to give the title compound (0.180 g, 40%) as a white crystals. LCMS: m/z 454 [M+H]⁺; ¹H NMR (400 MHz, CDCl₃) δ 7.84 (d, 1H), 7.74 (bs, 1H), 7.54 (dd, 1H), 7.23 (t, 1H), 7.18-7.14 (m, 2H), 3.73-3.62 (m, 2H), 2.40-2.31 (m, 2H), 2.05 (bs, 2H), 1.62-1.60 (m, 2H), 1.55-1.49 (m, 2H).

### Example 45

### 4-((3aS,7aS)-3-(4-acetyl-3-fluorophenyl)-2-oxooctahydro-1H-benzo[d]imidazol-1-yl)-2-(trifluoromethyl)benzonitrile

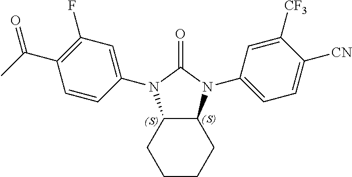

4-((3aS,7aS)-2-oxooctahydro-1H-benzo[d]imidazol-1-yl)-2-(trifluoromethyl)benzonitrile (Enantiopure)(0.250 g, 0.808 mmol) was treated with 1-(2-fluoro-4-iodophenyl)ethanone (0.234 g, 0.889 mmol) as described for the synthesis of example 1 to give a residue. The residue was purified by column chromatography on silica gel (hexanes/ethyl acetate=37) to give the title compound (0.110 g, 30%) as an off white solid. HPLC: 96.22%; LCMS: m/z 446 [M+H]⁺; ¹H NMR (400 MHz, CDCl₃) δ 7.95 (t, 1H), 7.86 (d, 1H), 7.74 (d, 1H), 7.55 (dd, 1H), 7.14 (dd, 1H), 7.09 (dd, 1H), 3.75 (ddd, 2H), 2.64 (d, 3H), 2.41 (t, 2H), 2.06 (d, 2H), 1.63 (m, 2H), 1.52 (m, 2H).

### Example 46

### 4-((3aS,7aS)-3-(3-fluoro-4-(hydroxymethyl)phenyl)-2-oxooctahydro-1H-benzo[d]imidazol-1-yl)-2-(trifluoromethyl)benzonitrile

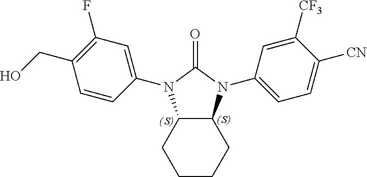

4-((3aS,7aS)-2-oxooctahydro-1H-benzo[d]imidazol-1-yl)-2-(trifluoromethyl)benzonitrile (Enantiopure) (0.100 g, 0.323 mmol) was treated with (2-fluoro-4-iodophenyl)methanol (0.089 g, 0.355 mmol) as described for the synthesis of example 1 to give a residue. The residue was purified by column chromatography on silica gel (dichloromethane/methanol=99.4/0.6) to give the title compound (0.034 g, 24%) as an off white solid. HPLC: 95.59%; LCMS: m/z 434 [M+H]⁺; ¹H NMR (400 MHz, DMSO-d₆) 8.19 (d, 1H), 7.95 (s, 1H), 7.76 (d, 1H), 7.48 (t, 1H), 7.15 (t, 2H), 5.24 (t, 1H), 4.53 (d, 2H), 3.96 (t, 1H), 3.81 (m, 1H), 2.19 (dd, 2H), 1.85 (d, 2H), 1.61 (t, 2H), 1.39 (m, 2H).

### Example 47

### Trans-4-(3-((6-chloropyridin-3-yl)methyl)-2-oxooctahydro-1H-benzo[d]imidazol-1-yl)-2-(trifluoromethyl)benzonitrile (±)

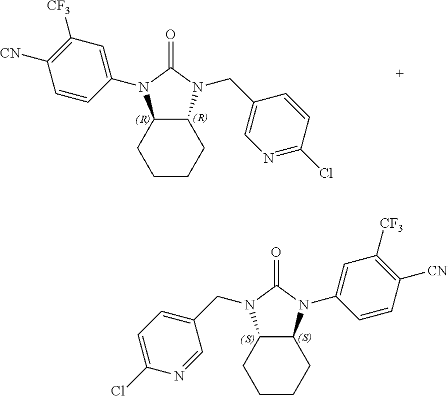

To a solution of trans-4-(2-oxooctahydro-1H-benzo[d]imidazol-1-yl)-2-(trifluoromethyl)benzonitrile [racemic (±)] (0.10 g, 0.32 mmol) in DMF (3 mL) was added NaH (60% in mineral oil) (0.014 g, 0.36 mmol) at 0° C. and stirred for 1 h. A solution of (6-chloropyridin-3-yl)methyl methanesulfonate (0.078 g, 0.36 mmol) in DMF (1 mL) was added dropwise and the resulting reaction mixture was allowed to warm to RT, and stirred for 16 h. The reaction mixture was quenched by adding satd NH₄Cl (1 mL) and extracted with EtOAc (15 mL×3). The combined organic layer was washed with water (15 mL×3), dried over Na₂SO₄, and concentrated under reduced pressure to give a residue. The residue was purified by preparative TLC (hexanes/ethyl acetate=11) to give the title compound (0.023 g, 16.0%) as a white solid. HPLC: 98%; LCMS: m/z 434.5 [M+H]⁺; ¹H NMR (400 MHz, CDCl₃) δ 8.35 (d, 1H), 7.81 (d, 1H), 7.73-7.69 (m, 2H), 7.55 (dd, 1H), 7.34 (d, 1H), 4.45 (q, 2H), 3.52 (ddd, 1H), 3.01 (ddd, 1H), 2.29 (dd, 1H), 2.05-2.03 (m, 1H), 1.95 (m, 2H), 1.57-1.34 (m, 4H).

### Example 48

### Trans-5-((-(4-cyano-3-(trifluoromethyl)phenyl)-2-oxooctahydro-1H-benzo[d]im-idazol-1-yl)methyl)picolinonitrile (±)

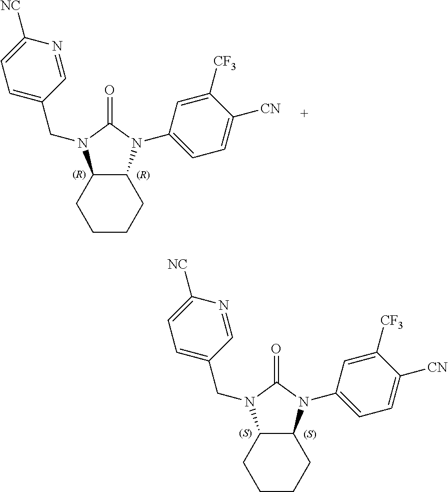

A solution of trans-4-(3-((6-chloropyridin-3-yl)methyl)-2-oxooctahydro-1H-benzo[d]imidazol-1-yl)-2-(trifluoromethyl)benzonitrile [racemic (±)] (0.100 g, 0.230 mmol) in dry DMA (2.0 mL) was degassed with Argon for 10 min. Zn (0.015 g, 0.230 mmol), Pd(dba)₃ (0.0105 g, 0.011 mmol) and dppf (1 mg) were added and degassing continued for another 10 min. Zn(CN)₂ (0.030 g, 0.253 mmol) was added and the resulting reaction mixture was heated to 110° C. for 12 h. The reaction mixture was cooled to RT, diluted with water (20 mL), and extracted with DCM (20 mL×3). The combined organic layer was washed with brine (20 mL×3), dried over Na₂SO₄, and concentrated under reduced pressure to give a residue. The residue was purified by column chromatography on silica gel (hexanes/ethyl acetate=11) to give the title compound (0.040 g, 40.8%) as a white solid. HPLC=96.11%; LCMS: m/z 426.4 [M+H]⁺; ¹H NMR (400 MHz, CDCl₃) δ 8.75 (s, 1H), 7.80-7.90 (m, 2H), 7.75-7.65 (m, 2H), 7.50 (d, 1H), 4.70 (d, 1H), 4.40 (d, 1H), 3.60 (t, 1H), 3.10 (t, 1H), 2.30 (d, 3H), 2.0 (d, 1H), 1.58-1.45 (m, 4H).

### Example 49

### Trans-4-(34(6-(1H-imidazol-1-yl)pyridin-3-yl)methyl)-2-oxooctahydro-1H-benzo[d]imidazol-1-yl)-2-(trifluoromethyl)benzonitrile (±)

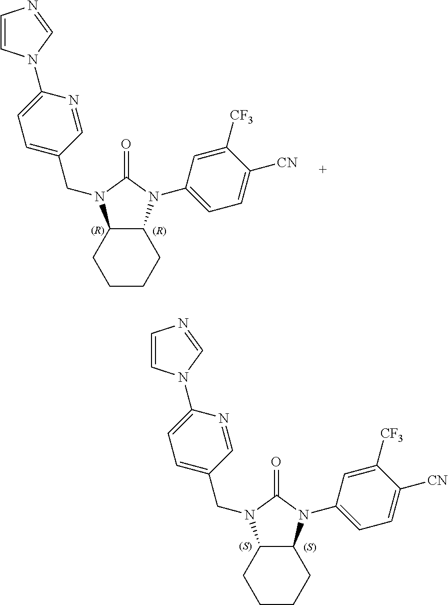

To a degassed solution of trans-4-(34(6-chloropyridin-3-yl)methyl)-2-oxooctahydro-1H-benzo[d]imidazol-1-yl)-2-(trifluoromethyl)benzonitrile [racemic (±)] (0.200 g, 0.461 mmol) and imidazole (0.063 g, 0.922 mmol) in dry DMF (5.0 mL) was added Cs₂CO₃ (0.450 g, 1.382 mmol) followed by Cu₂O (0.066 g, 0.461 mmol) at RT. The resulting reaction mixture was stirred under N₂ atmosphere at 90° C. for 12 h. The reaction mixture was cooled to RT, diluted with water (20 mL), and extracted with ethyl acetate (20 mL×3). The combined organic layer was washed with brine (20 mL×3), dried over Na₂SO₄, and concentrated under reduced pressure to give a residue. The residue was purified by column chromatography on silica gel (hexanes/ethyl acetate=82) to give the title compound (0.025 g, 11.6%) as a white solid. HPLC=97.95%; LCMS: m/z 467.3 [M+H]⁺; ¹H NMR (400 MHz, CDCl₃) δ 8.70 (bs, 1H), 8.60 (s, 1H), 8.20 (d, 1H), 8.05-7.98 (m, 3H), 7.90 (d, 1H), 7.70 (d, 1H), 7.30 (bs, 1H), 4.60-4.40 (m, 2H), 3.80 (t, 1H), 3.20 (t, 1H), 2.30 (d, 1H), 2.20 (d, 1H), 1.80 (t, 2H), 1.60 (m, 1H), 1.43-1.35 (m, 3H).

### Example 50

### Trans-4-(3-((6-(1H-pyrazol-1-yl)pyridin-3-yl)methyl)-2-oxooctahydro-1H-benzo[d]imidazol-1-yl)-2-(trifluoromethyl)benzonitrile (±)

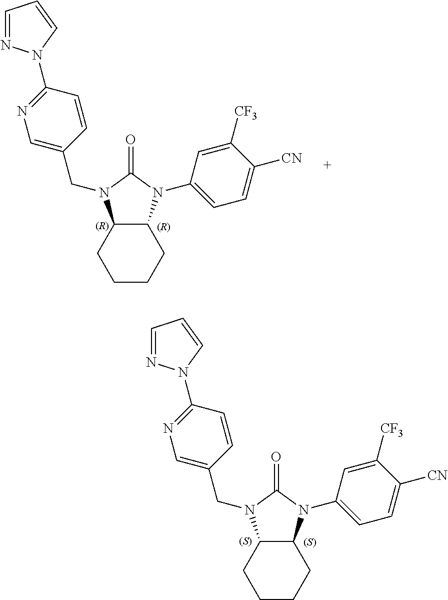

Trans-4-(3-((6-chloropyridin-3-yl)methyl)-2-oxooctahydro-1H-benzo[d]imidazol-1-yl)-2-(trifluoromethyl)benzonitrile [racemic (±)] (0.100 g, 0.230 mmol) was reacted with pyrazole (0.031 g, 0.461 mmol) as described for the synthesis of example 49 to give a residue. The residue was purified by column chromatography on silica gel (hexanes/ethyl acetate=73) to give the title compound (0.025 g, 11.6%) as a white solid. HPLC=98.12%; LCMS: m/z 467.4 [M+H]⁺; ¹H NMR (400 MHz, CDCl₃) δ 8.60 (s, 1H), 8.40 (s, 1H), 8.20 (d, 1H), 8.10-8.00 (m, 3H), 7.80 (s, 1H), 7.70 (d, 1H), 6.60 (s, 1H), 4.50 (m, 2H), 3.70 (t, 1H), 3.1 (t, 1H), 2.20 (d, 1H), 2.10 (s, 1H), 1.70 (d, 2H), 1.5 (d, 1H), 1.45-1.35 (m, 3H).

### Examples 51 and 52

### Trans-4-(3-((1H-pyrazol-3-yl)methyl)-2-oxooctahydro-1H-benzo[d]imidazol-1-yl)-2-(trifluoromethyl)benzonitrile (±) and trans-4-(3-(3-methyl-1H-pyrazol-4-yl)-2-oxooctahydro-1H-benzo[d]imidazol-1-yl)-2-(trifluoromethyl)benzonitrile (±)

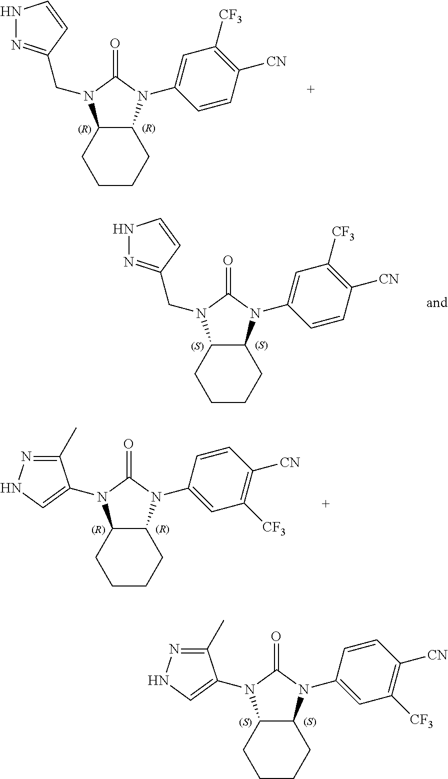

To a trans-4-(2-oxooctahydro-1H-benzo[d]imidazol-1-yl)-2-(trifluoromethyl)benzonitrile [racemic (±)] (1.00 g, 3.247 mmol) in DMF (10 mL) was added Cs₂CO₃ (5.29 g, 16.236 mmol) at RT. After stirring for 15 min., chloroacetone (2.10 g, 22.698 mmol) was added, and the reaction mixture was heated to 120° C. for 10 h. The reaction mixture was cooled to RT, diluted with ethyl acetate (250 mL), and washed with water (50 mL×2). The organic layer was dried over Na₂SO₄ and concentrated under reduced pressure to give a residue. The residue was purified by column chromatography on silica gel (hexanes/ethyl acetate=64) to give trans-4-(2-oxo-3-(2-oxopropyl)octahydro-1H-benzo[d]imidazol-1-yl)-2-(trifluoromethyl)benzonitrile [racemic (±)] (0.550 g, 84.0%) as a brown solid. LCMS: m/z 366 [M+H]⁺. DMF.DMA (5 mL) was added to trans-4-(2-oxo-3-(2-oxopropyl)octahydro-1H-benzo[d]imidazol-1-yl)-2-(trifluoromethyl)benzonitrile (±) (0.550 g, 1.507 mmol) and the resulting reaction mixture was heated to 100° C. for 15 h. The reaction mixture was cooled to RT, diluted with CH₂Cl₂ (100 mL), and washed with water (50 mL×2). The organic layer was separated, dried over Na₂SO₄ and concentrated under reduced pressure to give a mixture of trans-4-(3-(1-(dimethylamino)-3-oxobut-1-en-2-yl)-2-oxooctahydro-1H-benzo[d]imidazol-1-yl)-2-(trifluoromethyl)benzonitrile and trans-4-(3-(4-(dimethylamino)-2-oxobut-3-en-1-yl)-2-oxooctahydro-1H-benzo[d]imidazol-1-yl)-2-(trifluoromethyl)benzonitrile (0.530 g, 83.0%) as a red liquid. LCMS: m/z 421[M+H]⁺. The above mixture (0.530 g, 1.262 mmol) was dissolved in EtOH (6 mL) and treated with hydrazine hydrate (0.126 g, 2.52 mmol). The resulting reaction mixture was heated to 120° C. for 30 min, cooled to RT, and concentrated under reduced pressure to give a residue. The residue was purified by column chromatography on silica gel (chloroform/methanol=991) to give trans-4-(3-((1H-pyrazol-3-yl)methyl)-2-oxooctahydro-1H-benzo[d]imidazol-1-yl)-2-(trifluoromethyl)benzonitrile[racemic (±)] (0.080 g, 16.0%) as a yellow solid. HPLC: 94.67%; LCMS: m/z 390.2 [M+H]⁺; ¹H NMR (400 MHz, CDCl₃) δ 7.81 (d, 1H), 7.74 (s, 1H), 7.54 (d, 2H), 6.30 (s, 1H), 4.65 (d, 1H), 4.31 (d, 1H), 3.49 (t, 1H), 2.99 (t, 1H), 2.19 (t, 2H), 1.92 (t, 2H), 1.31 (m, 4H). Further elution on silica gel (chloroform/methanol=982) gave trans-4-(3-(3-methyl-1H-pyrazol-4-yl)-2-oxooctahydro-1H-benzo[d]imidazol-1-yl)-2-(trifluoro-methyl)benzonitrile [racemic (±)] (0.015 g, 3.0%) as an off white solid. HPLC: 95.34%; LCMS: m/z 390.1 [M+H]⁺; ¹H NMR (400 MHz, CDCl₃) δ 7.81 (d, 1H), 7.76 (s, 1H), 7.58 (d, 1H), 7.46 (s, 1H), 3.66 (t, 1H), 3.42 (t. 1H), 2.35 (d, 1H), 2.26 (s, 3H), 1.99 (d, 3H), 1.52 (m, 4H).

### Example 53

### Trans-5-(3-(4-cyano-3-(trifluoromethyl)phenyl)-2-oxooctahydro-1H-benzo[d]imidazol-1-yl)-N-methylpicolinamide (±)

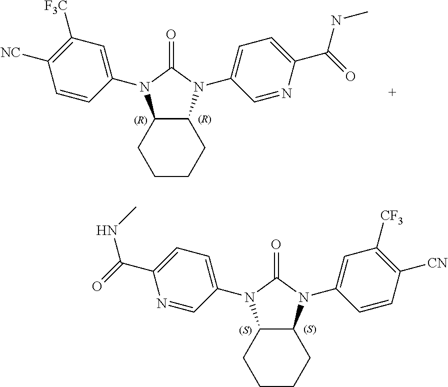

Trans-4-(2-oxooctahydro-1H-benzo[d]imidazol-1-yl)-2-(trifluoromethyl)benzonitrile [racemic (±)] (0.100 g, 0.32 mmol) was reacted with 5-bromo-N-methylpicolinamide (0.07 g, 0.32 mmol) as described for the synthesis of example 1 to give a residue. The residue was purified by preparative HPLC to give the title compound (0.025 g, 17.4%) as a white solid. HPLC: 95%; LCMS: m/z 443.8 [M+H]⁺; ¹H NMR (400 MHz, CDCl₃) δ 8.53 (s, 1H), 8.26 (d, 1H), 7.92 (bs, 1H), 7.87 (d, 1H), 7.75 (s, 1H), 7.70 (dd, 1H), 7.57 (d, 1H), 3.80-3.78 (m, 2H), 3.05 (d, 3H), 2.40 (t, 2H), 2.08 (d, 2H), 1.63-1.6 (m, 4H).

COLUMN: Waters X Bridge C-18.

METHOD: Flow: 20.0 mL/min; Mobile Phase: A: 10 mM NH₄OAc in Water; B: Acetonitrile.

| GRADIENT: | 1 | 2 | 3 |
|---|---|---|---|
| Time | 0.00 | 2.00 | 8.00 |
| % B | 30.0 | 40.0 | 80.0 |

### Example 54

### 5-((3aS,7aS)-3-(4-cyano-3-(trifluoromethyl)phenyl)-2-oxooctahydro-1H-benzo[d]imidazol-1-yl)-N-methylpicolinamide

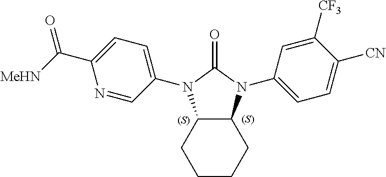

4-((3aS,7aS)-2-oxooctahydro-1H-benzo[d]imidazol-1-yl)-2-(trifluoromethyl)benzonitrile (Enantiopure) (0.370 g, 1.2 mmol) was reacted with 5-bromo-N-methylpicolinamide (0.300 g, 1.4 mmol) as described for the synthesis of example 1 to give a residue. The residue was purified by preparative HPLC to give the title compound (0.068 g, 13.0%) as a white solid. LCMS: m/z 444 [M+H]⁺; ¹H NMR (400 MHz, CDCl₃) δ 8.52 (d, 1H), 8.24 (d, 1H), 7.93 (bs, 1H), 7.85 (d, 1H), 7.74 (bs, 1H), 7.69 (dd, 1H), 7.56 (dd, 1H), 3.79-3.77 (m, 2H), 3.04 (d, 3H), 2.42-2.32 (m, 2H), 2.1-2.04 (m, 2H), 1.70-1.45 (m, 4H).

COLUMN: Zorbax C-18 XDB

METHOD: FLOW: 20.0 mL/min

A: 0.1% TFA in Water

B: ACETONITRILE

| GRADIENT | Time | % B |
|---|---|---|
| 1 | 0.00 | 50.0 |
| 2 | 2.00 | 60.0 |
| 3 | 10.0 | 80.0 |

### Example 55

### 5-((3aS,7aS)-3-(4-cyano-3-(trifluoromethyl)phenyl)-2-oxooctahydro-1H-benzo[d]imidazol-1-yl)picolinamide

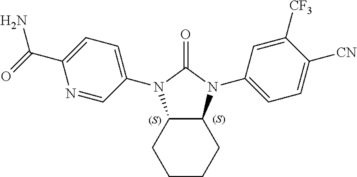

4-((3aS,7aS)-2-oxooctahydro-1H-benzo[d]imidazol-1-yl)-2-(trifluoromethyl)benzonitrile (Enantiopure) (0.450 g, 1.456 mmol) was reacted with N,N-dibenzyl-5-bromopicolinamide (0.550 g, 1.456 mmol) as described for the synthesis of example 1 to give a residue (0.450 g). The residue (0.250 g, 0.410 mmol) was dissolved in DCM (10 mL) and treated with conc. H₂SO₄ (2 mL). After stirring for 1 h at RT, the reaction mixture was neutralized with 6N NaOH solution and extracted with ethyl acetate (30 mL×3). The combined organic layer was washed with water (50 mL) followed by brine (50 mL), dried over Na₂SO₄ and concentrated under reduced pressure to give a residue. The residue was purified by column chromatography on silica gel (dichloromethane/methanol=982) to give the title compound (0.012 g, 6.8%) as a white solid. LCMS: m/z 430.1[M+H]⁺; ¹H NMR (400 MHz, CDCl₃) δ 8.56 (d, 1H), 8.26 (d, 1H), 7.86 (d, 1H), 7.74-7.71 (m, 3H), 7.58-7.55 (m, 1H), 5.55 (bs, 1H), 3.80 (d, 2H), 2.39 (t, 2H), 2.07 (d, 2H), 1.63-1.56 (m, 4H).

### Example 56

### 4-((3aS,7aS)-3-(4-cyano-3-(trifluoromethyl)phenyl)-2-oxooctahydro-1H-benzo[d]imidazol-1-yl)-N-methylpicolinamide

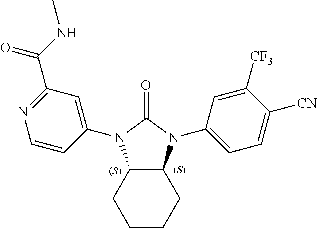

4-((3aS,7aS)-2-oxooctahydro-1H-benzo[d]imidazol-1-yl)-2-(trifluoromethyl)benzonitrile (Enantiopure) (0.070 g, 0.226 mmol) was reacted with 4-bromo-N-methylpicolinamide (0.058 g, 0.271 mmol) as described for the synthesis of example 1 to give a residue. The residue was purified by preparative TLC (dichloromethane/methanol=982) to give the title compound (0.020 g, 20%) as a white solid. HPLC: 98.69%; LCMS: m/z 444.1 [M+H]⁺; ¹H NMR (400 MHz, DMSO-d6) δ 8.80 (d, 1H), 8.59 (d, 1H), 8.22 (d, 1H), 7.99 (bd, 1H), 7.79 (d, 1H), 7.55 (dd, 1H), 4.03 (t, 2H), 2.82 (d, 3H), 2.36-2.26 (dd, 2H), 1.88 (bs, 2H), 1.64-1.60 (m, 2H), 1.44-1.40 (m, 2H).

### Example 57

### 4-((3aS,7aS)-3-(4-cyano-3-(trifluoromethyl)phenyl)-2-oxooctahydro-1H-benzo[d]imidazol-1-yl)picolinamide

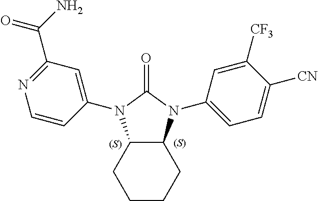

4-((3aS,7aS)-2-oxooctahydro-1H-benzo[d]imidazol-1-yl)-2-(trifluoromethyl)benzonitrile (Enantiopure) (0.200 g, 0.645 mmol) was reacted with N,N-dibenzyl-4-bromopicolinamide (0.295 g, 0.775 mmol) as described for the synthesis of example 1 to give a residue. The residue was purified by column chromatography on silica gel (dichloromethane/methanol=991) to give N,N-dibenzyl-4-((3aS,7aS)-3-(4-cyano-3-(trifluoromethyl)phenyl)-2-oxooctahydro-1H-benzo[d]imidazol-1-yl)picolinamide (0.220 g, 55%) as a white solid. LCMS: m/z 610.0 [M+H]⁺; ¹H NMR (300 MHz, CDCl₃) δ 8.56 (d, 1H), 7.87 (d, 1H), 7.72 (bs, 1H), 7.54-7.52 (m, 2H), 7.40-7.26 (m, 8H), 4.69 (bs, 4H), 3.77 (d, 2H), 2.48-2.36 (m, 2H), 2.10-2.05 (m, 2H), 1.64-1.50 (m, 7H). To a solution of N,N-dibenzyl-4-((3aS,7aS)-3-(4-cyano-3-(trifluoromethyl)phenyl)-2-oxooctahydro-1H-benzo[d]imidazol-1-yl)picolinamide (Enantiopure) (0.220 g, 0.360 mmol) in DCM (1 mL) was added conc. H₂SO₄ (0.5 mL) at RT and the reaction mixture was stirred for 4 h. The reaction mixture was cooled to 0° C., quenched with satd. bicarbonate solution (10 mL), and extracted with DCM (20 mL×2). The combined organic layer was washed with brine solution (20 mL), dried over Na₂SO₄ and concentrated under reduced pressure to give a residue. The residue was purified by column chromatography on silica gel (dichloromethane/methanol=946) to give the title compound (0.020 g, 12%) as a white solid. HPLC: 98.91%; LCMS: m/z 430[M+H]⁺; ¹H NMR (400 MHz, CDCl₃) δ 8.56 (d, 1H), 7.92-7.860 (m, 3H), 7.74 (bs, 1H), 7.64 (dd, 1H), 7.54 (dd, 1H), 5.60 (bs, 1H), 3.92-3.75 (m, 2H), 2.30 (d, 1H), 2.20 (d, 1H), 2.01-2.06 (m, 2H), 1.42-1.72 (m, 4H).

### Example 58

### 4-((3aS,7aS)-3-(2,3-dihydrobenzofuran-5-yl)-2-oxooctahydro-1H-benzo[d]imidazol-1-yl)-2-(trifluoromethyl)benzonitrile

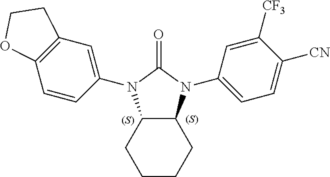

4-((3aS,7aS)-2-oxooctahydro-1H-benzo[d]imidazol-1-yl)-2-(trifluoromethyl)benzonitrile (Enantiopure) (0.093 g, 0.3 mmol) was reacted with 5-bromo-2,3-dihydrobenzofuran (0.059 g, 0.3 mmol) as described for the synthesis of example 1 to give a residue. The residue was purified by column chromatography on silica gel (hexanes/ethyl acetate=37) to give the title compound (0.021 g, 16.4%) as a yellow solid. LCMS: m/z 428 [M+H]⁺; ¹H NMR (400 MHz, CDCl₃) δ 7.82-7.79 (m, 2H), 7.56 (d, 1H), 7.11 (bs, 1H), 6.93 (d, 1H), 6.80 (d, 1H), 4.60 (t, 2H), 3.67 (t, 1H), 3.54 (t, 1H), 3.23 (t, 2H), 2.38 (d, 1H), 2.14 (d, 1H), 2.30-1.95 (m, 2H), 1.70-1.45 (m, 4H).

### Example 59

### Trans-4-(3-(2-fluoropyridin-3-yl)-2-oxooctahydro-1H-benzo[d]imidazol-1-yl)-2-(trifluoromethyl)benzonitrile (±)

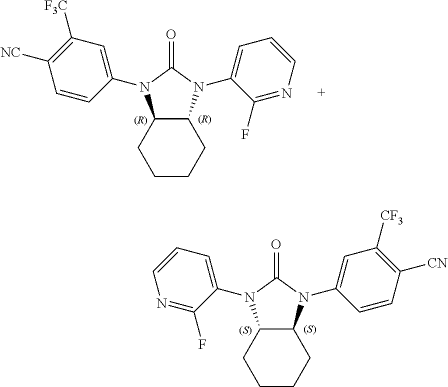

Trans-4-(2-oxooctahydro-1H-benzo[d]imidazol-1-yl)-2-(trifluoromethyl)benzonitrile [racemic (±)] (0.100 g, 0.32 mmol) was reacted with 2-fluoro-3-iodopyridine (0.07 g, 0.32 mmol) as described for the synthesis of example 1 to give a residue. The residue was purified by preparative TLC (hexanes/ethyl acetate=46) to give the title compound (0.022 g, 16.8%) as a white solid. HPLC: 90.94%; LCMS: m/z 405.4 [M+H]⁺; ¹H NMR (400 MHz, CDCl₃) δ 8.15 (d, 1H), 7.85 (d, 1H), 7.75 (m, 2H), 7.60-7.57 (m, 1H), 7.30-7.26 (m, 1H), 3.77-3.75 (m, 2H), 2.39 (d, 1H), 2.05 (m, 3H), 1.57-1.50 (m, 4H).

### Example 60

### Trans-4-(3-(4-chlorophenyl)-2-oxooctahydro-1H-benzo[d]imidazol-1-yl)-2-(trifluoromethyl)benzonitrile (±)

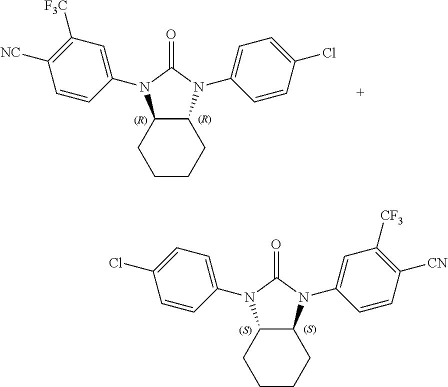

Trans-4-(2-oxooctahydro-1H-benzo[d]imidazol-1-yl)-2-(trifluoromethyl)benzonitrile [racemic (±)] (0.100 g, 0.32 mmol) was reacted with 1-chloro-4-iodobenzene (0.08 g, 0.32 mmol) as described for the synthesis of example 1 to give a residue. The residue was purified by preparative TLC (hexanes/ethyl acetate=11) to give the title compound (0.015 g, 11.0%) as a white solid. HPLC: 96.52%; LCMS: m/z 420.5 [M+H]⁺; ¹H NMR (400 MHz, CDCl₃) δ 7.84 (d, 1H), 7.77 (d, 1H), 7.55 (dd, 1H), 7.40-7.37 (m, 2H), 7.22-7.19 (m, 2H), 3.73-3.65 (m, 2H), 2.40 (d, 1H), 2.27 (d, 1H), 2.06-2.01 (m, 2H), 1.61-1.49 (m, 4H).

### Example 61

### 4-((3aS,7aS)-3-(4-chlorophenyl)-2-oxooctahydro-1H-benzo[d]imidazol-1-yl)-2-(trifluoromethyl)benzonitrile

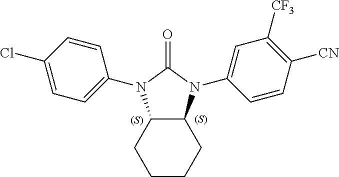

4-((3aS,7aS)-2-oxooctahydro-1H-benzo[d]imidazol-1-yl)-2-(trifluoromethyl)benzonitrile (Enantiopure) (0.200 g, 0.65 mmol) was reacted with 1-chloro-4-iodobenzene (0.15 g, 0.65 mmol) as described for the synthesis of example 1 to give a residue. The residue was purified by column chromatography on silica gel (dichloromethane/methanol=1000 to 99.5/0.5) to give the title compound (0.047 g, 17.3%) as a white solid. HPLC: 96.23%; LCMS: m/z 420.4 [M+H]⁺; ¹H NMR (400 MHz, CDCl₃) δ 7.83 (d, 1H), 7.76 (s, 1H), 7.56 (dd, 1H), 7.39 (d, 2H), 7.19 (d, 2H), 3.75-3.62 (m, 2H), 2.4 (d, 2H), 2.25 (d, 2H), 2.03 (m, 2H), 1.56-1.52 (m, 2H).

### Example 62

### 4-((3aS,7aS)-3-(4-cyclopropylpyridin-3-yl)-2-oxooctahydro-1H-benzo[d]imidazol-1-yl)-2-(trifluoromethyl)benzonitrile

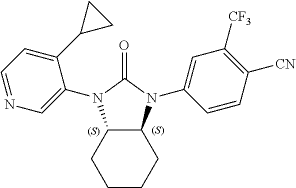

4-((3aS,7aS)-2-oxooctahydro-1H-benzo[d]imidazol-1-yl)-2-(trifluoromethyl)benzonitrile (Enantiopure) (0.20 g, 0.65 mmol) was reacted with 4-cyclopropyl-3-iodopyridine (0.16 g, 0.65 mmol) as described for the synthesis of example 1 to give a residue. The residue was purified by preparative TLC (hexanes/ethyl acetate=37) to give the title compound (0.017 g, 6.2%) as a white solid. HPLC: 90.2%; LCMS: m/z 426.8 [M+H]⁺; ¹H NMR (400 MHz, CDCl₃) δ 8.49-8.42 (m, 2H), 7.86-7.84 (m, 1H), 7.76 (d, 1H), 7.63-7.58 (m, 1H), 7.57-7.55 (m, 1H), 3.83-3.67 (m, 2H), 2.43-2.32 (m, 2H), 2.04-1.97 (m, 4H) 1.60-1.22 (m, 2H), 1.16-1.00 (m, 1H), 0.92-0.89 (m, 2H), 0.70-0.65 (m, 2H).

### Example 63

### 4-((3aS,7aS)-3-(4-methylpyridin-3-yl)-2-oxooctahydro-1H-benzo[d]imidazol-1-yl)-2-(trifluoromethyl)benzonitrile

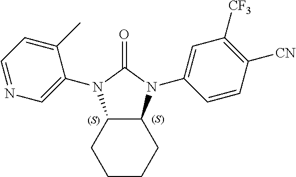

4-((3aS,7aS)-2-oxooctahydro-1H-benzo[d]imidazol-1-yl)-2-(trifluoromethyl)benzonitrile (Enantiopure) (0.20 g, 0.65 mmol) was reacted with 3-bromo-4-methylpyridine (0.11 g, 0.65 mmol) as described for the synthesis of example 1 to give a residue. The residue was purified by preparative TLC (hexanes/ethyl acetate=37) to give the title compound (0.02 g, 7.7%) as a white solid. HPLC: 96.9%; LCMS: m/z 401.6 [M+H]⁺; ¹H NMR (400 MHz, CDCl₃) δ 8.48-8.42 (m, 2H), 7.84 (d, 1H), 7.76 (s, 1H), 7.61-7.58 (m, 1H), 7.25-7.24 (m, 1H), 3.81-3.74 (m, 2H), 2.40 (d, 2H), 2.29 (s, 3H), 2.03-1.96 (m, 4H), 1.60-1.45 (m, 2H).

### Example 64

### Ethyl 4-((3aS,7aS)-3-(4-cyano-3-(trifluoromethyl)phenyl)-2-oxooctahydro-1H-benzo[d]imidazol-1-yl)benzoate

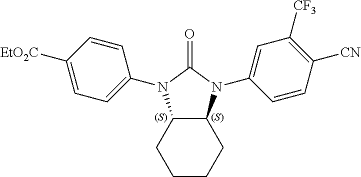

4-((3aS,7aS)-2-oxooctahydro-1H-benzo[d]imidazol-1-yl)-2-(trifluoromethyl)benzonitrile (Enantiopure) (0.30 g, 0.97 mmol) was reacted with ethyl 4-bromobenzoate (0.22 g, 0.97 mmol) as described for the synthesis of example 1 to give a residue. The residue was purified by column chromatography on silica gel (dichloromethane/methanol=1000 to 991) to give the title compound (0.210 g, 47.3%) as a white solid. LCMS: m/z 458.5 [M+H]⁺.

### Example 65

### 4-((3aS,7aS)-3-(4-cyano-3-(trifluoromethyl)phenyl)-2-oxooctahydro-1H-benzo[d]imidazol-1-yl)benzoic acid

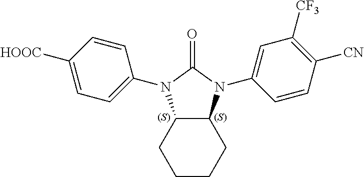

Ethyl 4-((3aS,7aS)-3-(4-cyano-3-(trifluoromethyl)phenyl)-2-oxooctahydro-1H-benzo[d]imidazol-1-yl)benzoate (Enantiopure) (0.20 g, 0.47 mmol) was reacted with sodium hydroxide (0.02 g, 0.48 mmol) as described for the synthesis of example 17 to the title compound (0.052 g, 27.5%) as an off white solid. The crude product was used as such for the next step without further purification.

### Example 66

### 4-((3aS,7aS)-3-(4-cyano-3-(trifluoromethyl)phenyl)-2-oxooctahydro-1H-benzo[d]imidazol-1-yl)benzamide

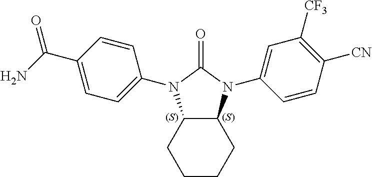

4-((3aS,7aS)-3-(4-cyano-3-(trifluoromethyl)phenyl)-2-oxooctahydro-1H-benzo[d]imidazol-1-yl)benzoic acid (Enantiopure) (0.050 g, 0.12 mmol) was reacted with ammonium chloride (0.020 g, 0.35 mmol) as described for the synthesis of example 18 to give a residue. The residue was purified by preparative TLC (hexanes/ethyl acetate=11) to give title compound (0.011 g, 22%) as a white solid. HPLC: 91.63%; LCMS: m/z 428.8 [M+H]⁺; ¹H NMR (400 MHz, CDCl₃) δ 7.88 (t, 3H), 7.76 (d, 1H), 7.55 (dd, 1H), 7.39 (d, 2H), 3.76 (m, 2H), 2.42-2.36 (m, 2H), 2.05 (m, 2H), 1.59-1.53 (m, 4H).

### Intermediate 4: N,N-dibenzyl-4-bromo-2-fluorobenzenesulfonamide

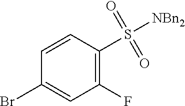

To a solution of 4-bromo-2-fluorobenzene-1-sulfonyl chloride (0.30 g, 1.1 mmol) and triethylamine (0.31 mL, 2.20 mmol) in DCM (10 mL) was added dibenzylamine (0.24 g, 1.21 mmol) at 0° C. The resulting reaction mixture was stirred for 4 h at RT. The reaction mixture was diluted with water (10 mL) and extracted with DCM (10 mL×3). The combined organic layer was washed with satd NaHCO₃ solution (10 mL×2) followed by water (10 mL) and brine (10 mL), dried over Na₂SO₄, and concentrated under reduced pressure to give a residue. The residue was triturated with diethyl ether to give the title compound (0.40 g, 84%) as an off white solid. LCMS: m/z 433.1 [M−H]⁺.

### Intermediate 5: Trans-N,N-dibenzyl-4-(3-(4-cyano-3-(trifluoromethyl)phenyl)-2-oxooctahydro-1H-benzo[d]imidazol-1-yl)-2-fluorobenzenesulfonamide (±)

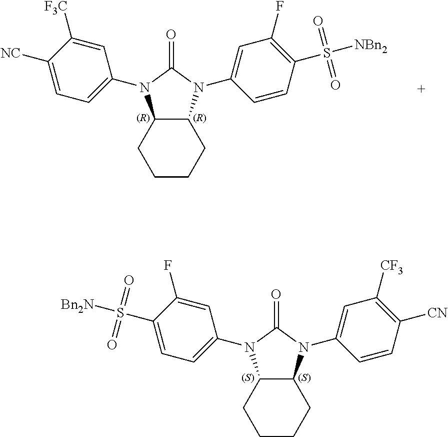

Trans-4-(2-oxooctahydro-1H-benzo[d]imidazol-1-yl)-2-(trifluoromethyl)-benzonitrile [racemic (±)] (0.20 g, 0.65 mmol) was reacted with N,N-dibenzyl-4-bromo-2-fluorobenzenesulfonamide (0.28 g, 0.65 mmol) as described for the synthesis of example 1 to give a residue. The residue was purified by column chromatography on silica gel (dichloromethane/methanol=1000 to 991) to give the title compound (0.156 g, 36.4%) as a white solid. The crude product was used as such for the next step without further purification.

### Example 67

### Trans-4-(3-(4-cyano-3-(trifluoromethyl)phenyl)-2-oxooctahydro-1H-benzo[d]imidazol-1-yl)-2-fluorobenzenesulfonamide (±)

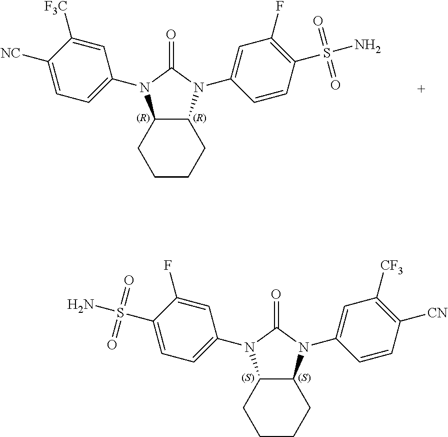

To a solution of trans-N,N-dibenzyl-4-(3-(4-cyano-3-(trifluoromethyl)phenyl)-2-oxooctahydro-1H-benzo[d]imidazol-1-yl)-2-fluorobenzenesulfonamide [racemic (±)] (0.150 g, 0.23 mmol) in dichloromethane (5 mL) was added conc. H₂SO₄ (1 mL) at 0° C. and the resulting reaction mixture was allowed to warm to RT. After stirring for 30 min, ice-water (10 mL) was added and extracted with dichloromethane (25 mL×3). The combined organic layer was washed with saturated NaHCO₃ solution (20 mL×3) followed by water (20 mL), dried over Na₂SO₄, and concentrated under reduced pressure to give a residue. The residue was purified by preparative TLC (hexanes/ethyl acetate=64) to give the title compound (0.045 g, 41.2%) as a white solid. HPLC: 94.42%; LCMS: m/z 483.1 [M+H]⁺; ¹H NMR (400 MHz, CDCl₃) δ 7.92-7.85 (m, 2H), 7.74 (d, 1H), 7.54 (dd, 1H), 7.23 (d, 1H), 7.11 (dd, 1H), 5.08 (s, 2H), 3.75 (ddd, 2H), 2.41 (ddd, 2H), 2.07 (d, 2H), 1.63-1.52 (m, 4H).

### Example 68

### Ethyl 4-((3aS,7aS)-3-(4-cyano-3-(trifluoromethyl)phenyl)-2-oxooctahydro-1H-benzo[d]imidazol-1-yl)-2-methylbenzoate

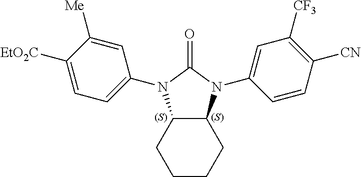

4-((3aS,7aS)-2-oxooctahydro-1H-benzo[d]imidazol-1-yl)-2-(trifluoromethyl)benzonitrile (0.30 g, 0.97 mmol) was reacted with ethyl 4-bromo-2-methylbenzoate (0.24 g, 0.97 mmol) as described for the synthesis of example 1 to give a residue. The residue was purified by column chromatography on silica gel (dichloromethane/methanol=1000 to 991) to give the title compound (0.20 g, 43.7%) as a white solid. The crude product was used as such for the next step without further purification.

### Example 69

### 4-((3aS,7aS)-3-(4-cyano-3-(trifluoromethyl)phenyl)-2-oxooctahydro-1H-benzo[d]imidazol-1-yl)-2-methylbenzoic acid

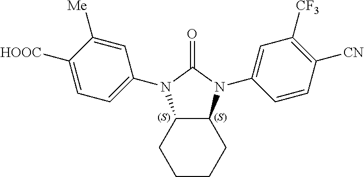

To a solution of ethyl 4-((3aS,7aS)-3-(4-cyano-3-(trifluoromethyl)phenyl)-2-oxooctahydro-1H-benzo[d]imidazol-1-yl)-2-methylbenzoate (Enantiopure) (0.2 g, 0.45 mmol) in THF (10 mL) was added aqueous solution of lithium hydroxide monohydrate (0.010 g, 0.50 mmol) at RT and the resulting reaction mixture was stirred for 12 h. The reaction mixture was concentrated under reduced pressure to give a residue. The residue was dissolved in water (25 mL) and extracted with diethyl ether (25 mL×2). The pH of aqueous layer was adjusted to 2 using conc. HCl and extracted with ethyl acetate (50 mL×2). The combined organic layer was washed with water (50 mL) followed by brine (50 mL), dried over Na₂SO₄, and concentrated under reduced pressure to give a residue. The residue was triturated with ether (5 mL×2) to give the title compound (0.060 g, 31.9%) as an off white solid. HPLC: 92.96%; LCMS: m/z 444.2 [M+H]⁺; ¹H NMR (400 MHz, CDCl₃) δ 8.13 (d, 1H), 7.85 (d, 1H), 7.77 (s, 1H), 7.55 (dd, 1H), 7.21 (s, 1H), 7.15 (dd, 1H), 3.75 (m, 2H), 2.68 (s, 3H), 2.45 (m, 2H), 2.55 (m, 2H), 1.65-1.50 (m, 4H).

### Example 70

### 4-((3aS,7aS)-3-(4-cyano-3-(trifluoromethyl)phenyl)-2-oxooctahydro-1H-benzo[d]imidazol-1-yl)-2-methylbenzamide

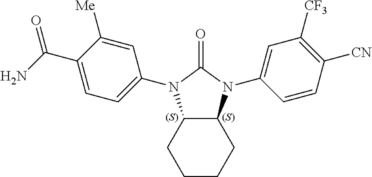

4-((3aS,7aS)-3-(4-cyano-3-(trifluoromethyl)phenyl)-2-oxooctahydro-1H-benzo[d]imidazol-1-yl)-2-methylbenzoic acid (Enantiopure) (0.060 g, 0.14 mmol) was reacted with ammonium chloride (0.020 g, 0.41 mmol) as described for the synthesis of example 18 to give a residue. The residue was purified by preparative TLC (hexane/ethyl acetate=11) to give title compound (0.011 g, 22%) as a white solid. HPLC: 96.67%; LCMS: m/z 443.4 [M+H]⁺; ¹H NMR (400 MHz, CDCl₃) δ 7.84 (d, 1H), 7.76 (d, 1H), 7.56-7.52 (m, 2H), 7.14-7.10 (m, 2H), 5.54 (bd, 2H), 3.74-3.71 (m, 2H), 2.54 (s, 3H), 2.40 (d, 1H), 2.30 (d, 1H), 2.05-2.04 (m, 2H), 1.65-1.52 (m, 4H).

### Intermediate 6: 3-Bromotetrahydro-4H-pyran-4-one (±)

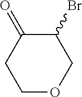

To a solution of tetrahydro-4H-pyran-4-one (8.00 g, 80.00 mmol) in diethyl ether (250 mL) was added ammonium acetate (0.61 g, 8.00 mmol) followed by NBS (14.95 g, 84.00 mmol) at 0° C. under N₂ atmosphere. After stirring for 12 h at RT, the reaction mixture was filtered, and the filtrate was concentrated under reduced pressure to give a black residue. The residue was purified by column chromatography on silica gel (hexanes/diethyl ether=9010 to 8515) to give the title compound (8.00 g, 55.0%) as a colorless semisolid. ¹H NMR (400 MHz, CDCl₃) δ 4.49-4.46 (m, 1H), 4.32-4.28 (m, 1H) 4.13-4.07 (m, 1H), 3.97-3.87 (m, 2H), 3.02-2.96 (m, 1H), 2.69-2.61 (m, 1H).

### Intermediate 7: 3-Bromo-N-(4-methoxybenzyl)tetrahydro-2H-pyran-4-amine(±)

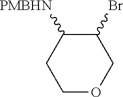

To a solution of 3-bromodihydro-2H-pyran-4(3H)-one [racemic (±)] (8.0 g, 44.69 mmol) and 4-methoxybenzyl amine (6.13 g, 44.69 mmol) in dichloroethane (100 mL) was added acetic acid (7.31 g, 134.08 mmol) at 0° C. under N₂ atmosphere. After stirring for 10 min at the same temperature, sodium triacetoxyborohydride (13.26 g, 62.57 mmol) was added, and the resulting reaction mixture was allowed to warm to RT. After stirring for 12 h at RT, the reaction mixture was diluted with water (50 mL), and extracted with dichloromethane (100 mL×2). The combined organic layer was washed with water (100 mL) followed by brine (100 mL×2), dried over Na₂SO₄, and concentrated under reduced pressure to give a residue. The crude residue was purified by column chromatography on silica gel (dichloromethane/methanol=1000 to 982) to give the title compound (6.90 g, 51.5%) as an oil. ¹H NMR (400 MHz, DMSO-d6) δ 7.24 (d, 2H), 6.85 (d, 2H), 4.7 (s, 1H), 3.99-3.94 (m, 1H), 3.86-3.82 (m, 1H), 3.67-3.56 (m, 5H), 3.60-3.56 (m, 1H), 3.40-3.15 (m, 2H), 2.63 (bs, 1H), 1.63-1.54 (m, 2H).

### Intermediate 8: Trans-3-azido-N-(4-methoxybenzyl)tetrahydro-2H-pyran-4-amine (±)

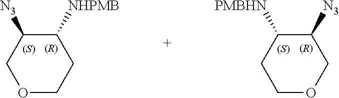

To a solution of 3-bromo-N-(4-methoxybenzyl)tetrahydro-2H-pyran-4-amine (6.50 g, 21.67 mmol) in DMSO (60 mL) was added sodium azide (2.11 g, 32.50 mmol) at RT under N₂ atmosphere and the resulting reaction mixture was heated to 90° C. for 12 h. The reaction mixture was cooled to RT, diluted with water (50 mL), and extracted with ethyl acetate (100 mL×3). The combined organic layer was washed with water (100 mL) followed by brine (100 mL), dried over Na₂SO₄, and concentrated in vacuo. The residue was purified by column chromatography on silica gel (dichloromethane/methanol=1000 to 982) to give the title compound (3.50 g, 61.7%) as an oil. LCMS: m/z 263 [M+H]⁺.

### Intermediate 9: Trans-N⁴-(4-methoxybenzyl)tetrahydro-2H-pyran-3,4-diamine (±)

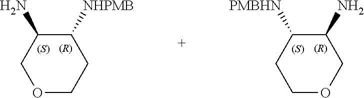

To a solution of trans-3-azido-N-(4-methoxybenzyl)tetrahydro-2H-pyran-4-amine [racemic (±)] (4.00 g, 15.21 mmol) in THF:H₂O (8:2, 30 mL) was added triphenylphosphine (5.98 g, 22.81 mmol) at RT and the resulting reaction mixture was stirred for 12 h. The reaction mixture was concentrated under reduced pressure to give a residue. The residue was dissolved in water/ethyl acetate (1:1, 100 mL) and extracted with ethyl acetate (50 mL×2). The combined organic layer was washed with water (100 mL) followed by brine (100 mL), dried over Na₂SO₄, and concentrated under reduced pressure to give a residue. The residue was purified by column chromatography on silica gel (previously impregnated with triethylamine) using dichloromethane/methanol (98:2 to 90:10) to give the title compound (1.10 g, 31.4%) as black oil. LCMS: m/z 237 [M+H]⁺; ¹H NMR (300 MHz, DMSO-d6) δ 7.26 (d, 2H), 6.87 (d, 2H), 3.77 (d, 2H), 3.72-3.69 (m, 5H), 3.56 (m, 1H), 3.21 (t, 1H), 2.87 (t, 1H), 2.49 (s, 1H), 2.45-2.35 (m, 1H), 2.30-2.15 (m, 1H), 1.95-1.84 (m, 1H), 1.22 (m, 2H).

### Intermediate 10: Trans-1-(4-Methoxybenzyl) hexahydropyrano[3,4-d]imidazol-2(3H)-one (±)

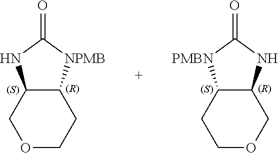

To a solution of trans-N⁴-(4-methoxybenzyl)tetrahydro-2H-pyran-3,4-diamine [racemic (±)] (1.20 g, 5.08 mmol) and triethylamine (3.54 mL, 25.42 mmol) in THF (20 ml) was added triphosgene (1.50 g, 5.08 mmol) at RT under N₂ atmosphere and the resulting reaction mixture was stirred at RT for 12 h. The reaction mixture was diluted with water (10 mL) and concentrated under reduced pressure to give a residue. The residue was again diluted with water (10 mL) and extracted with ethyl acetate (50 mL×2). The combined organic layer was washed with water (100 mL) followed by brine (100 mL), dried over Na₂SO₄, and concentrated under reduced pressure to give a residue. The residue was purified by column chromatography on silica gel (dichloromethane/methanol=98:2) to give the title compound (0.40 g, 35.9%) as a brown solid. LCMS: m/z 263 [M+H]⁺; ¹H NMR (400 MHz, CDCl₃) δ 7.28 (t, 2H), 6.88 (d, 2H), 4.75 (s, 1H), 4.40 (d, 1H), 4.20 (d, 1H), 4.10-3.95 (m, 2H), 3.79 (s, 3H), 3.25-3.16 (m, 3H), 2.92-2.88 (m, 1H), 1.25-1.17 (m, 2H).

### Intermediate 11: Trans-4-{1-[(4-Methoxyphenyl)methyl]-2-oxo-octahydropyrano[3,4-d]imidazolidin-3-yl}-2-(tri-fluoromethyl)benzonitrile (±)

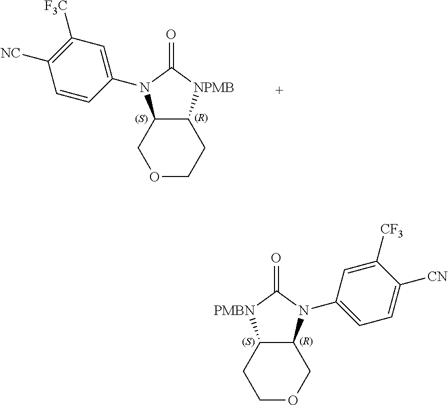

A suspension of trans-1-(4-methoxybenzyl)hexahydropyrano[3,4-d]imidazol-2(3H)-one [racemic (±)] (0.25 g, 0.95 mmol), 4-iodo-2-(trifluoromethyl)benzonitrile (0.28 g, 0.95 mmol), trans-N,N′-dimethylcyclohexane-1,2-diamine (0.032 g, 0.29 mmol) and potassium carbonate (0.395 g, 2.86 mmol) in toluene (15 mL) was degassed for 30 min in a microwave vial. CuI (0.009 g, 0.05 mmol) was added and the vial was sealed with an aluminum cap. The sealed vial was kept in a preheated oil bath at 110° C. and stirred for 12 h. The reaction mixture was cooled to RT, filtered through a pad of celite, and filtrates were concentrated under reduced pressure to give a black residue. The residue was purified by column chromatography on silica gel (dichloromethane/methanol=100:0 to 99:1) to give the title compound (0.17 g, 41.0%) as an off white solid. LCMS: m/z 432.1 [M+H]⁺; ¹H NMR (400 MHz, CDCl₃) δ 7.80-7.74 (m, 2H), 7.54 (d, 1H), 7.20 (d, 2H), 6.84 (d, 2H), 4.50 (d, 1H), 4.40-4.30 (m, 2H), 4.15-4.05 (m, 1H), 3.80 (s, 3H), 3.65 (ddd, 1H), 3.50-3.20 (m, 2H), 3.10 (ddd, 1H), 1.90 (d, 1H), 1.72-1.68 (m, 1H).

### Intermediate 12: Trans-4-(2-Oxohexahydropyrano[3,4-d]imidazol-3(2H)-yl)-2-(trifluoromethyl)benzonitrile (±)

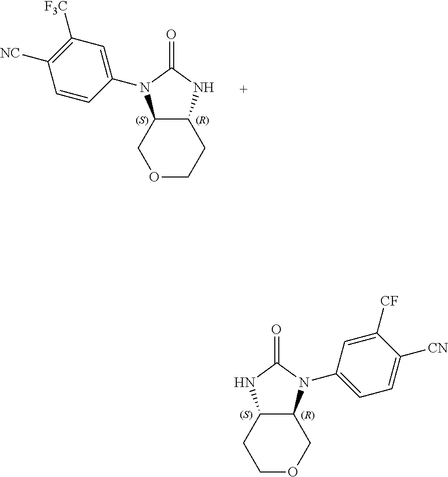

To a solution of trans-4-(1-(4-methoxybenzyl)-2-oxohexahydropyrano[3,4-d]imidazol-3(2H)-yl)-2-(trifluoromethyl)benzonitrile [racemic (±)] (0.16 g, 0.37 mmol) in CH₃CN:H₂O (1:1, 15 mL) at 0° C. was added CAN (0.61 g, 1.11 mmol) and the resulting reaction mixture was stirred for 1 h at the same temperature. The reaction mixture was diluted with water (10 mL) and concentrated under reduced pressure. The aqueous phase was extracted with ethyl acetate (20 mL×2). The combined organic layer was washed with water (50 mL) followed by brine (50 mL), dried over Na₂SO₄, and concentrated under reduced pressure to give a residue. The residue was purified by column chromatography on silica gel (dichloromethane/methanol=97:3) to give the title compound (0.060 g, 69.3%) as an off white solid. LCMS: m/z 312.2 [M+H]⁺; ¹H NMR (400 MHz, CDCl₃) δ 7.84 (d, 1H), 7.75 (d, 1H), 7.57 (d, 1H), 5.15 (s, 1H), 4.39 (dd, 1H), 4.20 (dd, 1H), 3.83 (ddd, 1H), 3.60-3.56 (m, 2H), 3.44 (t, 1H), 2.18-2.00 (m, 2H).

### Example 71

### Trans-4-{3-[4-Cyano-3-(trifluoromethyl)phenyl]-2-oxo-octahydropyrano[3,4-d]imidazolidin-1-yl}-2-fluoro-N-methylbenzamide (±)

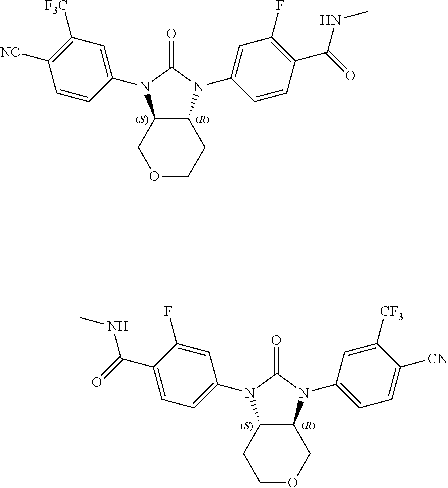

Trans-4-(2-oxohexahydropyrano[3,4-d]imidazol-3(2H)-yl)-2-(trifluoromethyl)benzonitrile [racemic (±)] (0.05 g, 0.16 mmol) was reacted with 2-fluoro-4-iodo-N-methylbenzamide (0.04 g, 0.16 mmol) as described for the synthesis of example 1 to give a residue. The residue was purified by preparative TLC using hexanes:ethyl acetate (1:1) as a mobile phase to give the title compound (0.025 g, 33.6%) as a white solid. HPLC: 95.86%; LCMS: m/z 463.1 [M+H]⁺; ¹H NMR (300 MHz, CDCl₃) δ 8.19 (m, 1H), 7.90 (d, 1H), 7.8 (d, 1H), 7.61-7.58 (m, 1H), 7.28-7.18 (m, 1H) 7.10 (dd, 1H), 6.70 (bs, 1H), 4.47 (d, 1H), 3.99-3.96 (m, 2H), 3.75-3.74 (m, 1H), 3.65-3.55 (m, 1H) 3.05 (d, 3H), 2.36 (d, 1H), 2.06-1.85 (m, 2H). The enantiomeric mixture was separated by preparative Chiral HPLC to give 71a Trans-4-{3-[4-Cyano-3-(trifluoromethyl)phenyl]-2-oxo-octahydropyrano[3,4-d]imidazolidin-1-yl}-2-fluoro-N-methylbenzamide (+) [0.090 g, retention time: 3.904 min., [α]_{D}²⁵=+78 (c=0.094, MeOH), HPLC: 99%] and 71b Trans-4-{3-[4-Cyano-3-(trifluoromethyl)phenyl]-2-oxo-octahydropyrano[3,4-d]imidazolidin-1-yl}-2-fluoro-N-methylbenzamide (−) [0.095 g, retention time: 5.877 min., [α]_{D}²⁵=78 (c=0.094, MeOH), HPLC: 99%] as white solid.

Column: LUX AMYLOSE-2 AXIA PACKED (21.2×250×5 u); Mobile Phase: n-hexane:ethanol:: 50:50 (isocratic); Flow: 20 mL/Min.

### Example 72

### Trans-4-(1-(2-methylpyridin-4-yl)-2-oxohexahydropyrano[3,4-d]imidazol-3(2H)-yl)-2-(trifluoromethyl)benzonitrile (±)

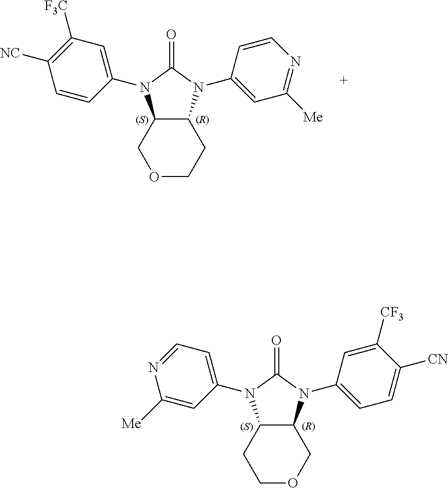

Trans-4-(2-oxohexahydropyrano[3,4-d]imidazol-3(2H)-yl)-2-(trifluoromethyl)benzonitrile [racemic (±)] (0.10 g, 0.32 mmol) was reacted with 4-bromo-2-methylpyridine (0.06 g, 0.32 mmol) as described for the synthesis of example 1 to give a residue. The residue was purified by preparative TLC (hexanes/ethyl acetate=37) to give the title compound (0.015 g, 11.5%) as a white solid. HPLC: 94.3%; LCMS: m/z 403.1 [M+H]⁺; ¹H NMR (300 MHz, DMSO-d6) δ 8.68 (d, 1H), 8.24 (d, 1H), 7.97 (d, 1H), 7.83-7.78 (m, 3H), 4.38 (d, 1H), 4.28-4.25 (m, 2H), 4.18-4.14 (m, 1H), 3.72 (m, 1H), 3.54-3.50 (m, 1H), 2.69 (s, 3H), 2.58-2.49 (m, 2H).

### Intermediate 13: Trans-2-fluoro-4-(1-(4-methoxybenzyl)-2-oxohexahydropyrano[3,4-d]imidazol-3(2H)-yl)-N-methylbenzamide (±)

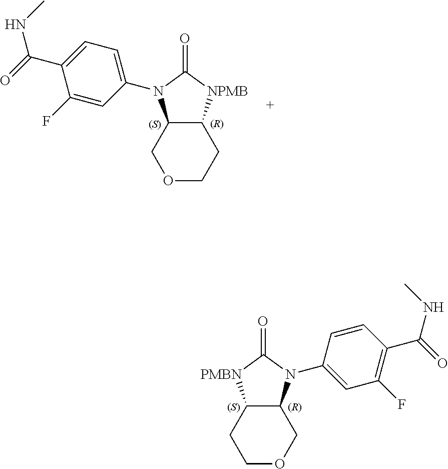

Trans-1-(4-methoxybenzyl) hexahydropyrano[3,4-d]imidazol-2(3H)-one (0.20 g, 0.76 mmol) was reacted with 2-fluoro-4-iodo-N-methylbenzamide [racemic (±)] (0.21 g, 0.76 mmol) as described for the synthesis of example 1 to give a residue. The residue was purified by column chromatography on silica gel (dichloromethane/methanol=100:0 to 99:1) to give the title compound (0.12 g, 38.0%) as an off white solid. LCMS: m/z 414.1 [M+H]⁺; ¹H NMR (300 MHz, CDCl₃) δ 8.24-8.21 (t, 1H), 7.27-7.309 (m, 3H), 7.10 (dd, 1H), 6.89 (dd, 2H), 6.67 (bs, 1H), 4.55 (d, 1H), 4.34 (t, 2H), 4.05 (dd, 1H), 3.81 (s, 3H), 3.66 (ddd, 1H), 3.46-3.36 (m, 2H), 3.15 (ddd, 1H), 3.03 (d, 3H), 1.95-1.75 (m, 2H).

### Intermediate 14: Trans-2-fluoro-N-methyl-4-(2-oxohexahydropyrano[3,4-d]imidazol-3(2H)-yl)benzamide (±)

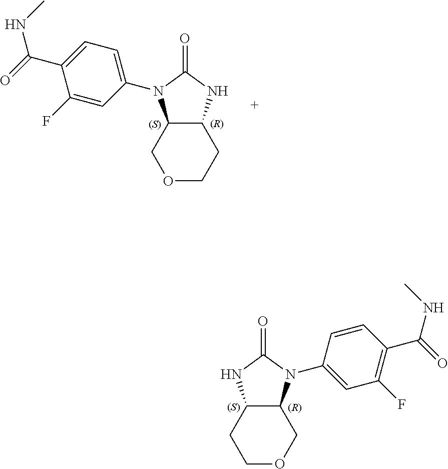

To a solution of trans-2-fluoro-4-(1-(4-methoxybenzyl)-2-oxohexahydropyrano[3,4-d]imidazol-3(2H)-yl)-N-methylbenzamide [racemic (±)] (0.120 g, 0.29 mmol) in CH₃CN:H₂O (1:1, 5 mL) at 0° C. was added CAN (0.477 g, 0.87 mmol) and the resulting reaction mixture was stirred for 1 h at the same temperature. The reaction mixture was diluted with water (10 mL), concentrated under reduced pressure, and the aqueous layer was extracted with ethyl acetate (25 mL×2). The combined organic layer was washed with water (50 mL) followed by brine (50 mL), dried over Na₂SO₄, and concentrated under reduced pressure to give a residue. The residue was purified by preparative TLC (hexane/ethyl acetate=11) to give the title compound (0.035 g, 35.0%) as a white solid.

### Example 73

### Trans-4-(1-(4-Cyano-3-(trifluoromethyl)phenyl)-2-oxohexahydropyrano[3,4-d]imidazol-3(2H)-yl)-2-fluoro-N-methylbenzamide (±)

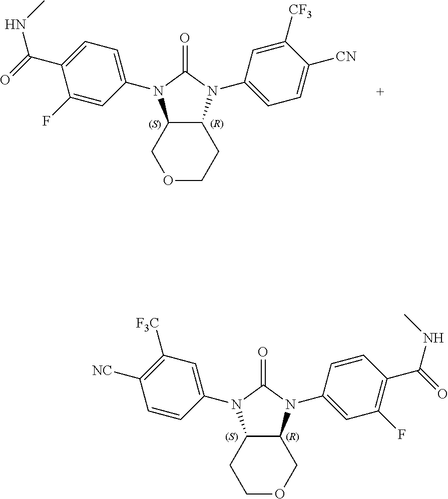

Trans-2-fluoro-N-methyl-4-(2-oxohexahydropyrano[3,4-d]imidazol-3(2H)-yl)benzamide [racemic (±)] (0.05 g, 0.17 mmol) was reacted with 4-iodo-2-(trifluoromethyl)benzonitrile (0.05 g, 0.17 mmol) as described for the synthesis of example 1 to give a residue. The residue was purified by preparative TLC (hexanes/ethyl acetate=11) to afford the title compound (0.025 g, 33.0%) as a white solid. HPLC: 95.8%; LCMS: m/z 463 [M+H]⁺; ¹H NMR (300 MHz, CDCl₃) δ 8.18 (t, 1H), 7.88 (d, 1H), 7.74 (d, 1H), 7.57 (dd, 1H), 7.17-7.10 (m, 2H), 6.65 (bs, 1H), 4.48 (d, 1H), 4.25 (dd, 1H), 3.98-3.95 (m, 2H), 3.75 (t, 1H), 3.57 (t, 1H), 3.03 (d, 3H), 2.32 (d, 1H), 2.04-1.90 (m, 1H).

### Example 74

### Trans-ethyl 4-(3-(4-cyano-3-(trifluoromethyl)phenyl)-2-oxohexahydropyrano[3,4-c]imidazol-1(6H)-yl)-2-fluorobenzoate (±)

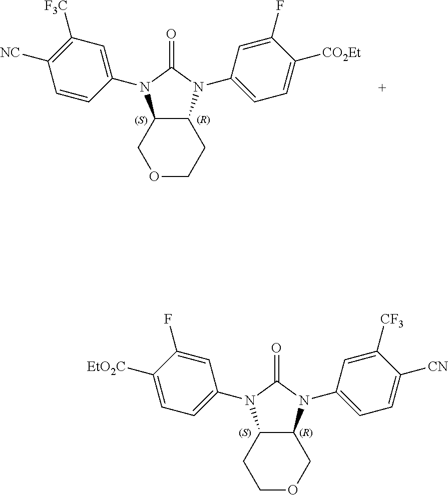

Trans-4-(2-oxohexahydropyrano[3,4-d]imidazol-3(2H)-yl)-2-(trifluoromethyl)benzonitrile [racemic (±)] (0.20 g, 0.642 mmol) was reacted with ethyl 2-fluoro-4-iodobenzoate (0.188 g, 0.642 mmol) as described for the synthesis of example 1 to give a residue. The residue was purified by column chromatography on silica gel (dichloromethane/methanol=1000 to 99.5/0.5%) to give the the title compound (0.120 g, 39%) as a white solid. LCMS: m/z 478.4 [M+H]⁺.

### Example 75

### Trans-4-(3-(4-cyano-3-(trifluoromethyl)phenyl)-2-oxohexahydropyrano[3,4-d]imidazol-1(6H)-yl)-2-fluorobenzoic acid (±)

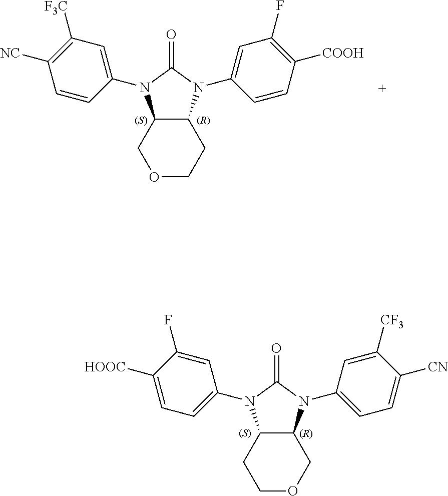

To a solution of trans-ethyl 4-(3-(4-cyano-3-(trifluoromethyl)phenyl)-2-oxohexahydropyrano[3,4-d]imidazol-1(6H)-yl)-2-fluorobenzoate [racemic (±)] (0.120 g, 0.251 mmol) in THF (10 mL) was added aqueous solution of lithium hydroxide monohydrate (0.0126 g, 0.301 mmol) as described for synthesis of example 18 to give a residue. The residue was triturated with ether to give the title compound (0.060 g, 53.0%) as an off white solid. LCMS: m/z 448.4 [M−H]⁺; ¹H NMR (400 MHz, DMSO-d₆) δ 13.2 (bs, 1H), 8.20 (d, 1H), 7.97-7.90 (m, 2H), 7.78 (m, 1H), 7.38-7.30 (m, 2H), 4.37 (d, 1H), 4.18-4.09 (m, 3H), 3.73-1.68 (m, 1H), 3.52-3.47 (m, 1H), 2.32 (d, 1H), 1.78-1.74 (m, 1H).

### Example 76

### Trans-4-(3-(4-cyano-3-(trifluoromethyl)phenyl)-2-oxohexahydropyrano[3,4-d]imidazol-1(6H)-yl)-2-fluorobenzamide (±)

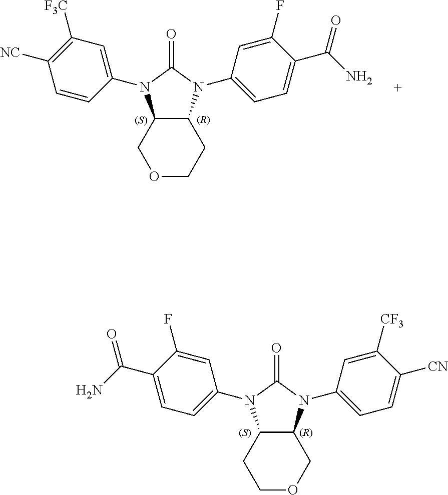

Trans-4-(3-(4-cyano-3-(trifluoromethyl)phenyl)-2-oxohexahydropyrano[3,4-d]imidazol-1(6H)-yl)-2-fluorobenzoic acid [racemic (±)] (0.090 g, 0.200 mmol) was reacted with ammonium chloride (0.021 g, 0.40 mmol) as described for the synthesis of example 18 to give a residue. The residue was purified by preparative TLC (hexanes/ethyl acetate=1/1) to give the title compound (0.025 g, 28%) as a white solid. HPLC: 99.4%; LCMS: m/z 448.8 [M+H]⁺; ¹H NMR (400 MHz, DMSO-d₆) δ 8.19 (t, 1H), 7.97 (d, 1H), 7.80-7.65 (m, 4H), 7.35-7.26 (m, 2H), 4.38 (d, 1H), 4.16-4.08 (m, 3H), 3.73-3.67 (m, 1H), 3.52-3.48 (m, 1H), 2.33-2.26 (m, 1H), 1.78-1.74 (m, 1H). The enantiomeric mixture was separated by chiral HPLC to give 76a [0.009 g, retention time: 13.977 min., HPLC: 96.71%] and 76b [0.005 g, retention time: 18.426 min., HPLC: 96.24%] as white solid.

Column: LUX AMYLOSE (21.2×250×5 u); Mobile Phase: n-hexane:ethanol:: 50:50 (isocratic);

Flow: 20 mL/Min.

| TIME | % B | FLOW (mL/min) |
|---|---|---|
| 0.0 | 35.0 | 20.0 |

### Example 77

### Trans-ethyl-4-(-3-(4-cyano-3-(trifluoromethyl)phenyl)-2-oxohexahydropyrano[3,4-d]imidazol-1(6H)-yl)-2-methyl benzoate (±)

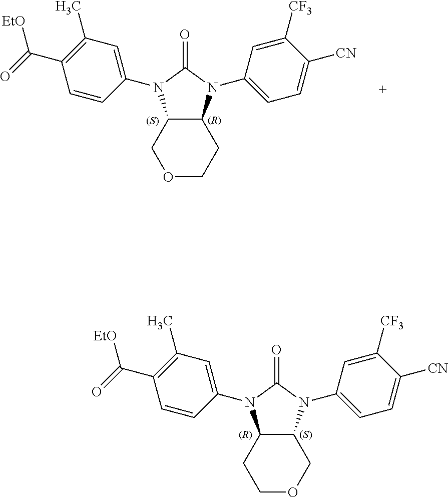

Trans-4-(2-oxohexahydropyrano[3,4-d]imidazol-3(2H)-yl)-2-(trifluoromethyl)benzonitrile [racemic (±)] (0.200 g, 0.642 mmol) was reacted with ethyl 4-iodo2-methyl benzoate (0.186 g, 0.642 mmol) as described for the synthesis of example 1 to give a residue. The residue was purified by column chromatography on silica gel (dichloromethane/methanol=955) to give the title compound (0.05 g, 16%) as a white solid. LCMS: m/z 474.5 [M+H]⁺; ¹H NMR (400 MHz, DMSO-d₆) δ 8.01 (d, 1H), 7.85 (d, 1H), 7.80 (bs, 1H), 7.62-7.57 (m, 1H), 4.5 (d, 1H), 4.41-4.23 (m, 3H), 3.97 (m, 2H), 2.63 (s, 3H), 2.30 (m, 1H), 2.00-1.90 (m, 1H).

### Example 78

### Trans-4-(3-(4-cyano-3-(trifluoromethyl)phenyl)-2-oxohexahydropyrano[3,4-d]imidazol-1(6H)-yl)-2-methylbenzoic acid (±)

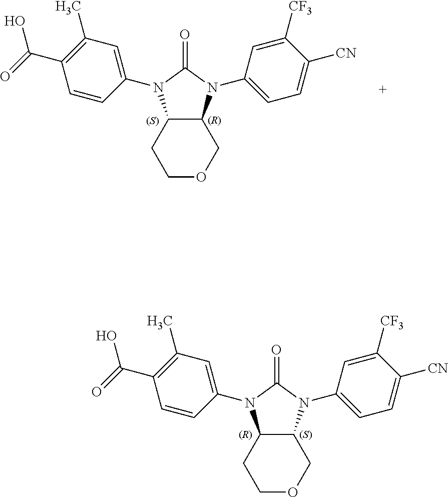

Trans-ethyl-4-(3-(4-cyano-3-(trifluoromethyl)phenyl)-2-oxohexahydropyrano[3,4-d]imidazol-1(6H)-yl)-2-methylbenzoate [racemic (±)] (0.050 g, 0.105 mmol) in THF (5 mL) was reacted with aqueous lithium hydroxide monohydrate (0.004 g, 0.105 mmol) as described for synthesis of example 17 to give a residue. LCMS: m/z 446 [M+H]⁺. The crude product was used as such for the next step without further purification.

### Example 79

### Trans-4-(3-(4-cyano-3-(trifluoromethyl)phenyl)-2-oxohexahydropyrano[3,4-c]imidazol-1(6H)-yl)-2-methylbenzamide (±)

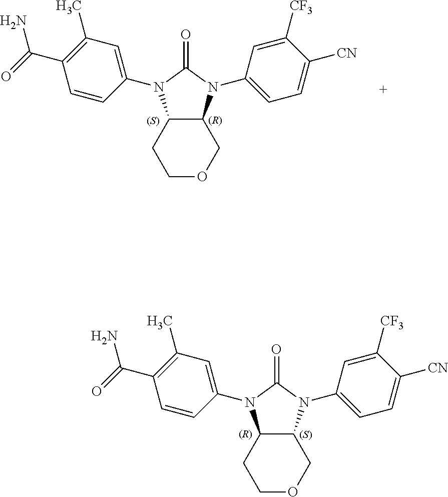

Trans-4-(3-(4-cyano-3-(trifluoromethyl)phenyl)-2-oxohexahydropyrano[3,4-d]imidazol-1(6H)-yl)-2-methyl benzoic acid [racemic (±)] (0.046 g, 0.103 mmol) was reacted with ammonium chloride (0.011 g, 0.206 mmol) as described for the synthesis of example 18 to give a residue. The residue was purified by preparative TLC (dichloromethane/methanol=955) to give the title compound (0.020 g, 43%) as a white solid. HPLC: 98.33%; LCMS: m/z 444.8 [M+H]⁺; ¹H NMR (400 MHz, DMSO-d₆) δ 8.18 (d, 1H), 7.97 (bs, 1H), 7.78-7.74 (m, 2H), 7.18-7.21 (m, 2H), 4.39 (d, 1H), 4.11-4.07 (m, 3H), 3.70-3.65 (m, 1H), 3.52-3.48 (m, 1H), 2.39 (s, 3H), 2.17-2.14 (m, 1H), 1.80-1.70 (m, 1H).

Column: AGC1815-011

Mobile phase A: 0.01% TFA IN WATER; B: ACN: MeOH (1:1); Gradient: 70:30; Flow: 1 mL/min; Temperature: 40° C.

### Intermediate 15: Trans-4-(2-aminocycloheptyl)amino)-2-(trifluoromethyl)benzonitrile (±)

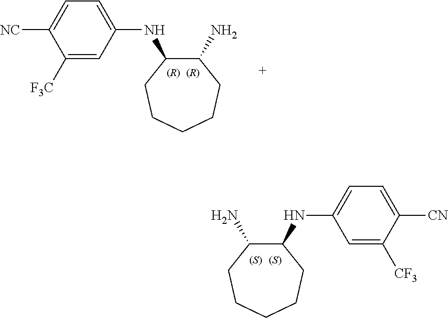

To a solution of trans-1,2-diaminocycloheptane dihydrochloride [racemic (±)] (1.0 g, 4.97 mmol) in DMSO (25 mL) under argon atmosphere was added triethylamine (1.37 mL, 9.94 mmol) followed by 4-fluoro-2-(trifluoromethyl)benzonitrile (0.845 g, 4.47 mmol) at RT and the resulting reaction mixture was heated to 45° C. After stirring for 16 h, the reaction mixture was cooled to RT, poured onto ice-water (50 mL), basified with 1N NaOH solution, and extracted with ethyl acetate (100 mL×2). The combined organic layer was washed with water (50 mL) followed by brine (50 mL), dried over Na₂SO₄, and concentrated under reduced pressure to give the title compound (1.1 g) as a brown residue. The crude residue was directly used for next step without further purification.

### Intermediate 16: Trans-4-(2-oxooctahydrocyclohepta[d]imidazol-1(2H)-yl)-2-(trifluoromethyl)benzonitrile (±)

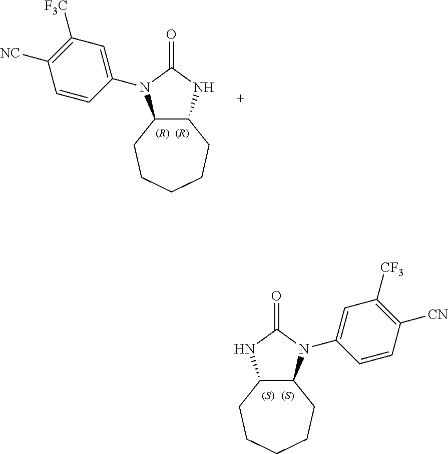

To a solution of trans-4-(2-aminocycloheptyl)amino)-2-(trifluoromethyl)benzonitrile [racemic (±)] (1.10 g, 3.70 mmol) in THF (15 mL) was added triethylamine (1.53 mL, 11.07 mmol) followed by 1,1′-carbonyldiimidazole (1.19 g, 7.399 mmol) at RT under N₂ atmosphere. After stirring for 2 h, the reaction mixture was quenched by adding water (10 mL), and concentrated under reduced pressure. The aqueous layer was further diluted with water (50 mL) and extracted with ethyl acetate (50 mL×2). The combined organic layer was washed with water (50 mL) followed by brine (50 mL), dried over Na₂SO₄, and concentrated under reduced pressure to give a residue. The crude residue was purified by column chromatography on silica gel (dichloromethane/methanol=991) to give the title compound (0.15 g, 13.0%) as a white solid. LCMS: m/z 323 [M+H]⁺; ¹H NMR (400 MHz, CDCl₃) δ 7.80 (d, 1H), 7.70 (d, 1H), 7.56 (dd, 1H), 4.98 (s, 1H), 4.3\tilde{5}4.05 (m, 1H), 3.75 (t, 1H), 2.35-2.25 (m, 1H), 2.19-2.10 (m, 1H), 1.85-1.61 (m, 8H).

### Example 80

### Trans-4-(3-(4-cyano-3-(trifluoromethyl)phenyl)-2-oxooctahydrocyclohepta[d]imidazol-1(2H)-yl)-2-fluoro-N-methylbenzamide (±)

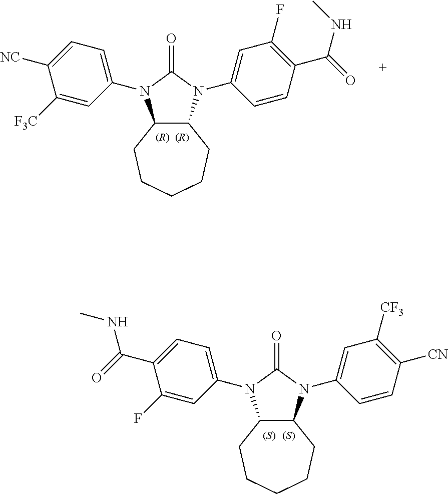

Trans-4-(2-oxooctahydrocyclohepta[d]imidazol-1(2H)-yl)-2-(trifluoromethyl)benzonitrile [racemic (±)] (0.150 g, 0.464 mmol) was reacted with 2-fluoro-4-iodo-N-methylbenzamide as described for the synthesis of example 1 (0.155 g, 0.557 mmol) to give a residue. The residue was purified by column chromatography on silica gel (hexanes/ethyl acetate=11) to give the title compound (0.075 g, 38%) as an off white solid. HPLC=96%; LCMS: m/z 475 [M+H]⁺; ¹H NMR (400 MHz, CDCl₃) δ 8.16 (t, 1H), 7.85 (d, 1H), 7.84 (s, 1H), 7.58 (dd, 1H), 7.28 (dd, 1H), 7.10 (dd, 1H), 6.71 (dd, 1H), 4.20 (d, 2H), 3.05 (d, 3H), 2.40 (m, 2H), 1.90-1.78 (m, 4H), 1.76-1.68 (m, 2H), 1.58-1.48 (m, 2H); Chiral HPLC=47.91:48.81. The enantiomeric mixture was separated by preparative Chiral HPLC to give 80a Trans-4-(3-(4-cyano-3-(trifluoromethyl)phenyl)-2-oxooctahydrocyclohepta[d]imidazol-1(2H)-yl)-2-fluoro-N-methylbenzamide (−) [0.018 g, retention time: 10.39 min., [α]_{D}²⁵=105 (c=0.104, CHCl₃), HPLC: 91.43%] and 80b Trans-4-(3-(4-cyano-3-(trifluoromethyl)phenyl)-2-oxooctahydrocyclohepta[d]imidazol-1(2H)-yl)-2-fluoro-N-methylbenzamide (+) [0.017 g, retention time: 18.69 min., [α]_{D}²⁵=+106 (c=0.102, CHCl₃), HPLC: 93.73%] as white solids.

### Intermediate 17: Trans-tert-butyl-8-hydroxy-1,4-dioxaspiro[4.5]decan-7-yl)carbamate (±)

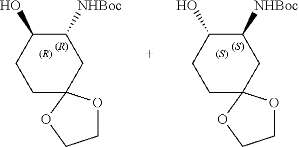

To a solution of trans-7-amino-1,4-dioxaspiro[4.5]decan-8-ol [racemic (±)] (0.530 g, 3.06 mmol) in DCM (5 mL) was added NEt₃ (0.85 mL, 6.12 mmol) followed by (Boc)₂O (0.73 mL, 3.37 mmol) at RT. After stirring for 2 h, the reaction mixture was quenched by adding water (10 mL), and extracted with DCM (10 mL×2). The combined organic layer was dried over Na₂SO₄, and concentrated under reduced pressure to give the title compound (0.500 g, crude) as a white solid. LCMS: m/z 174.0 [M-100]⁺; ¹H NMR (300 MHz, CDCl₃) δ 5.05 (s, 1H), 3.95 (d, 4H), 3.70 (s, 1H), 3.55 (m, 1H), 2.75 (s, 1H), 2.13-2.08 (m, 1H), 2.00-1.90 (m, 1H), 1.75-1.85 (m, 1H), 1.67-1.70 (m, 1H), 1.52-1.55 (m, 2H), 1.44 (s, 9H).

### Intermediate 18: tert-butyl(8-oxo-1,4-dioxaspiro[4.5]decan-7-yl)carbamate (±)

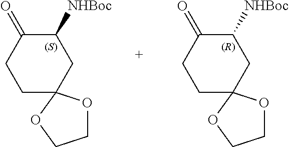

To a solution of trans-tert-butyl-8-hydroxy-1,4-dioxaspiro[4.5]decan-7-yl) carbamate [racemic (±)] (0.500 g, 1.830 mmol) in DCM (10 mL) was added Dess-Martin periodinane (0.776 g, 1.830 mmol) at RT. After stirring for 30 min, the reaction mixture was quenched with NaHCO₃ solution (25 mL) and extracted with DCM (20 mL×2). The combined organic layer was dried over Na₂SO₄ and concentrated under reduced pressure to give the title compound (0.500 g, crude) as a white solid. The crude product was used as such for the next step without further purification.

### Intermediate 19: Cis- and trans-tert-butyl(8-((4-methoxybenzyl)amino)-1,4-dioxaspiro[4.5]decan-7-yl)carbamate

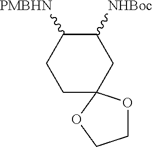

To a solution of tert-butyl(8-oxo-1,4-dioxaspiro(4.5)decan-7-yl)carbamate (0.12 g, 0.442 mmol) and 4-methoxybenzylamine (0.06 mL, 0.442 mmol) in dichloroethane (5 mL) was added acetic acid (0.078 g, 1.3 mmol) at 0° C. under N₂ atmosphere. After stirring for 10 min at the same temperature, sodium triacetoxyborohydride (0.12 g, 0.57 mmol) was added, and the resulting reaction mixture was allowed to warm to RT. After stirring for 2 h, the reaction mixture was diluted with NaHCO₃ solution (10 mL), and extracted with DCM (10 mL×2). The combined organic layer was washed with water (10 mL) followed by brine (10 mL×2), dried over Na₂SO₄, and concentrated under reduced pressure to give the title compounds (0.120 g, crude) as an oil. LCMS: m/z 393.5 [M+H]⁺.

### Intermediate 20: Cis- and trans-tert-butyl (8-amino-1,4-dioxaspiro[4.5]decan-7-yl)carbamate

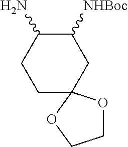 [SMILES: CC(C)(C)OC(=O)NC1CC2(CCC1N)OCCO2]

To a solution of cis- and trans-tert-butyl(8-((4-methoxybenzyl)amino)-1,4-dioxaspiro[4.5]decan-7-yl)carbamate (0.120 g, 0.306 mmol) in MeOH (50 mL) was added 10% Pd/C (0.025 g) at RT and the resulting suspension was stirred under hydrogen pressure (60 psi) for 24 h using Parr shaker. The suspension was filtered on celite pad and the filtrate was concentrated under reduced pressure to give the title compounds (0.060 g, crude). The crude product was used as such for the next step without further purification.

### Intermediate 21: Cis- and trans-tert-butyl(8-((4-cyano-3-(trifluoromethyl)phenyl)amino)-1,4-dioxaspiro[4.5]decan-7-yl)carbamate

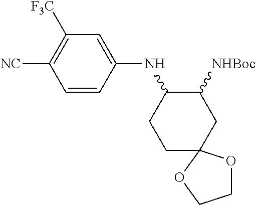 [SMILES: CC(C)(C)OC(=O)NC1CC2(CCC1Nc1ccc(C#N)c(C(F)(F)F)c1)OCCO2]

To a solution of cis- and trans-tert-butyl (8-amino-1,4-dioxaspiro[4.5]decan-7-yl)carbamate (0.250 g, 0.919 mmol) in DMSO (5 mL) under Ar atmosphere was added NEt₃ (0.130 mL, 0.919 mmol) and 4-fluoro-2-(trifluoromethyl)benzonitrile (0.173 g, 0.919 mmol) at RT. The resulting reaction mixture was heated to 45° C. and stirred for 16 h. The reaction mixture was cooled to RT, poured onto ice-water (10 mL), and extracted with ethyl acetate (10 mL×2). The combined organic layer was washed with water (10 mL×1) followed by brine (10 mL×1), dried over Na₂SO₄, and concentrated under reduced pressure to give a residue. The residue was purified by column chromatography on silica gel (hexanes/ethyl acetate=13) to give the title compounds (0.065 g, 20.0%) as a light yellow solid. LCMS: m/z 341.9 [M−100]⁺.

### Intermediate 22: Cis- and trans-4-((7-amino-1,4-dioxaspiro[4.5]decan-8-yl)amino)-2-(trifluoromethyl)benzonitrile

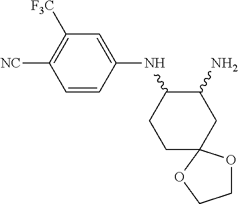 [SMILES: N#Cc1ccc(NC2CCC3(CC2N)OCCO3)cc1C(F)(F)F]

To a solution of cis- and trans-tert-butyl (8-((4-cyano-3-(trifluoromethyl)phenyl)amino)-1,4-dioxaspiro[4.5]decan-7-yl)carbamate (0.065 g, 0.147 mmol) in DCM (5 mL) was added TFA (0.3 mL) at 0° C. The resulting reaction mixture was stirred at RT for 2 h. The reaction mixture was poured onto NaHCO₃ solution (10 mL) and extracted with DCM (10 mL×2). The combined organic layer was washed with water (10 mL) followed by brine (10 mL), dried over Na₂SO₄, and concentrated under reduced pressure to give the title compounds (0.040 g, 86.0%) as a yellow solid. LCMS: m/z 341.9 [M+H]⁺.

### Intermediate 23: Cis- and trans-4-(2-oxohexahydrospiro[benzo[d]imidazole-5,2′-[1,3]dioxolan]-1(6H)-yl)-2-(trifluoromethyl)benzonitrile

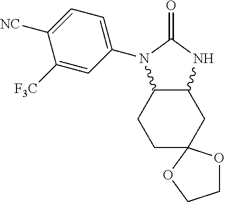 [SMILES: N#Cc1ccc(N2C(=O)NC3CC4(CCC32)OCCO4)cc1C(F)(F)F]

To a solution of cis- and trans-4-((7-amino-1,4-dioxaspiro[4.5]decan-8-yl)amino)-2-(trifluoromethyl)benzonitrile (0.040 g, 0.117 mmol) in THF (3 mL) was added NEt₃ (0.049 mL, 0.351 mmol) followed by 1,1′-carbonyldiimidazole (0.038 g, 0.234 mmol) at RT under N₂ atm. After stirring for 2 h, the reaction mixture was quenched by adding water (10 mL), and concentrated under reduced pressure. The aqueous layer was further diluted with water (10 mL) and extracted with ethyl acetate (10 mL×2). The combined organic layer was dried over Na₂SO₄ and concentrated under reduced pressure to give the title compounds (0.020 g, 46.0%) as a yellow solid. The crude product was used as such for the next step without further purification.

### Intermediates 24 and 25: Trans-4-(1-(4-cyano-3-(trifluoromethyl)phenyl)-2-oxohexahydrospiro[benzo[d]imidazole-5,2′-[1,3]dioxolan]-3(2H)-yl)-2-fluoro-N-methylbenzamide (±) and cis-4-(1-(4-cyano-3-(trifluoromethyl)phenyl)-2-oxohexahydrospiro[benzo[d]imidazole-5,2′-[1,3]dioxolan]-3(2H)-yl)-2-fluoro-N-methylbenzamide (±)

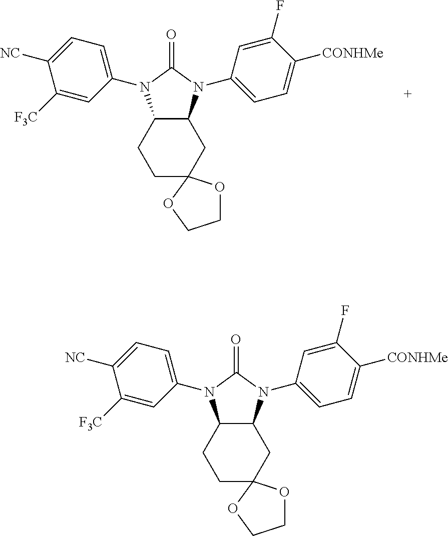

A suspension of cis- and trans-4-(2-oxohexahydrospiro[benzo[d]imidazole-5,2′-[1,3]dioxolan]-1(6H)-yl)-2-(trifluoromethyl)benzonitrile (±) (0.020 g, 0.054 mmol), 2-fluoro-4-iodo-N-methylbenzamide (0.015 g, 0.054 mmol), trans-1,2-diaminocyclohexane (±) (0.0012 g, 0.0109 mmol) and tripotassium phosphate (0.034 g, 0.163 mmol) in toluene (4 mL) was degassed for 30 min in a microwave vial. CuI (0.001 g, 0.054 mmol) was added and the vial was sealed with an aluminum cap. The sealed vial was kept in a preheated oil bath at 110° C. and stirred for 12 h. The reaction mixture was cooled to RT, filtered through a pad of celite, and the filtrate was concentrated under reduced pressure to give a residue. The residue was purified by column chromatography on silica gel (ethyl acetate/hexane=82) to give a mixture of title the compounds (0.020 g, 71.4%) as light yellow solids. The above mixture was separated by preparative TLC (hexanes/ethyl acetate=64). The nonpolar isomer (trans-4-(1-(4-cyano-3-(trifluoromethyl)phenyl)-2-oxohexahydrospiro[benzo[d]imidazole-5,2′-[1,3]dioxolan]-3(2H)-yl)-2-fluoro-N-methylbenzamide) [racemic (±)] was obtained (0.005 g, 17.8%) as an off white solid. LCMS: m/z 519.5 [M+H]⁺; ¹H NMR (400 MHz, CDCl₃) δ 8.20 (t, 1H), 7.85 (d, 1H), 7.75 (s, 1H), 7.55 (d, 1H), 7.15 (d, 1H), 7.05 (d, 1H), 6.70 (s, 1H), 4.05 (m, 1H), 4.00 (m, 4H), 3.80 (m, 1H), 3.05 (d, 3H), 2.45 (d, 1H), 2.35 (d, 1H), 2.05 (d, 1H), 1.60-1.90 (m, 3H). The polar isomer (cis-4-(1-(4-cyano-3-(trifluoromethyl)phenyl)-2-oxohexahydrospiro[benzo[d]imidazole-5,2′-[1,3]dioxolan]-3(2H)-yl)-2-fluoro-N-methylbenzamide) [racemic (±)] was obtained (0.005 g, 17.8%) as an off white solid. LCMS: m/z 519.5 [M+H]⁺; ¹H NMR (400 MHz, CDCl₃) δ 8.15 (t, 1H), 7.95 (s, 1H), 7.85 (d, 1H), 7.7 (d, 1H), 7.65 (d, 1H), 7.18 (d, 1H), 6.74 (s, 1H), 4.65 (q, 1H), 4.50 (q, 1H), 3.95 (m, 3H), 3.75 (m, 1H), 3.05 (d, 3H), 2.20 (m, 2H), 2.05 (m, 1H), 1.80 (q, 1H), 1.70 (m, 2H).

### Example 81

### Trans-4-(3-(4-cyano-3-(trifluoromethyl)phenyl)-2,6-dioxooctahydro-1H-benzo[d]imidazol-1-yl)-2-fluoro-N-methylbenzamide (±)

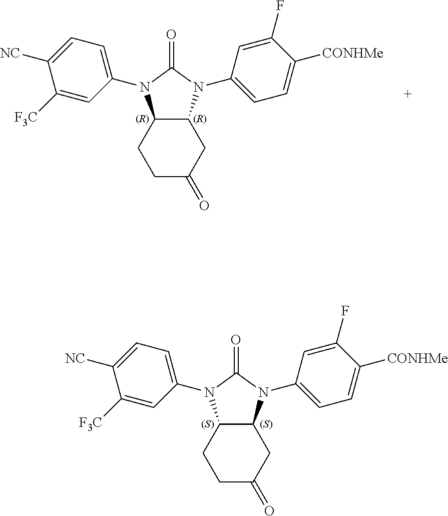

To a solution of trans-4-(1-(4-cyano-3-(trifluoromethyl)phenyl)-2-oxohexahydrospiro[benzo[d]imidazole-5,2′-[1,3]dioxolan]-3(2H)-yl)-2-fluoro-N-methylbenzamide [racemic (±)] (0.080 g, 0.154 mmol) in acetone (2 mL) was added 2N HCl (2 mL) at RT and the reaction mixture was heated to 60° C. for 2 h. The reaction mixture was poured onto ice-water (5 mL) and extracted with ethyl acetate (5 mL×2). The combined organic layer was washed with satd. NaHCO₃ (5 mL×2) followed by water (5 mL×2), dried over Na₂SO₄, and concentrated under reduced pressure to give the title compound (0.060 g, 82.0%) as an off white solid. HPLC: 97.17%; LCMS: m/z 475.5 [M+H]⁺; ¹H NMR (400 MHz, CDCl₃) δ 8.20 (t, 1H), 7.91 (d, 1H), 7.80 (s, 1H), 7.60 (d, 1H), 7.19 (d, 1H), 7.05 (d, 1H), 6.70 (s, 1H), 4.20 (m, 2H), 3.50 (m, 1H), 3.30 (m, 1H), 3.05 (d, 3H), 2.80 (m, 1H), 2.60 (m, 3H).

### Example 82

### Trans-4-(3-(4-cyano-3-(trifluoromethyl)phenyl)-6-hydroxy-2-oxooctahydro-1H-benzo[d]imidazol-1-yl)-2-fluoro-N-methylbenzamide (±)

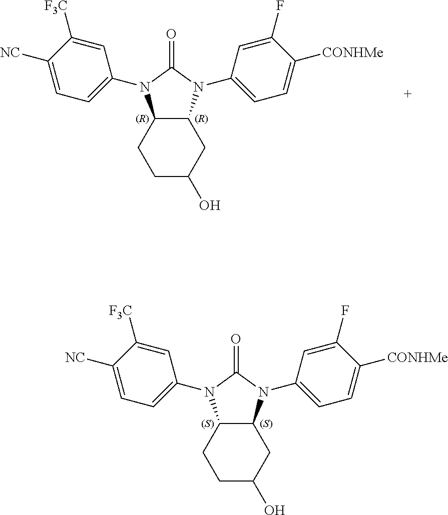

To a solution of trans-4-(3-(4-cyano-3-(trifluoromethyl)phenyl)-2,6-dioxooctahydro-1H-benzo[d]imidazol-1-yl)-2-fluoro-N-methylbenzamide [racemic (±)] (0.100 g, 0.211 mmol) in MeOH (5 mL) was added NaBH₄ (0.016 g, 0.422 mmol) portionwise at 0° C. After stirring for 30 min at the same temperature, the reaction mixture was quenched with 1N HCl (2 mL), and extracted with ethyl acetate (10 mL×2). The combined organic layer was dried over Na₂SO₄ and concentrated under reduced pressure to give the title compound (0.100 g, crude) as an off white solid. HPLC: 97.90%; LCMS: m/z 476.8 [M+H]⁺; ¹H NMR (400 MHz, CDCl₃) δ 8.20 (t, 1H), 7.88 (d, 1H), 7.75 (s, 1H), 7.55 (d, 1H), 7.19 (d, 1H), 7.05 (d, 1H), 6.70 (s, 1H), 4.10 (m, 1H), 3.80 (m, 1H), 3.70 (m, 1H), 3.05 (d, 3H), 2.75 (m, 1H), 2.40 (m, 2H), 1.90 (d, 1H), 1.7 (m, 3H). The enantiomeric mixture was separated by preparative Chiral HPLC to give 82a Trans-4-(3-(4-cyano-3-(trifluoromethyl)phenyl)-6-hydroxy-2-oxooctahydro-1H-benzo[d]imidazol-1-yl)-2-fluoro-N-methylbenzamide (+) [0.080 g, retention time: 11.944 min., [α]_{D}²⁵=+4 (c=0.108, MeOH), HPLC: 98.11%] and 82b Trans-4-(3-(4-cyano-3-(trifluoromethyl)phenyl)-6-hydroxy-2-oxooctahydro-1H-benzo[d]imidazol-1-yl)-2-fluoro-N-methylbenzamide (−) [0.080 g, retention time: 15.366 min., [α]_{D}²⁵=2 (c=0.132, MeOH), HPLC: 96.62%] as white solids. Column: LUXAMYLOSE-2 AXIA PACKED (21.2×250×5 u); Mobile phase: n-hexane:ethanol:: 75:25 (isocratic); Flow: 20 mL/min. The absolute stereochemistry of both 82a and 82b is unknown.

### Intermediate 26: Trans-1-(4-cyano-3-(trifluoromethyl)phenyl)-3-(3-fluoro-4-(methylcarbamoyl)phenyl)-2-oxooctahydro-1H-benzo[d]imidazol-5-yl methanesulfonate

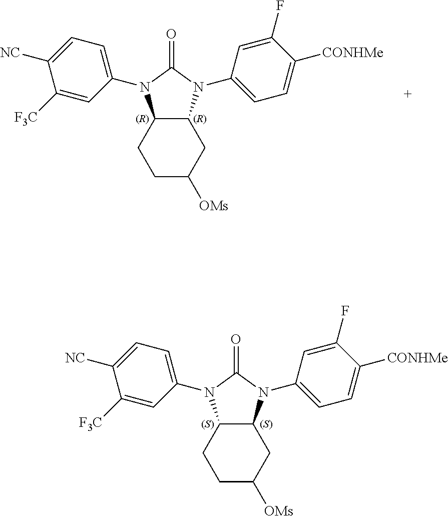

To a solution of trans-4-(3-(4-cyano-3-(trifluoromethyl)phenyl)-6-hydroxy-2-oxooctahydro-1H-benzo[d]imidazol-1-yl)-2-fluoro-N-methylbenzamide [racemic (±)] (0.100 g, 0.210 mmol) in DCM (5 mL) was added NEt₃ (0.088 mL, 0.620 mmol) followed by methanesulfonyl chloride (0.04 mL, 0.500 mmol) at 0° C. The reaction mixture was allowed to warm to RT and stirred for 2 h. The reaction mixture was poured onto NaHCO₃ solution (10 mL) and extracted with DCM (10 mL×2). The combined organic layer was dried over Na₂SO₄ and concentrated under reduced pressure to give the title compound (0.10 g, crude) as an off white solid. LCMS: m/z 554.7 [M+H]⁺.

### Examples 83 and 84

### Trans-4-(3-(4-cyano-3-(trifluoromethyl)phenyl)-2-oxo-2,3,3a,4,7,7a-hexahydro-1H-benzo[c]imidazol-1-yl)-2-fluoro-N-methylbenzamide (±) and trans-4-(3-(4-cyano-3-(trifluoromethyl)phenyl)-2-oxo-2,3,3a,4,5,7a-hexahydro-1H-benzo[d]imidazol-1-yl)-2-fluoro-N-methylbenzamide (±)

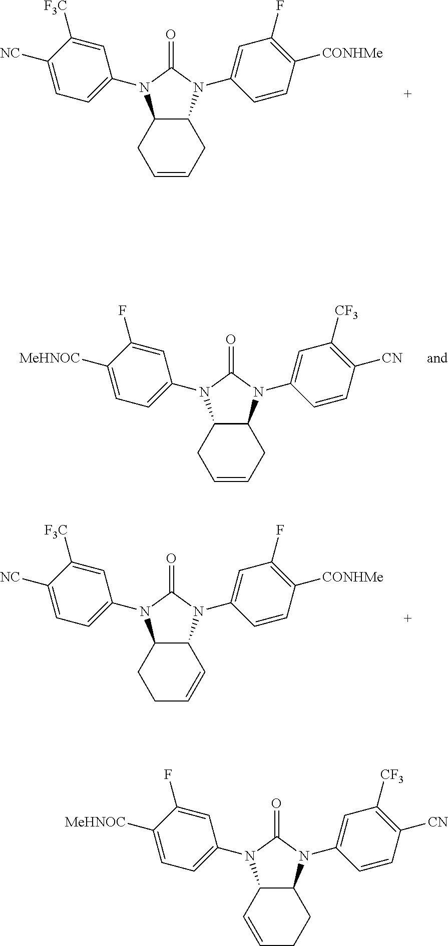

A mixture of 1-(4-cyano-3-(trifluoromethyl)phenyl)-3-(3-fluoro-4-(methylcarbamoyl)phenyl)-2-oxooctahydro-1H-benzo[d]imidazol-5-yl methanesulfonate [racemic (±)] (0.100 g, 0.180 mmol) and DBU (0.10 mL) was heated to 100° C. for 2 h. The reaction mixture was diluted with brine solution (10 mL) and extracted with ethyl acetate (10 mL×2). The combined organic layer was dried over Na₂SO₄ and concentrated under reduced pressure to give a mixture of the title compounds (0.05 g, 60.0%) as a white solid. The mixture was separated by preparative HPLC to give example 83 (0.010 g) as a white solid; HPLC: 98.50%; retention time=5.117 min; LCMS: m/z 458.8 [M+H]⁺; ¹H NMR (400 MHz, CDCl₃) δ 8.17 (t, 1H), 7.86 (d, 1H), 7.79 (s, 1H), 7.60 (d, 1H), 7.20 (d, 1H), 7.10 (d, 1H), 6.70 (s, 1H), 5.85 (s, 2H), 4.00 (m, 2H), 3.05 (d, 3H), 2.80-2.90 (m, 2H), 2.20-2.30 (m, 2H); and example 84 (0.005 g) as a white solid. HPLC: 87.0%; retention time=5.215 min; LCMS: m/z 458.8 [M+H]⁺; ¹H NMR (400 MHz, CDCl₃) δ 8.20 (t, 1H), 7.87 (d, 1H), 7.77 (s, 1H), 7.58 (d, 1H), 7.30 (d, 1H), 7.10 (d, 1H), 6.75 (s, 1H), 6.15 (d, 1H), 5.85 (d, 1H), 4.49 (d, 1H), 4.00 (ddd, 1H), 3.05 (d, 3H), 2.59 (m, 2H), 2.50 (m, 1H), 1.80 (m, 1H).

COLUMN: AG/AD/PP/C18-026

FLOW: 20 ML/MIN; A: 0.01% TFA IN WATER; B: ACN: 55:45:: A:B

### Intermediate 27: Cis- and trans-N8-(4-methoxybenzyl)-1,4-dioxaspiro[4.5]decane-7,8-diamine

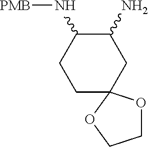

To a solution of cis- and trans-tert-butyl(8-((4-methoxybenzyl)amino)-1,4-dioxaspiro[4.5]decan-7-yl)carbamate (2.0 g, 5.100 mmol) in DCM (20 mL) was added TFA (10 mL) at 0° C. The resulting reaction mixture was warmed to RT and stirred for 2 h. The reaction mixture was poured onto NaHCO₃ solution (60 mL) and extracted with DCM (25 mL×2). The combined organic layer was washed with water (10 mL) followed by brine (10 mL), dried over Na₂SO₄, and concentrated under reduced pressure to give the title compounds (1.30 g, crude). The crude product was used as such for the next step without further purification. LCMS: m/z 293.4[M+H]⁺.

### Intermediate 28: Cis- and trans-1-(4-methoxybenzyl)hexahydrospiro[benzo[d]imidazole-5,2′-[1,3]dioxolan]-2(3H)-one

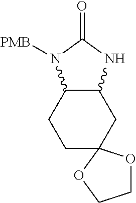

To a solution of cis- and trans-N8-(4-methoxybenzyl)-1,4-dioxaspiro[4.5]decane-7,8-diamine (1.30 g, 4.4 mmol) in THF (20 mL) was added NEt₃ (1.85 mL, 13.200 mmol) followed by 1,1′-carbonyldiimidazole (1.44 g, 8.900 mmol) at RT under N₂ atm. After stirring for 16 h, the reaction mixture was quenched by adding water (20 mL), and concentrated under reduced pressure. The aqueous layer was further diluted with water (20 mL) and extracted with ethyl acetate (25 mL×2). The combined organic layer was dried over Na₂SO₄, and concentrated under reduced pressure to give a residue. The residue was purified by column chromatography on silica gel (dichloromethane/methanol=1000 to 973) to give the title compounds (0.890 g, 63.0%) as an off white solid. The crude product was used as such for the next step without further purification. LCMS: m/z 318.9 [M+H]+.

### Intermediate 29: Cis- and trans-4-(1-(4-methoxybenzyl)-2-oxohexahydrospiro[benzo[D]imidazole-5,2′-[1,3]dioxolan]-3(2H)-yl)-2-(trifluoromethyl)benzonitrile

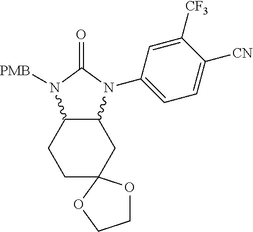

Cis- and trans-1-(4-methoxybenzyl)hexahydrospiro[benzo[d]imidazole-5,2′-[1,3]dioxolan]-2(3H)-one (0.890 g, 2.790 mmol) were reacted with 4-iodo-2-(trifluoromethyl)benzonitrile (0.830 g, 2.790 mmol) as described for the synthesis of example 1 to give a residue. The residue was purified by column chromatography on silica gel (hexanes/ethyl acetate=28) to give the title compounds (0.90 g, 66.0%) as an off white solid. The crude product was used as such for the next step without further purification.

### Intermediate 30: Cis- and trans-4-(3-(4-methoxybenzyl)-2,6-dioxooctahydro-1H-benzo[d]imidazol-1-yl)-2-(trifluoromethyl)benzonitrile

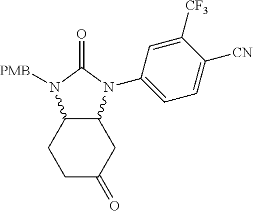

Cis- and trans-4-(1-(4-methoxybenzyl)-2-oxohexahydrospiro[benzo[d]imidazole-5,2′-[1,3]dioxolan]-3(2H)-yl)-2-(trifluoromethyl)benzonitrile (0.250 g, 0.51 mmol) were reacted with conc. HCl as described for the synthesis of example 81 to give a residue. The crude product was used as such for the next step without further purification.

### Intermediate 31: Cis- and trans-4-(6-hydroxy-3-(4-methoxybenzyl)-2-oxooctahydro-1H-benzo[d]imidazol-1-yl)-2-(trifluoromethyl)benzonitrile

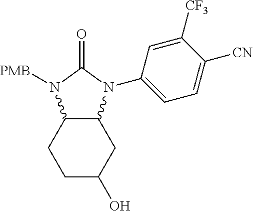

Cis- and trans-4-(3-(4-methoxybenzyl)-2,6-dioxooctahydro-1H-benzo[d]imidazol-1-yl)-2-(trifluoromethyl)benzonitrile (0.210 g 0.474) were reduced by using NaBH₄ (0.036 g, 0.948 mmol) as described for the synthesis of example 82 to give a residue. The residue was purified by column chromatography on silica gel (hexanes/ethyl acetate=73) to give the title compounds (0.20 g 95.0%) as an off white solid. LCMS: m/z 445.8 [M+H]⁺.

### Intermediate 32: Cis- and trans-4-(6-methoxy-3-(4-methoxybenzyl)-2-oxooctahydro-1H-benzo[d]imidazol-1-yl)-2-(trifluoromethyl)benzonitrile

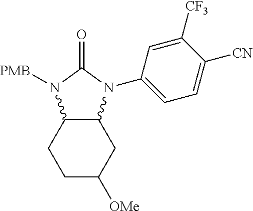

To a solution of cis- and trans-4-(6-hydroxy-3-(4-methoxybenzyl)-2-oxooctahydro-1H-benzo[d]imidazol-1-yl)-2-(trifluoromethyl)benzonitrile (0.200 g, 0.449 mmol) in THF (5 mL) was added NaH (0.036 g, 0.898 mmol) at 0° C. After stirring for 10 min at the same temperature, MeI (0.12 mL, 0.898 mmol) was added, and stirred for 1 h. The reaction mixture was poured onto ice-water (5 mL) and extracted with ethyl acetate (10 mL×2). The combined organic layer was dried over Na₂SO₄ and concentrated under reduced pressure to give the title compounds (0.200 g, crude). The crude product was used as such for the next step without further purification.

### Intermediate 33: Cis- and trans-4-(6-methoxy-2-oxooctahydro-1H-benzo[c]imidazol-1-yl)-2-(trifluoromethyl)benzonitrile

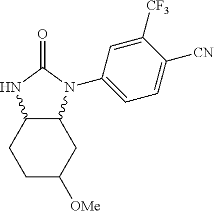 [SMILES: COC1CCC2NC(=O)N(c3ccc(C#N)c(C(F)(F)F)c3)C2C1]

A solution of cis- and trans-4-(6-methoxy-3-(4-methoxybenzyl)-2-oxooctahydro-1H-benzo[d]imidazol-1-yl)-2-(trifluoromethyl)benzonitrile (0.200 g, 0.435 mmol) in TFA (2 mL) was heated to reflux for 2 h. The reaction mixture was cooled to RT, quenched with NaHCO₃ solution (10 mL), and extracted ethyl acetate (10 mL×2). The combined organic layer was dried over Na₂SO₄ and concentrated under reduced pressure to give the title compounds (0.150 g, crude). The crude product was used as such for the next step without further purification.

### Example 85

### Trans-4-(3-(4-cyano-3-(trifluoromethyl)phenyl)-5-methoxy-2-oxooctahydro-1H-benzo[d]imidazol-1-yl)-2-fluoro-N-methylbenzamide (±)

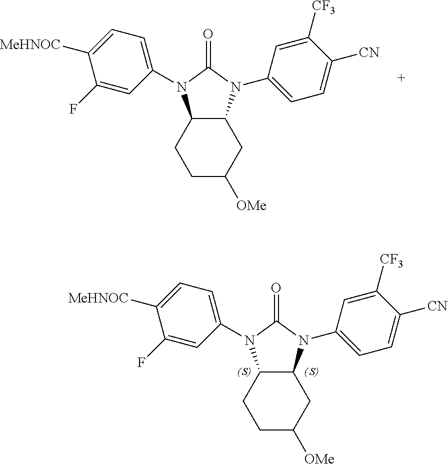

Cis- and trans-4-(6-methoxy-2-oxooctahydro-1H-benzo[d]imidazol-1-yl)-2-(trifluoromethyl)benzonitrile (0.15 g, 0.442 mmol) were reacted with 2-fluoro-4-iodo-N-methylbenzamide (0.13 g, 0.442 mmol) as described for the synthesis of example 1 to give a residue. The residue was purified by column chromatography on silica gel (hexanes/ethyl acetate=8515) to give the title compound (0.030 g, 13%) as a white solid (major product). HPLC=94.13%; LCMS: m/z 491.1 [M+H]⁺; ¹H NMR (400 MHz, CDCl₃) δ 8.13 (t, 1H), 7.83 (d, 1H), 7.70 (s, 1H), 7.49 (d, 1H), 7.12 (d, 1H), 7.04 (d, 1H), 7.67 (m, 1H), 3.74 (t, 2H), 3.52 (m, 1H), 3.40 (s, 3H), 3.0 (d, 3H), 2.71 (d, 1H), 2.39 (t, 2H) 1.50 (m, 3H).

### Intermediate 34: Trans-3-(4-cyano-3-(trifluoromethyl)phenyl)-1-(4-methoxybenzyl)-2-oxooctahydro-1H-benzo[c]imidazol-5-yl methanesulfonate (±)

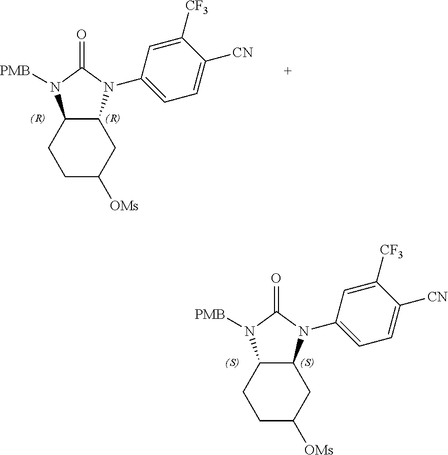

To a solution of trans-4-(6-hydroxy-3-(4-methoxybenzyl)-2-oxooctahydro-1H-benzo[d]imidazol-1-yl)-2-(trifluoromethyl)benzonitrile [racemic (±)] (0.700 g, 1.500 mmol) in DCM (10 mL) was added NEt₃ (0.54 mL, 3.900 mmol) followed by methanesulfonyl chloride (0.25 mL, 3.150 mmol) at 0° C. The reaction mixture was allowed to warm to RT and stirred for 2 h. The reaction mixture was poured onto NaHCO₃ solution (10 mL) and extracted with DCM (10 mL×2). The combined organic layer was dried over Na₂SO₄ and concentrated under reduced pressure to give the title compound (0.800 g, crude) as an off white solid. LCMS: m/z 524.1 [M+H]⁺.

### Intermediates 35 and 36: Trans-4-(3-(4-methoxybenzyl)-2-oxo-2,3,3a,4,5,7a-hexahydro-1H-benzo[d]imidazol-1-yl)-2-(trifluoromethyl)benzonitrile (±) and trans-4-(3-(4-methoxybenzyl)-2-oxo-2,3,3a,4,7,7a-hexahydro-1H-benzo[c]imidazol-1-yl)-2-(trifluoromethyl)benzonitrile (±)

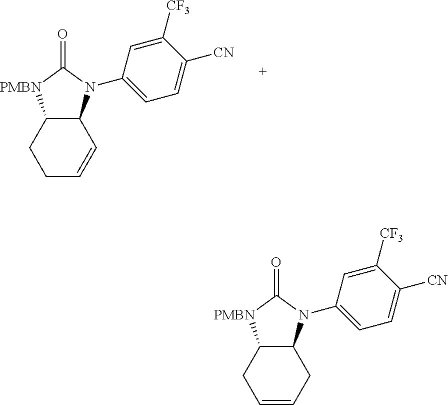

Trans-3-(4-cyano-3-(trifluoromethyl)phenyl)-1-(4-methoxybenzyl)-2-oxooctahydro-1H-benzo[d]imidazol-5-yl methanesulfonate (0.800 g, 1.500 mmol) was reacted with DBU (0.8 mL) as described for the syntheses of examples 83 and 84 to give a residue. The residue was purified by column chromatography on silica gel (hexanes/ethyl acetate=46) to give a mixture of title the compounds (0.300 g, 46%) as an off white solid. LCMS: m/z 428.3 [M+H]⁺.

### Intermediates 37 and 38: trans-4-(2-oxo-2,3,3a,4,5,7a-hexahydro-1H-benzo[d]imidazol-1-yl)-2-(trifluoromethyl)benzonitrile (±) and trans-4-(2-oxo-2,3,3a,4,7,7a-hexahydro-1H-benzo[d]imidazol-1-yl)-2-(trifluoromethyl)benzonitrile (±)

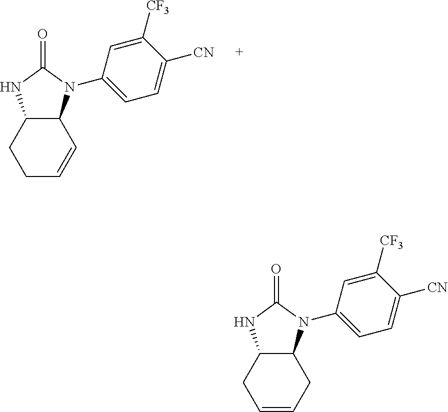 [SMILES: N#Cc1ccc(N2C(=O)N[C@H]3CCC=C[C@@H]32)cc1C(F)(F)F ; N#Cc1ccc(N2C(=O)N[C@H]3CC=CC[C@@H]32)cc1C(F)(F)F]

A mixture of intermediates 35 and 36 (0.300 g, 0.700 mmol) was dissolved in TFA (5 mL) and heated to reflux for 2 h. The reaction mixture was cooled to RT, quenched with NaHCO₃ solution (10 mL), and extracted with ethyl acetate (10 mL×2). The combined organic layer was dried over Na₂SO₄ and concentrated under reduced pressure to give a residue. The residue was purified by column chromatography on silica gel (hexanes/ethyl acetate=64) to give a mixture of the title compounds (0.200 g, 93.0%). LCMS: m/z 308.1 [M+H]+.

### Examples 86 and 83

### trans-4-(3-(4-cyano-3-(trifluoromethyl)phenyl)-2-oxo-2,3,3a,6,7,7a-hexahydro-1H-benzo[c]imidazol-1-yl)-2-fluoro-N-methylbenzamide (±) and trans-4-(3-(4-cyano-3-(trifluoromethyl)phenyl)-2-oxo-2,3,3a,4,7,7a-hexahydro-1H-benzo[d]imidazol-1-yl)-2-fluoro-N-methylbenzamide (±)

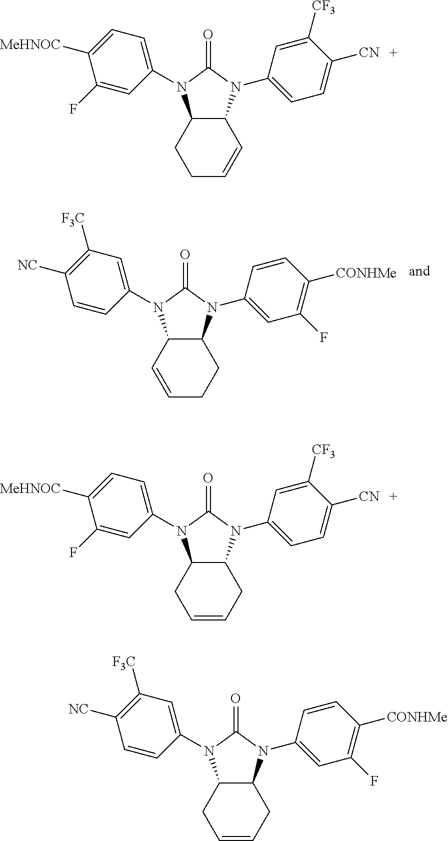

A solution of trans-4-(2-oxo-2,3,3a,4,5,7a-hexahydro-1H-benzo[d]imidazol-1-yl)-2-(trifluoromethyl)benzonitrile [racemic (±)] and trans-4-(2-oxo-2,3,3a,4,7,7a-hexahydro-1H-benzo[d]imidazol-1-yl)-2-trifluoromethyl)benzonitrile [racemic (±)] (0.250 g, 0.814 mmol) were reacted with 2-fluoro-4-iodo-N-methylbenzamide (0.241 g, 0.814 mmol) as described for the synthesis of example 1 to give a residue. The residue was purified by column chromatography on silica gel (hexanes/ethyl acetate=37) to give a mixture of the title compounds (0.180 g, 48.6%) as a white solid. The above mixture has been separated by preparative HPLC to give peak 1 as trans-4-((3aS,7aS)-3-(4-cyano-3-(trifluoromethyl)phenyl)-2-oxo-2,3,3a,6,7,7a-hexahydro-1H-benzo[d]imidazol-1-yl)-2-fluoro-N-methylbenzamide (example 86) (0.005 g) as white solid. HPLC=94.93%; LCMS: m/z 459.2[M+H]⁺; ¹H NMR (400 MHz, CDCl₃) δ 8.18 (t, 1H), 7.87 (d, 1H), 7.80 (s, 1H), 7.60 (d, 1H), 7.22 (dd, 1H), 7.09 (dd, 1H), 6.73 (m, 1H), 6.08 (d, 1H), 5.88 (d, 1H), 4.75 (d, 1H), 3.97 (ddd, 1H), 3.05 (d, 3H), 2.50 (m, 3H), 1.85 (m, 1H) and peak 2 as example 83.

COLUMN: AG/AD/PP/C18-026

FLOW: 20 ML/MIN; A: 0.01% TFA IN WATER; B: ACN:55:45::A:B.

### Examples 83a and 83b

### trans-4-3-(4-cyano-3-(trifluoromethyl)phenyl)-2-oxo-2,3,3a,4,7,7a-hexahydro-1H-benzo[c]imidazol-1-yl)-2-fluoro-N-methylbenzamide (+) and trans-4-3-(4-cyano-3-(trifluoromethyl)phenyl)-2-oxo-2,3,3a,4,7,7a-hexahydro-1H-benzo[d]imidazol-1-yl)-2-fluoro-N-methyl benzamide (−)

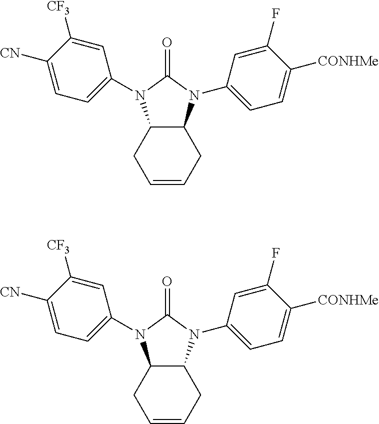

The enantiomeric mixture of example 78 was separated by preparative Chiral HPLC to give 83a trans-4-3-(4-cyano-3-(trifluoromethyl)phenyl)-2-oxo-2,3,3a,4,7,7a-hexahydro-1H-benzo[d]imidazol-1-yl)-2-fluoro-N-methylbenzamide (−) [0.040 g, retention time: 7.372 min., [α]_{D}²⁵=9 (c=0.110, MeOH), HPLC: 99.76%] and 83b trans-4-3-(4-cyano-3-(trifluoromethyl)phenyl)-2-oxo-2,3,3a,4,7,7a-hexahydro-1H-benzo[d]imidazol-1-yl)-2-fluoro-N-methylbenzamide (+) [0.040 g, retention time: 13.664 min., [α]_{D}²⁵=+5 (c=0.098, MeOH), HPLC: 98.99%] as white solids.

Column: LUXAMYLOSE-2 AXIA PACKED (21.2×250×5 u); Mobile phase: n-hexane:ethanol:: 60:40 (isocratic); Flow: 20 mL/min.

### Intermediate 39

### Cis- and trans-4-(3-methyl-2-oxohexahydrospiro[benzo[d]imidazole-5,2′-[1,3]dioxolan]-1(6H)-yl)-2-(trifluoromethyl)benzonitrile

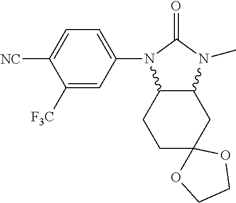 [SMILES: CN1C(=O)N(c2ccc(C#N)c(C(F)(F)F)c2)C2CCC3(CC21)OCCO3]

To a solution of cis- and trans-4-(2-oxohexahydrospiro[benzo[d]imidazole-5,2′-[1,3]dioxolan]-1(6H)-yl)-2-(trifluoromethyl)benzonitrile (3.30 g, 8.990 mmol) in THF (30 mL) was added NaH (0.72 g, 18.000 mmol) at 0° C. After stirring for 10 min, MeI (1.1 mL, 18.000 mmol) was added at 0° C., and the reaction mixture was stirred for 1 h. The reaction mixture was poured onto ice-water (25 mL) and extracted with ethyl acetate (25 mL×2). The combined organic layer was dried over Na₂SO₄ and concentrated under reduced pressure to give the title compounds (3.20 g, crude) LCMS: m/z 382.1 [M+H]+.

### Example 87

### Cis- and trans-4-(3-methyl-2,5-dioxooctahydro-1H-benzo[d]imidazol-1-yl)-2-(trifluoromethyl)benzonitrile

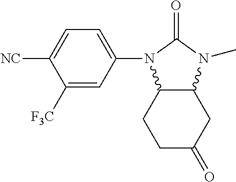 [SMILES: CN1C(=O)N(c2ccc(C#N)c(C(F)(F)F)c2)C2CCC(=O)CC21]

Cis- and trans-4-(3-methyl-2-oxohexahydrospiro[benzo[d]imidazole-5,2′-[1,3]dioxolan]-1(6H)-yl)-2-(trifluoromethyl)benzonitrile (3.20 g, 8.390 mmol) were treated with 2N HCl (10 mL) as described for the synthesis of example 81 to give the compounds (1.80 g, crude). The crude product was used as such for the next step without further purification. LCMS: m/z 338.4 [M+H]⁺.

### Intermediate 40: Cis- and trans-1-(4-cyano-3-(trifluoromethyl)phenyl)-3-methyl-2-oxo-2,3,3a,6,7,7a-hexahydro-1H-benzo[d]imidazol-5-yl trifluoromethanesulfonate

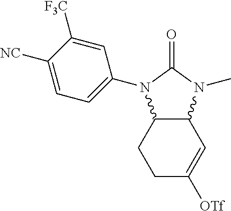 [SMILES: CN1C(=O)N(c2ccc(C#N)c(C(F)(F)F)c2)C2CCC(OS(=O)(=O)C(F)(F)F)=CC21]

To a solution of cis- and trans-4-(3-methyl-2,5-dioxooctahydro-1H-benzo[d]imidazol-1-yl)-2-(trifluoromethyl)benzonitrile (0.450 g, 1.330 mmol) in THF (10 mL) was added LDA (1.65 mL, 2M) at 0° C. and stirred for 1 h. A solution of N-phenyltrifluoromethanesulfonimide (1.10 g, 3.300 mmol) in THF (5 mL) was added dropwise at 0° C. and the reaction mixture was stirred for two days at RT. The reaction mixture was quenched with ammonium chloride solution (1 mL) and extracted with ethyl acetate (25 mL×2). The combined organic layer was dried over Na₂SO₄ and concentrated under reduced pressure to give a residue. The residue was purified by column chromatography on silica gel (hexanes/ethyl acetate=82) to give trans-1-(4-cyano-3-(trifluoromethyl)phenyl)-3-methyl-2-oxo-2,3,3a,6,7,7a-hexahydro-1H-benzo[d]imidazol-5-yl trifluoromethanesulfonate (0.045 g, 7.0%) as a light yellow solid. Further elution on silica gel (hexanes/ethyl acetate=73) to give cis-1-(4-cyano-3-(trifluoromethyl)phenyl)-3-methyl-2-oxo-2,3,3a,6,7,7a-hexahydro-1H-benzo[d]imidazol-5-yl trifluoromethanesulfonate (0.045 g, 7.0%) as a light yellow solid. LCMS: m/z 504 [M+Cl]⁻.

### Example 88

### Trans-4-(1-(4-cyano-3-(trifluoromethyl)phenyl)-3-methyl-2-oxo-2,3,3a,6,7,7a-hexahydro-1H-benzo[d]imidazol-5-yl)-2-fluoro-N-methylbenzamide (±)

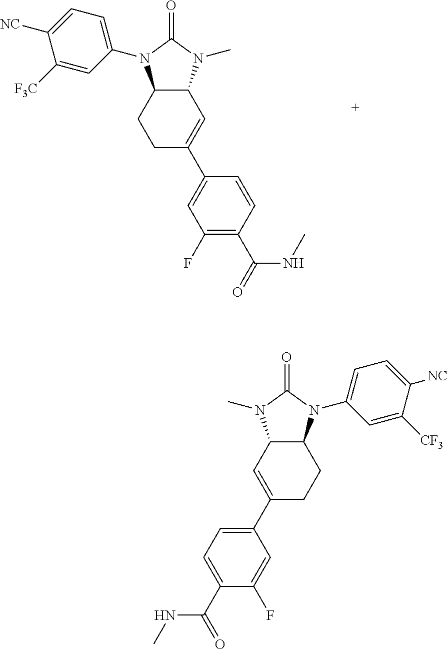

A suspension of trans-1-(4-cyano-3-(trifluoromethyl)phenyl)-3-methyl-2-oxo-2,3,3a,6,7,7a-hexahydro-1H-benzo[d]imidazol-5-yl trifluoromethanesulfonate [racemic (±)] (0.045 g, 0.01 mmol), Na₂CO₃ (0.030 g, 0.287 mmol), (3-fluoro-4-(methylcarbamoyl)phenyl)boronic acid (0.022 g, 0.114 mmol) in THF (4 mL) and H₂O (1 mL) was degassed for 30 min in a microwave vial. Tetrakis(triphenylphosphine)palladium (0) (0.011 g, 0.01 mmol) was added and the vial was sealed with an aluminum cap. The sealed vial was kept in a preheated oil bath at 100° C. for 2 h. The reaction mixture was cooled to RT, poured onto water (5 mL), and extracted with ethyl acetate (10 mL×2). The combined organic layer was dried over Na₂SO₄ and concentrated under reduced pressure to give a residue. The residue was purified by preparative TLC (hexanes/ethyl acetate=11) to give the title compound (0.010 g, 22%) as a white solid. HPLC=99.03%; LCMS: m/z 473.1[M+H]⁺; ¹H NMR (400 MHz, CDCl₃) δ 8.13 (t, 1H), 7.82 (d, 1H), 7.78 (s, 1H), 7.64 (d, 1H) 7.32 (d, 1H), 7.15 (d, 1H), 6.75 (s, 1H), 6.29 (s, 1H), 3.82-3.80 (m, 1H), 3.40-3.36 (m, 1H), 3.05 (d, 3H), 2.96 (s, 3H), 2.95 (m, 2H), 2.70-2.60 (m, 1H), 2.40-2.30 (m, 1H).

### Example 89

### Cis-4-(1-(4-cyano-3-(trifluoromethyl)phenyl)-3-methyl-2-oxo-2,3,3a,6,7,7a-hexahydro-1H-benzo[d]imidazol-5-yl)-2-fluoro-N-methylbenzamide (±)

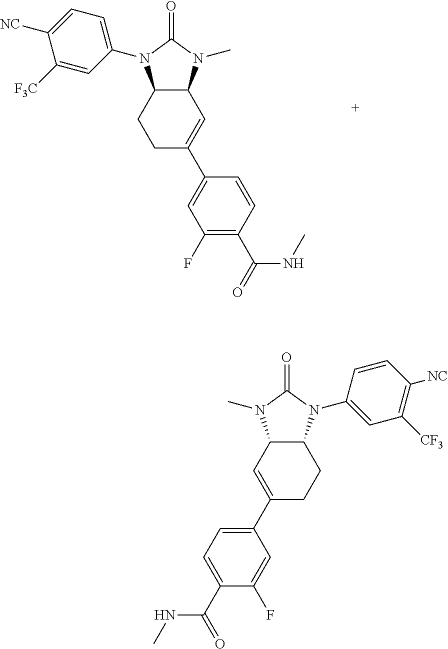

To a solution of cis-1-(4-cyano-3-(trifluoromethyl)phenyl)-3-methyl-2-oxo-2,3,3a,6,7,7a-hexahydro-1H-benzo[d]imidazol-5-yl trifluoromethanesulfonate [racemic (±)] (0.045 g, 0.0958 mmol) was reacted with 3-fluoro-4-(methylcarbamoyl)phenyl)boronic acid (0.022 g, 0.114 mmol) as described for the synthesis of example 88 to give a residue. The residue was purified by preparative TLC (ethyl acetate/hexanes=11) to give the title compound (0.015 g, 33%) as a white solid. HPLC=94.1%; LCMS: m/z 473.1 [M+H]⁺; ¹H NMR (400 MHz, CDCl₃) δ 8.13 (m, 2H), 7.80 (q, 2H), 7.32 (d, 1H), 7.14 (d, 1H), 6.75 (s, 1H), 6.26 (d, 1H), 4.75 (m, 1H), 4.24 (m, 1H), 3.05 (d, 3H), 2.98 (s, 3H), 2.52 (m, 2H), 2.21 (m, 1H), 1.91 (m, 1H).

### Intermediate 41: Cis- and trans-1-(4-methoxybenzyl)-3-methylhexahydrospiro[benzo[d]imidazole-5,2′-[1,3]dioxolan]-2(3H)-one

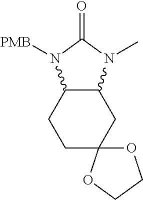

Cis- and trans-1-(4-methoxybenzyl)hexahydrospiro[benzo[d]imidazole-5,2′-[1,3]dioxolan]2(3H)-one (0.200 g, 0.620 mmol) were methylated as described for the synthesis of intermediate 32 to give the title compounds (0.200 g, 95.0%). LCMS: m/z 332.9 [M+H]⁺.

### Intermediate 42: Trans-1-(4-methoxybenzyl)-3-methyltetrahydro-1H-benzo[d]imidazole-2,5(3H,6H)-dione (±)

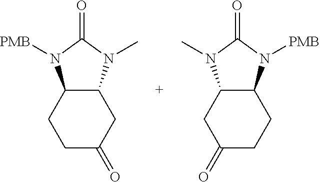

Cis- and trans-1-(4-methoxybenzyl)-3-methylhexahydrospiro[benzo[d]imidazole-5,2′-[1,3]dioxolan]-2(3H)-one (0.200 g, 0.600 mmol) were reacted with 2N HCl (2 mL) as described for the synthesis of example 81 to give a residue. The residue was purified by column chromatography on silica gel (hexanes/ethyl acetate=64) to give the title compound (0.100 g). LCMS: m/z 288.9 [M+H]+.

### Intermediate 43: Trans-5-hydroxy-1-(4-methoxybenzyl)-3-methyl-5-phenylhexahydro-1H-benzo[d]imidazol-2(3H)-one

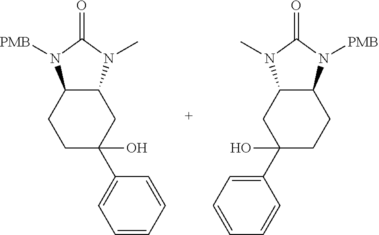

To a solution of trans-1-(4-methoxybenzyl)-3-methyltetrahydro-1H-benzo[d]imidazole-2,5(3H,6H)-dione (0.100 g, 0.365 mmol) in THF (5 mL) was added PhMgBr (0.24 mL 3M) at 10° C. The reaction mixture was allowed to warm to RT and stirred for 2 h. The reaction mixture was quenched by adding ammonium chloride solution (1 mL) and extracted with ethyl acetate (10 mL×2). The combined organic layer was dried over Na₂SO₄ and concentrated under reduced pressure to give the title compound (0.100 g). The crude product was used as such for the next step without further purification.

### Intermediate 44: Trans-3-methyl-5-phenyl-3,3a,7,7a-tetrahydro-1H-benzo[d]imidazol-2(6H)-one (±)

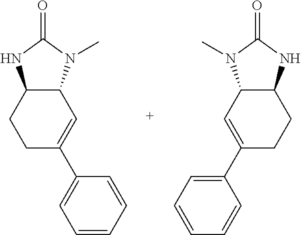 [SMILES: CN1C(=O)N[C@@H]2CCC(c3ccccc3)=C[C@H]21 ; CN1C(=O)N[C@H]2CCC(c3ccccc3)=C[C@@H]21]

Trans-5-hydroxy-1-(4-methoxybenzyl)-3-methyl-5-phenylhexahydro-1H-benzo[d]imidazol-2(3H)-one (0.100 g, 0.270 mmol) was dissolved in TFA (2 mL) and the reaction mixture was heated to reflux for 2 h. The reaction mixture was quenched with NaHCO₃ solution (10 mL) and extracted with ethyl acetate (10 mL×2). The combined organic layer was dried over Na₂SO₄ and concentrated under reduced pressure to give a residue. The residue was purified by column chromatography on silica gel (hexanes/ethyl acetate=82) to give the title compound (0.030 g, 50%). LCMS: m/z 229.0 [M+H]⁺.

### Intermediate 45: Trans-1-methyl-6-phenylhexahydro-1H-benzo[d]imidazol-2(3H)-one

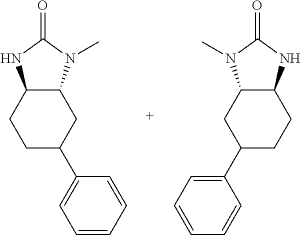 [SMILES: CN1C(=O)N[C@@H]2CCC(c3ccccc3)C[C@H]21 ; CN1C(=O)N[C@H]2CCC(c3ccccc3)C[C@@H]21]

To a solution of trans-3-methyl-5-phenyl-3,3a,7,7a-tetrahydro-1H-benzo[d]imidazol-2(6H)-one (0.030 g, 0.130 mmol) in MeOH:EtOAc (1:1, 10 mL) was added 10% Pd/C (10 mg). The resulting suspension was stirred under hydrogen atmosphere for 24 h. The suspension was filtered through a pad of celite and the filtrate was concentrated to give the title compound (0.030 g). LCMS: m/z 231.3 [M+H]⁺.

### Example 90

### Trans-4-(3-methyl-2-oxo-5-phenyloctahydro-1H-benzo[d]imidazol-1-yl)-2-(trifluoromethyl)benzonitrile

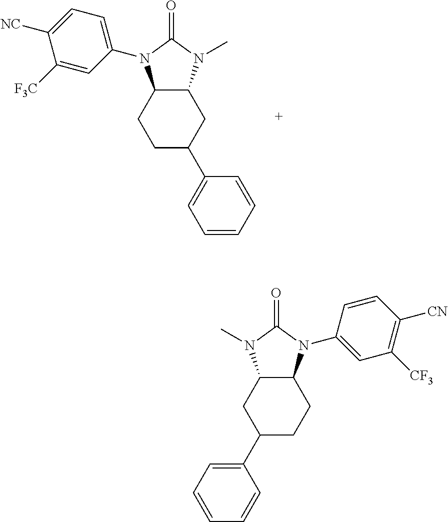 [SMILES: CN1C(=O)N(c2ccc(C#N)c(C(F)(F)F)c2)[C@@H]2CCC(c3ccccc3)C[C@H]21 ; CN1C(=O)N(c2ccc(C#N)c(C(F)(F)F)c2)[C@H]2CCC(c3ccccc3)C[C@@H]21]

Trans-1-methyl-6-phenylhexahydro-1H-benzo[d]imidazol-2(3H)-one (0.030 g, 0.130 mmol) was reacted with 4-iodo-2-(trifluoromethyl)benzonitrile (0.038 g, 0.13 mmol) as described for the synthesis of example 1 to give a residue. The residue was purified by preparative TLC (hexanes/ethyl acetate=11) to give the title compound (0.010 g, 20.0%) as an off white solid. HPLC=99.67%; LCMS: m/z 399.9[M+H]⁺; ¹H NMR (400 MHz, CDCl₃) δ 7.80 (d, 1H), 7.76 (s, 1H), 7.57 (d, 1H), 7.36 (t, 2H), 7.27 (m, 3H), 3.61 (ddd, 1H), 3.18 (ddd, 1H), 2.88 (m, 1H), 2.85 (s, 3H), 2.38 (ddd, 2H), 2.20 (d, 1H), 1.80 (m, 3H).

### Example 91

### Trans-4-3-methyl-2-oxo-5-phenyl-2,3,3a,6,7,7a-hexahydro-1H-benzo[d]imidazol-1-yl)-2-(trifluoromethyl)benzonitrile (±)

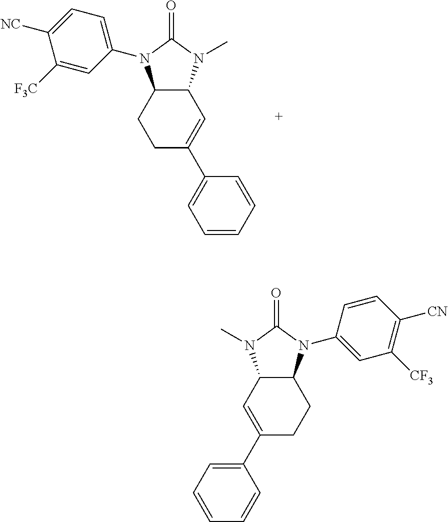 [SMILES: CN1C(=O)N(c2ccc(C#N)c(C(F)(F)F)c2)[C@@H]2CCC(c3ccccc3)=C[C@H]21 ; CN1C(=O)N(c2ccc(C#N)c(C(F)(F)F)c2)[C@H]2CCC(c3ccccc3)=C[C@@H]21]

Trans-3-methyl-5-phenyl-3,3a,7,7a-tetrahydro-1H-benzo[d]imidazol-2(6H)-one [racemic (±)] (0.040 g, 0.175 mmol) was reacted with 4-iodo-2-(trifluoromethyl)benzonitrile (0.050 g, 0.175 mmol) as described for the synthesis of example 1 to give a residue. The residue was purified by column chromatography on silica gel (hexanes/ethyl acetate=64) to give the title compound (0.030 g, 43.0%) as a yellow solid. HPLC=97.80%; LCMS: m/z 397.8 [M+H]⁺; ¹H NMR (400 MHz, CDCl₃) δ 7.82 (s, 2H), 7.65 (d, 1H), 7.38 (m, 5H), 6.14 (s, 1H), 3.81 (ddd, 1H), 3.38 (ddd, 1H), 2.99 (m, 2H), 2.95 (s, 3H), 2.66 (ddd, 1H), 2.31 (ddd, 1H).

### Biological Activity

Dihydrotestosterone (DHT) Mediated XTT Cell Proliferation Assay for Screening AR Antagonists Using Vcap cells

The Vcap cells (4×106 cells) were seeded in a T-75 flask containing DMEM with 15% FBS. After 72 hours, the media was removed; the cells were washed once and replaced with phenol red free DMEM containing 8% charcoal stripped serum. Then, the cells were starved in phenol red free DMEM with 8% charcoal stripped serum for 5 days. Following the starvation, the cells were harvested and seeded in 96 well plates (10×103 cells per well) in phenol red free DMEM with 8% charcoal stripped serum (Day 0). The cells were allowed to settle and on Day 4 they were treated with different concentrations of the compound (30 μM, 10 μM, 1 μM, 500 nM, 100 nM, 10 nM, 1 nM, 0.1 nM) in the presence of 0.2 nM DHT. DHT alone and a DMSO controls were also included. All treatments were done in triplicate. The drugs were replenished on Day 7 and the cells were allowed to grow for another 72 hours. On Day 10, the experiment was terminated by adding the XTT reagent (2,3-bis[2-methoxy-4-notro-5-sulfophenyl]-2H-tetrazolium-5-caboxyanilide inner salt) dissolved in Phenol red free DMEM without serum. The cells with the XTT reagents were incubated inside the CO₂ incubator for 3-4 hours for color development; after which the readings were taken at 465 nM using a spectramax Gemini plate reader.

Data fitting: The IC₅₀ curves for the 8 compound concentrations were calculated with GraphPad Prism software using the sigmoidal dose-response function. (Graphpad Software, San Diego, Calif., USA).

PBS: 137 nM NaCl, 2.7 mM KCl, and 10 mM PO₄

The results of the XTT assay for certain Examples are given Table 1.

TABLE 1

| Example No. | XTT (nM) |
|---|---|
| 1 | 245 |
| 1a | >10000 |
| 1b | 90.55 |
| 2 | 239.6 |
| 2a | 231.3 |
| 2b | >10000 |
| 3 | 1519 |
| 4 | 1040 |
| 5 | 1022 |
| 5a | 1760 |
| 5b | 1053 |
| 6 | >10000 |
| 7 | Not |
| | determined |
| 8 | >10000 |
| 9 | >10000 |
| 10 | 365.3 |
| 10a | 2546 |
| 10b | 279.8 |
| 11 | 1241 |
| 12 | 532.7 |
| 13 | 1049 |
| 14 | 715.3 |
| 15 | 822.7 |
| 16 | 2448 |
| 17 | >10000 |
| 18 | 373.6 |
| 19 | 232.9 |
| 20 | 321.6 |
| 21 | 254.8 |
| 22 | 196 (Rev) |
| 23 | >10000 |
| 24 | 1519 |
| 25 | 42.37 |
| 26 | 75.53 |
| 27 | 195.9 |
| 28 | 148 |
| 29 | 176.5 |
| 30 | 76.35 |
| 31 | 37.25 |
| 32 | 377 |
| 33 | 43.57 |
| 34 | 317.3 |
| 35 | 236.2 |
| 36 | 203 |
| 37 | 6085 |
| 38 | Not |
| | determined |
| 39 | 399.3 |
| 40 | >10000 |
| 41 | 134.4 |
| 42 | 167.6 |
| 43 | 339 |
| 44 | 96.39 |
| 45 | 1016 |
| 46 | 659.9 |
| 47 | 61.62 |
| 48 | >10000 |
| 49 | 300 |
| 50 | >10000 |
| 51 | 1082 |
| 52 | >10000 |
| 53 | 165 (only 65% |
| | @ high conc) |
| 54 | 244.8 |
| 55 | 174.1 |
| 56 | 1818 |
| 57 | 265.3 |
| 58 | 546.7 |
| 59 | 1079 |
| 60 | 397 |
| 61 | 348.1 |
| 62 | 2158 |
| 63 | 2290 |
| 64 | Not |
| | determined |
| 65 | Not |
| | determined |
| 66 | 260.6 |
| 67 | 1094 |
| 68 | Not |
| | determined |
| 69 | >10000 |
| 70 | 732.8 |
| 71 | 655.5 |
| 71a | >10000 |
| 71b | 379 |
| 72 | >10000 |
| 73 | 2548 |
| 74 | Not |
| | determined |
| 75 | Not |
| | determined |
| 76 | 2537 |
| 76a | >10000 |
| 76b | 149.6 |
| 77 | Not |
| | determined |
| 78 | Not |
| | Determined |
| 79 | 4331 |
| 80 | 254.5 |
| 80a | 93.68 |
| 80b | 2175 |
| 81 | 702.5 |
| 82 | 583.2 |
| 82a | >10000 |
| 82b | 311.9 |
| 83 | 147.7 |
| 83a | >10000 |
| 83b | 31.87 |
| 84 | 160.6 |
| 85 | 2617 |
| 86 | 43.05 |
| 87 | Not |
| | determined |
| 88 | 218.2 |
| 89 | 1005 |
| 90 | 372.3 |
| 91 | 323.2 |

1. A compound according to formula (I) 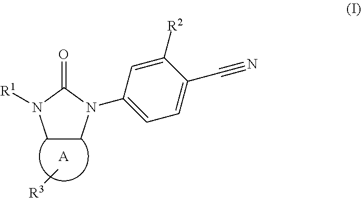
wherein:
R¹ is C₁₋₃alkyl or optionally substituted phenyl, optionally substituted benzyl, optionally substituted 2,3-dihydrobenzofuranyl, optionally substituted 5 or 6 membered heteroaryl, or optionally substituted 5 or 6 membered heteroaryl-CH₂—, wherein each ring is optionally substituted with one to three substituents each independently selected from the group consisting of: halo, cyano, hydroxy, C₁₋₃alkyl optionally substituted with one hydroxy group, C₁₋₃alkoxy, C₁₋₃haloalkyl, cyclopropyl, imidazolyl, pyrazolyl, triazolyl, tetrazolyl, morpholinyl, tetrahydrofuranyl, piperidinyl, piperazinyl, oxetanyl, C(O)R^{a}, NR^{a}R^{a}, COOR^{a}, C(O)NR^{a}R^{b}, C(O)NR^{a}OR^{c}, C(S)NR^{a}R^{b}, NR^{a}C(O)R^{a}, NHSO₂R^{a}, and SO₂NR^{a}R^{a};
R² is halo, C₁₋₃alkyl or C₁₋₃haloalkyl;
ring A is cyclohexane, cycloheptane, cyclohexene, cycloheptene, or a 6 or 7 membered saturated monocyclic heterocyclic ring having one heteroatom selected from the group consisting of O and S;
R³ is H, hydroxy, oxo, C₁₋₃alkyl, C₁₋₃alkoxy, optionally substituted phenyl, or optionally substituted 5 or 6 membered heteroaryl, wherein each ring is optionally substituted with one to three substituents each independently selected from the group consisting of: halo, cyano, hydroxy, C₁₋₃alkyl optionally substituted with one hydroxy group, C₁₋₃alkoxy, C₁₋₃haloalkyl, cyclopropyl, imidazolyl, pyrazolyl, triazolyl, tetrazolyl, morpholinyl, tetrahydrofuranyl, piperidinyl, piperazinyl, oxetanyl, C(O)R^{a}, NR^{a}R^{a}, COOR^{a}, C(O)NR^{a}R^{b}, C(O)NR^{a}OR^{c}, C(S)NR^{a}R^{b}, NR^{a}C(O)R^{a}, NHSO₂R^{a}, and SO₂NR^{a}R^{a};
R^{a} is H or C₁₋₃alkyl;
R^{b} is H, tetrahydrofuranyl, piperidinyl, piperazinyl, or oxetanyl or R^{b} is C₁₋₃alkyl optionally substituted with one or two substituents each independently selected from the group consisting of: hydroxy and C₁₋₃alkoxy;
R^{c} is C₁₋₄alkyl optionally substituted with one substituent selected from the group consisting of: hydroxy, N(CH₃)₂, N(CH₂CH₃)₂, tetrahydrofuranyl, C₁₋₄alkoxy, and C₃₋₅cycloalkyl, or R^{c} is tetrahydrofuranyl, or piperidinyl, said piperidinyl being optionally substituted with one C₁₋₃alkyl group; or a pharmaceutically acceptable salt thereof.

2. The compound according to claim 1 wherein the stereocenters denoted by a * are in a trans configuration: 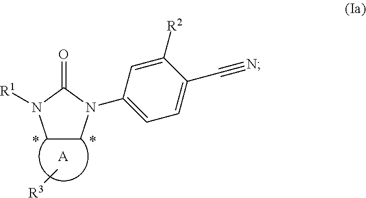
or a pharmaceutically acceptable salt thereof.

3. The compound according to claim 2 having the following formula 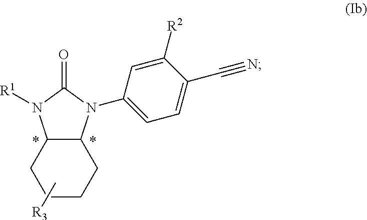
or a pharmaceutically acceptable salt thereof.

4. The compound according to claim 2 having the following formula 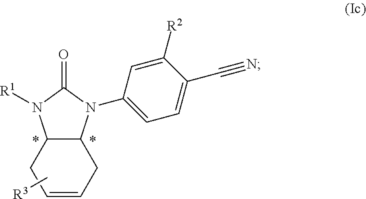
or a pharmaceutically acceptable salt thereof.

5. The compound according to claim 2 of formula (Id) or (Ie) 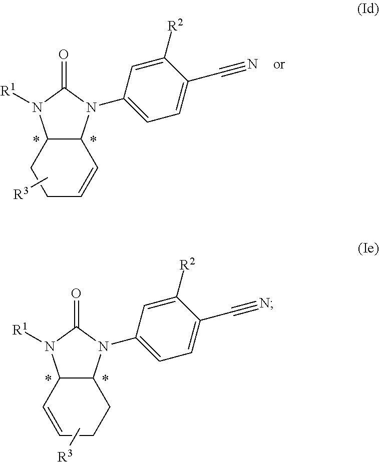
or a pharmaceutically acceptable salt thereof.

6. The compound according to claim 2 of formula (If) or (Ig) 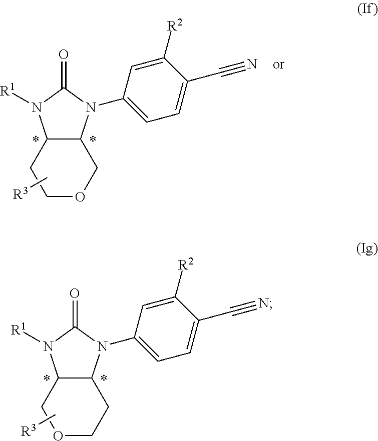
or a pharmaceutically acceptable salt thereof.

7. The compound according to claim 2 having the following formula 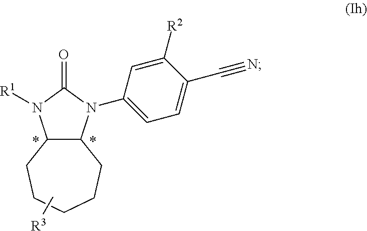
or a pharmaceutically acceptable salt thereof.

8. The compound according to claim 1 wherein R² is C₁₋₃haloalkyl; or a pharmaceutically acceptable salt thereof.

9. The compound according to claim 8 wherein R² in CF₃; or a pharmaceutically acceptable salt thereof.

10. The compound according to claim 9 wherein R³ is H; or a pharmaceutically acceptable salt thereof.

11. The compound according to claim 9 wherein R¹ is optionally substituted phenyl or optionally substituted 5 or 6 membered heteroaryl; or a pharmaceutically acceptable salt thereof.

12. The compound according to claim 11 wherein R¹ is optionally substituted phenyl, optionally substituted pyrazolyl, optionally substituted furanyl, optionally substituted thienyl, or optionally substituted pyridinyl; or a pharmaceutically acceptable salt thereof.

13. The compound according to claim 9 wherein R¹ is optionally substituted benzyl or optionally substituted 5 or 6 membered heteroaryl-CH₂—; or a pharmaceutically acceptable salt thereof.

14. The compound according to claim 13 wherein R¹ is optionally substituted benzyl or optionally substituted pyridinyl-CH₂—; or a pharmaceutically acceptable salt thereof.

15. The compound according to claim 9 wherein R¹ is 2,3-dihydrobenzofuranyl; or a pharmaceutically acceptable salt thereof.

16. The compound according to claim 1 wherein R¹ is optionally substituted phenyl, optionally substituted 2,3-dihydrobenzofuranyl, or optionally substituted 5-6 membered heteroaryl; or a pharmaceutically acceptable salt thereof.

17. The compound according to claim 16 wherein R¹ is optionally substituted phenyl, optionally substituted furan-3-yl, optionally substituted imidazol-1-yl, optionally substituted thien-3-yl, optionally substituted pyridin-2-yl, optionally substituted pyridin-3-yl, or optionally substituted pyridiny-4-yl; or a pharmaceutically acceptable salt thereof.

18. The compound according to claim 17 wherein R¹ is phenyl, furan-3-yl, imidazol-1-yl, thien-3-yl, pyridin-2-yl, pyridin-3-yl, or pyridiny-4-yl each of which is optionally substituted with one or two substituents each independently selected from the group consisting of: fluoro, chloro, cyano methyl, trifluoromethyl, cyclopropyl, imidazolyl, pyrazolyl, C(O)NHOR^{c}, NH₂, NHCH₃, COOH, C(O)CH₃, CH₂OH, COOCH₂CH₃, C(O)NR^{a}R^{b}, SO₂NH₂, NHC(O)CH₃, N(CH₃)C(O)CH₃, and NHSO₂CH₃, C(S)NHCH₃; or a pharmaceutically acceptable salt thereof.

19. The compound according to claim 18 wherein R¹ is: 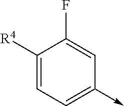
wherein the arrow indicates the point of attachment to formula (I) and R⁴ is halo, cyano, hydroxy, C₁₋₃alkyl optionally substituted with one hydroxy group, C₁₋₃alkoxy, C₁₋₃haloalkyl, cyclopropyl, imidazolyl, C(O)R^{a}, NR^{a}R^{a}, COOR^{a}, C(O)NR^{a}R^{b}, C(O)NR^{a}OR^{c}, C(S)NR^{a}R^{b}, NR^{a}C(O)R^{a}, NHSO₂R^{a}, and SO₂NR^{a}R^{a}; or a pharmaceutically acceptable salt thereof.

20. The compound according to claim 19 wherein R⁴ is C(O)NH₂, C(O)NHCH₃, or C(O)NHCH₂CH₂OH; or a pharmaceutically acceptable salt thereof.

21. The compound according to claim 1 selected from the group consisting of:
Trans-4-{3-[4-cyano-3-(trifluoromethyl)phenyl]-2-oxo-octahydro-1H-1,3-benzodiazol-1-yl}-2-fluoro-N-Methylbenzamide (±);
4-((3aS,7aS)-3-(4-cyano-3-(trifluoromethyl)phenyl)-2-oxooctahydro-1H-benzo[d]imidazol-1-yl)-2-fluorobenzamide; and
4-((3aS,7aS)-3-(4-cyano-3-(trifluoromethyl)phenyl)-2-oxooctahydro-1H-benzo[d]imidazol-1-yl)-2-fluoro-N-(2-hydroxyethyl)benzamide; or a pharmaceutically acceptable salt thereof.

22. The compound according to claim 1 which is: Trans-4-(3-(4-cyano-3-(trifluoromethyl)phenyl)-2-oxooctahydro-1H-benzo[d]im-idazol-1-yl)-N,2-dimethylbenzamide (±); or a pharmaceutically acceptable salt thereof.

23. The compound according to claim 1 which is: Trans-4-(3-(4-cyano-3-(trifluoromethyl)phenyl)-2-oxohexahydropyrano[3,4-d]imidazol-1(6H)-yl)-2-fluorobenzamide (±); or a pharmaceutically acceptable salt thereof.

24. A pharmaceutical composition comprising a compound according to claim 1 or a pharmaceutically acceptable salt thereof and a pharmaceutically acceptable carrier or excipient.

25. A method for the treatment of prostate cancer comprising administration of a therapeutically affective amount of a compound according to claim 1, or a pharmaceutically acceptable salt thereof, to a subject need of treatment thereof.
